SEQUENCE LISTING (49 sequences)

SEQ ID NO: 1 - protein, 11 aa, Artificial
  heavy chain CDR3, humanized <EGFR>ICR62
LSPGGYYVMDA

SEQ ID NO: 2 - protein, 17 aa, Artificial
  heavy chain CDR2, humanized <EGFR>ICR62
YFNPNSGYSTYAQKFQG

SEQ ID NO: 3 - protein, 5 aa, Artificial
  heavy chain CDR1, humanized <EGFR>ICR62
DYKIH

SEQ ID NO: 4 - protein, 8 aa, Artificial
  light chain CDR3, humanized <EGFR>ICR62
LQHNSFPT

SEQ ID NO: 5 - protein, 7 aa, Artificial
  light chain CDR2, humanized <EGFR>ICR62
NTNNLQT

SEQ ID NO: 6 - protein, 11 aa, Artificial
  light chain CDR1, humanized <EGFR>ICR62
RASQGINNYLN

SEQ ID NO: 7 - protein, 120 aa, Artificial
  heavy chain variable domain, humanized <EGFR>ICR62-I-HHB
QVQLVQSGAEVKKPGSSVKVSCKGSGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSS

SEQ ID NO: 8 - protein, 120 aa, Artificial [sequence omitted]

SEQ ID NO: 9 - protein, 109 aa, Artificial
  light chain variable domain, humanized <EGFR>ICR62 -I-KA
DIQMTQSPSSLSASVGDRVTITCRASQGINNYLNWYQQKPGKAPKRLIYNTNNLQTGVPSRFSGSGSGTEYTLTISSLQPEDFATYYCLQHNSFPTFGQGTKLEIKRTV

SEQ ID NO: 10 - protein, 108 aa, Artificial
  light chain variable domain, humanized <EGFR>ICR62 -I-KC
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRT

SEQ ID NO: 11 - protein, 9 aa, Artificial
  heavy chain CDR3, <IGF-1R> HUMAB-Clone 18
ELGRRYFDL

SEQ ID NO: 12 - protein, 17 aa, Artificial
  heavy chain CDR2, <IGF-1R> HUMAB-Clone 18
IIWFDGSSTYYADSVRG

SEQ ID NO: 13 - protein, 5 aa, Artificial
  heavy chain CDR1, <IGF-1R> HUMAB-Clone 18
SYGMH

SEQ ID NO: 14 - protein, 10 aa, Artificial
  light chain CDR3, <IGF-1R> HUMAB-Clone 18
QQRSKWPPWT

SEQ ID NO: 15 - protein, 7 aa, Artificial
  light chain CDR2, <IGF-1R> HUMAB-Clone 18
DASKRAT

SEQ ID NO: 16 - protein, 11 aa, Artificial
  light chain CDR1, <IGF-1R> HUMAB-Clone 18
RASQSVSSYLA

SEQ ID NO: 17 - protein, 9 aa, Artificial
  heavy chain CDR3, <IGF-1R> HUMAB-Clone 22
ELGRRYFDL

SEQ ID NO: 18 - protein, 17 aa, Artificial
  heavy chain CDR2, <IGF-1R> HUMAB-Clone 22
IIWFDGSSKYYGDSVKG

SEQ ID NO: 19 - protein, 5 aa, Artificial
  heavy chain CDR1, <IGF-1R> HUMAB-Clone 22
SYGMH

SEQ ID NO: 20 - protein, 10 aa, Artificial
  light chain CDR3, <IGF-1R> HUMAB-Clone 22
QQRSKWPPWT

SEQ ID NO: 21 - protein, 7 aa, Artificial
  light chain CDR2, <IGF-1R> HUMAB-Clone 22
DASNRAT

SEQ ID NO: 22 - protein, 11 aa, Artificial
  light chain CDR1, <IGF-1R> HUMAB-Clone 22
RASQSVSSYLA

SEQ ID NO: 23 - protein, 118 aa, Homo sapiens
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 24 - protein, 117 aa, Homo sapiens
VQLVESGGGVVQPGRSLRLSCAASGFTFSSYGMHWVRQAPGKGLEWMAIIWFDGSSKYYGDSVKGRFTISRDNSKNTLYLQMNSLRADDTAVYYCARELGRRYFDLWGRGTLVTVSS

SEQ ID NO: 25 - protein, 108 aa, Homo sapiens
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 26 - protein, 108 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK

SEQ ID NO: 27 - protein, 330 aa, Homo sapiens [sequence omitted]

SEQ ID NO: 28 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 107 aa, Homo sapiens [sequence omitted]

SEQ ID NO: 30 - protein, 450 aa, Artificial
  Heavy chain 1 of bispecific, bivalent domain exchanged <EGFR-IGF1R> antibody molecule Cross-Mab (VH/VL)
QVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 440 aa, Artificial
  Heavy chain 2 of bispecific, bivalent domain exchanged <EGFR-IGF1R> antibody molecule Cross-Mab (VH/VL)
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 32 - protein, 213 aa, Artificial
  Light chain 1 of bispecific, bivalent domain exchanged <EGFR-IGF1R> antibody molecule Cross-Mab (VH/VL)
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 225 aa, Artificial
  Light chain 2 of bispecific, bivalent domain exchanged <EGFR-IGF1R> antibody molecule Cross-Mab (VH/VL)
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 450 aa, Artificial; identical to SEQ ID NO: 30
  Heavy chain 1 of bispecific, bivalent domain exchanged <EGFR-IGF1R> antibody molecule Cross-Mab (CH/CL)

SEQ ID NO: 35 - protein, 452 aa, Artificial
  Heavy chain 2 of bispecific, bivalent domain exchanged <EGFR-IGF1R> antibody molecule Cross-Mab (CH/CL)
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 213 aa, Artificial; identical to SEQ ID NO: 32
  Light chain 1 of bispecific, bivalent domain exchanged <EGFR-IGF1R> antibody molecule Cross-Mab (CH/CL)

SEQ ID NO: 37 - protein, 213 aa, Artificial
  Light chain 2 of bispecific, bivalent domain exchanged <EGFR-IGF1R> antibody molecule Cross-Mab (CH/CL)
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 38 - protein, 695 aa, Artificial
  Heavy chain 1 of bispecific, bivalent scFab-Fc fusion <EGFR-IGF1R> antibody molecule scFab-Fc
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVQLVQSGAEVKKPGSSVKVSCKASGFTFTDYKIHWVRQAPGQCLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 695 aa, Artificial
  Heavy chain 2 of bispecific, bivalent scFab-Fc fusion <EGFR-IGF1R> antibody molecule scFab-Fc
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGCGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVELVESGGGVVQPGRSQRLSCAASGFTFSSYGMHWVRQAPGKCLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - protein, 695 aa, Artificial
  Heavy chain 1 of bispecific, bivalent scFab-Fc fusion <EGFR-IGF1R> antibody molecule N-scFabSS-Salt-bridge-s3
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSPGK

SEQ ID NO: 41 - protein, 695 aa, Artificial
  Heavy chain 2 of bispecific, bivalent scFab-Fc fusion <EGFR-IGF1R> antibody molecule N-scFabSS-Salt-bridge-s3
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGCGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVELVESGGGVVQPGRSQRLSCAASGFTFSSYGMHWVRQAPGKCLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 695 aa, Artificial
  Heavy chain 1 of bispecific, bivalent scFab-Fc fusion <EGFR-IGF1R> antibody molecule N-scFabSS-Salt bridge-w3C
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLTCLVEGFYPSDIAVEWESNGQPENNYKTTPPVLDSDGSFFLYSKLTVDKSRWQQGNVFSCSVMHEALHNHYTQESLSLSPGK

SEQ ID NO: 43 - protein, 695 aa, Artificial
  Heavy chain 2 of bispecific, bivalent scFab-Fc fusion <EGFR-IGF1R> antibody molecule N-scFabSS-Salt bridge-w3C
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGCGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVELVESGGGVVQPGRSQRLSCAASGFTFSSYGMHWVRQAPGKCLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRKKLTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 932 aa, Artificial
  Heavy chain 1 of bispecific, trivalent scFab-IgG fusion <EGFR-IGF1R> antibody molecule KiH-C-scFab-1
QVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSEIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGCGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVELVESGGGVVQPGRSQRLSCAASGFTFSSYGMHWVRQAPGKCLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 45 - protein, 450 aa, Artificial
  Heavy chain 2 of bispecific, trivalent scFab-IgG fusion <EGFR-IGF1R> antibody molecule KiH-C-scFab-1
QVQLVQSGAEVKKPGSSVKVSCKASGFTFTDYKIHWVRQAPGQGLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 213 aa, Artificial; identical to SEQ ID NO: 32
  Light chain of bispecific, trivalent scFab-IgG fusion <EGFR-IGF1R> antibody molecule KiH-C-scFab-1

SEQ ID NO: 47 - protein, 930 aa, Artificial
  Heavy chain 1 of bispecific, trivalent scFab-IgG fusion <EGFR-IGF1R> antibody molecule KiH-C-scFab-2
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGCGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGQVQLVQSGAEVKKPGSSVKVSCKASGFTFTDYKIHWVRQAPGQCLEWMGYFNPNSGYSTYAQKFQGRVTITADKSTSTAYMELSSLRSEDTAVYYCARLSPGGYYVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 48 - protein, 448 aa, Artificial
  Heavy chain 2 of bispecific, trivalent scFab-IgG fusion <EGFR-IGF1R> antibody molecule KiH-C-scFab-2
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 49 - protein, 215 aa, Artificial
  Light chain of bispecific, trivalent scFab-IgG fusion <EGFR-IGF1R> antibody molecule KiH-C-scFab-2
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2012/0149879 A1
(43) Pub. Date: Jun. 14, 2012

(54) BISPECIFIC ANTI-EGFR/ANTI-IGF-1R ANTIBODIES

(76) Inventors: Ulrich Brinkmann, Weilheim (DE); Rebecca Croasdale, Penzberg (DE); Wilma Dormeyer, Muenchen (DE); Christian Gerdes, Erlenbach/ZH (CH); Eike Hoffmann, Seefeld (DE); Christian Klein, Iffeldorf (DE); Klaus-Peter Kuenkele, Benediktbeuern (DE); Wolfgang Schaefer, Mannheim (DE); Jan Olaf Stracke, Muenchen (DE); Pablo Umana, Zuerich (CH)

(21) Appl. No.: 13/330,724

(22) Filed: Dec. 20, 2011

Related U.S. Application Data
(63) Continuation of application No. 12/565,786, filed on Sep. 24, 2009, now abandoned.

(30) Foreign Application Priority Data
Sep. 26, 2008 (EP) 08016952.7
Apr. 2, 2009 (EP) 09004908.1

(51) Int. Cl. C07K 16/46

(52) U.S. Cl. 530/387.3

(57) ABSTRACT

The present invention relates to bispecific antibodies against EGFR and against IGF-1R, methods for their production, pharmaceutical compositions containing said antibodies, and methods of treatment using the antibodies.

### PRIORITY TO RELATED APPLICATION(S)

This application is a continuation of U.S. application Ser. No. 12/565,786, filed Sep. 24, 2009, now pending; which claims the benefit of European Patent Application No. 08016952.7, filed Sep. 26, 2008, and European Patent Application No. 09004908, filed Apr. 2, 2009. The entire contents of the above-identified applications are hereby incorporated by reference.

### BACKGROUND OF THE INVENTION

The present invention relates to bispecific antibodies against EGFR and against IGF-1R, methods for their production, pharmaceutical compositions containing said antibodies, and uses thereof.

### EGFR and Anti-EGFR Antibodies

Human epidermal growth factor receptor (also known as HER-1 or Erb-B1, and referred to herein as “EGFR”) is a 170 kDa transmembrane receptor encoded by the c-erbB proto-oncogene, and exhibits intrinsic tyrosine kinase activity (Modjtahedi, H., et al., Br. J. Cancer 73 (1996) 228-235; Herbst, R. S., and Shin, D. M., Cancer 94 (2002) 1593-1611). SwissProt database entry P00533 provides the sequence of EGFR. There are also isoforms and variants of EGFR (e.g., alternative RNA transcripts, truncated versions, polymorphisms, etc.) including but not limited to those identified by Swissprot database entry numbers P00533-1, P00533-2, P00533-3, and P00533-4. EGFR is known to bind ligands including epidermal growth factor (EGF), transforming growth factor-α (TGf-α), amphiregulin, heparin-binding EGF (hb-EGF), betacellulin, and epiregulin (Herbst, R. S., and Shin, D. M., Cancer 94 (2002) 1593-1611; Mendelsohn, J., and Baselga, J., Oncogene 19 (2000) 6550-6565). EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay, G., et al., Ann. Oncology 14 (2003) 1346-1363; Tsao, A. S., and Herbst, R. S., Signal 4 (2003) 4-9; Herbst, R. S., and Shin, D. M., Cancer 94 (2002) 1593-1611; Modjtahedi, H., et al., Br. J. Cancer 73 (1996) 228-235).

Overexpression of EGFR has been reported in numerous human malignant conditions, including cancers of the bladder, brain, head and neck, pancreas, lung, breast, ovary, colon, prostate, and kidney. (Atalay, G., et al., Ann. Oncology 14 (2003) 1346-1363; Herbst, R. S., and Shin, D. M., Cancer 94 (2002) 1593-1611 Modjtahedi, H., et al., Br. J. Cancer 73 (1996) 228-235). In many of these conditions, the overexpression of EGFR correlates or is associated with poor prognosis of the patients. (Herbst R. S., and Shin, D. M., Cancer 94 (2002) 1593-1611; Modjtahedi, H., et al., Br. J. Cancer 73 (1996) 228-235). EGFR is also expressed in the cells of normal tissues, particularly the epithelial tissues of the skin, liver, and gastrointestinal tract, although at generally lower levels than in malignant cells (Herbst, R. S., and Shin, D. M., Cancer 94 (2002) 1593-1611).

Unconjugated monoclonal antibodies (mAbs) can be useful medicines for the treatment of cancer, as demonstrated by the U.S. Food and Drug Administration's approval of Trastuzumab (Herceptin™; Genentech Inc,) for the treatment of advanced breast cancer (Grillo-Lopez, A. J., et al., Semin. Oncol. 26 (1999) 66-73; Goldenberg, M. M., Clin. Ther. 21 (1999) 309-18), Rituximab (Rituxan™; IDEC Pharmaceuticals, San Diego, Calif., and Genentech Inc., San Francisco, Calif.), for the treatment of CD20 positive B-cell, low-grade or follicular Non-Hodgkin's lymphoma, Gemtuzumab (Mylotarg™, Celltech/Wyeth-Ayerst) for the treatment of relapsed acute myeloid leukemia, and Alemtuzumab (CAMPATH™, Millenium Pharmaceuticals/Schering AG) for the treatment of B cell chronic lymphocytic leukemia. The success of these products relies not only on their efficacy but also on their outstanding safety profiles (Grillo-Lopez, A. J., et al., Semin Oncol. 26 (1999) 66-73; Goldenberg, M. M., Clin. Ther. 21 (1999) 309-18). In spite of the achievements of these drugs, there is currently a large interest in obtaining higher specific antibody activity than what is typically afforded by unconjugated mAb therapy.

The results of a number of studies suggest that Fc-receptor-dependent mechanisms contribute substantially to the action of cytotoxic antibodies against tumors and indicate that an optimal antibody against tumors would bind preferentially to activation Fc receptors and minimally to the inhibitory partner FcγRIIB. (Clynes, R. A., et al., Nature Medicine 6(4) (2000) 443-446; Kalergis, A. M., and Ravetch, J. V., J. Exp. Med. 195(12) (2002) 1653-1659. For example, the results of at least one study suggest that polymorphism in the FcγRIIIa receptor, in particular, is strongly associated with the efficacy of antibody therapy. (Cartron, G., et al., Blood 99 (3) (2002) 754-758). That study showed that patients homozygous for FcγRIIIa have a better response to Rituximab than heterozygous patients. The authors concluded that the superior response was due to better in vivo binding of the antibody to FcγRIIIa, which resulted in better ADCC activity against lymphoma cells (Cartron, G., et al., Blood 99(3) (2002) 754-758).

Various strategies to target EGFR and block EGFR signaling pathways have been reported. Small-molecule tyrosine kinase inhibitors like gefitinib, erlotinib, and CI-1033 block autophosphorylation of EGFR in the intracellular tyrosine kinase region, thereby inhibiting downstream signaling events (Tsao, A. S., and Herbst, R. S., Signal 4 (2003) 4-9). Monoclonal antibodies, on the other hand, target the extracellular portion of EGFR, which results in blocking ligand binding and thereby inhibits downstream events such as cell proliferation (Tsao, A. S., and Herbst, R. S., Signal 4 (2003) 4-9).

Several murine monoclonal antibodies have been generated which achieve such a block in vitro and which have been evaluated for their ability to affect tumor growth in mouse xenograft models (Masui, H., et al., Cancer Res. 46 (1986) 5592-5598; Masui, H., et al., Cancer Res. 44 (1984) 1002-1007; Goldstein, N., et al., Clin. Cancer Res. 1 (1995) 1311-1318). For example, EMD 55900 (EMD Pharmaceuticals) is a murine anti-EGFR monoclonal antibody that was raised against human epidermoid carcinoma cell line A431 and was tested in clinical studies of patients with advanced squamous cell carcinoma of the larynx or hypopharynx (Bier, H., et al., Eur. Arch. Otohinolaryngol. 252 (1995) 433-9). In addition, the rat monoclonal antibodies ICR16, ICR62, and ICR80, which bind the extracellular domain of EGFR, have been shown to be effective at inhibiting the binding of EGF and TGF-α the receptor. (Modjtahedi, H., et al., Int. J. Cancer 75 (1998) 310-316). The murine monoclonal antibody 425 is another MAb that was raised against the human A431 carcinoma cell line and was found to bind to a polypeptide epitope on the external domain of the human epidermal growth factor receptor. (Murthy, U., et al., Arch. Biochem. Biophys. 252(2) (1987) 549-560. A potential problem with the use of murine antibodies in therapeutic treatments is that non-human monoclonal antibodies can be recognized by the human host as a foreign protein; therefore, repeated injections of such foreign antibodies can lead to the induction of immune responses leading to harmful hypersensitivity reactions. For murine-based monoclonal antibodies, this is often referred to as a Human Anti-Mouse Antibody response, or “HAMA” response, or a Human Anti-Rat Antibody, or “HARA” response. Additionally, these “foreign” antibodies can be attacked by the immune system of the host such that they are, in effect, neutralized before they reach their target site. Furthermore, non-human monoclonal antibodies (e.g., murine monoclonal antibodies) typically lack human effector functionality, i.e., they are unable to, inter alia, mediate complement dependent lysis or lyse human target cells through antibody dependent cellular toxicity or Fc-receptor mediated phagocytosis.

Chimeric antibodies comprising portions of antibodies from two or more different species (e.g., mouse and human) have been developed as an alternative to “conjugated” antibodies. For example, U.S. Pat. No. 5,891,996 (Mateo de Acosta del Rio, C. M., et al.) discusses a mouse/human chimeric antibody, R3, directed against EGFR, and U.S. Pat. No. 5,558,864 discusses generation of chimeric and humanized forms of the murine anti-EGFR MAb 425. Also, IMC-C225 (Erbitux®; ImClone) is a chimeric mouse/human anti-EGFR monoclonal antibody (based on mouse M225 monoclonal antibody, which resulted in HAMA responses in human clinical trials) that has been reported to demonstrate antitumor efficacy in various human xenograft models. (Herbst, R. S., and Shin, D. M., Cancer 94 (2002) 1593-1611). The efficacy of IMC-C225 has been attributed to several mechanisms, including inhibition of cell events regulated by EGFR signaling pathways, and possibly by increased antibody-dependent cellular toxicity (ADCC) activity (Herbst, R. S., and Shin, D. M., Cancer 94 (2002) 1593-1611). IMC-C225 was also used in clinical trials, including in combination with radiotherapy and chemotherapy (Herbst, R. S., and Shin, D. M., Cancer 94 (2002) 1593-1611). Recently, Abgenix, Inc. (Fremont, Calif.) developed ABX-EGF for cancer therapy. ABX-EGF is a fully human anti-EGFR monoclonal antibody. (Yang, X. D., et al., Crit. Rev. Oncol./Hematol. 38 (2001) 17-23).

WO 2006/082515 refers to humanized anti-EGFR monoclonal antibodies derived from the rat monoclonal antibody ICR62 and to their glycoengineered forms for cancer therapy.

### IGF-1R and Anti-IGF-1R Antibodies

Insulin-like growth factor I receptor (IGF-1R, IGF-IR, CD 221 antigen) belongs to the family of transmembrane protein tyrosine kinases (LeRoith, D., et al., Endocrin. Rev. 16 (1995) 143-163; and Adams, T. E., et al., Cell. Mol. Life. Sci. 57 (2000) 1050-1093). IGF-IR binds IGF-I with high affinity and initiates the physiological response to this ligand in vivo. IGF-IR also binds to IGF-II, however with slightly lower affinity. IGF-IR overexpression promotes the neoplastic transformation of cells and there exists evidence that IGF-IR is involved in malignant transformation of cells and is therefore a useful target for the development of therapeutic agents for the treatment of cancer (Adams, T. E., et al., Cell. Mol. Life. Sci. 57 (2000) 1050-1093).

Antibodies against IGF-IR are well-known in the state of the art and investigated for their antitumor effects in vitro and in vivo (Benini, S., et al., Clin. Cancer Res. 7 (2001) 1790-1797; Scotlandi, K., et al., Cancer Gene Ther. 9 (2002) 296-307; Scotlandi, K., et al., Int. J. Cancer 101 (2002) 11-16; Brunetti, A., et al., Biochem. Biophys. Res. Commun. 165 (1989) 212-218; Prigent, S. A., et al., J. Biol. Chem. 265 (1990) 9970-9977; Li, S. L., et al., Cancer Immunol. Immunother. 49 (2000) 243-252; Pessino, A., et al., Biochem. Biophys. Res. Commun. 162 (1989) 1236-1243; Surinya, K. H., et al., J. Biol. Chem. 277 (2002) 16718-16725; Soos, M. A., et al., J. Biol. Chem. 267 (1992) 12955-12963; Soos, M. A., et al., Proc. Natl. Acad. Sci. USA 86 (1989) 5217-5221; O'Brien, R., M., et al., EMBO J. 6 (1987) 4003-4010; Taylor, R., et al., Biochem. J. 242 (1987) 123-129; Soos, M. A., et al., Biochem. J. 235 (1986) 199-208; Li, S. L., et al., Biochem. Biophys. Res. Commun. 196 (1993) 92-98; Delafontaine, P., et al., J. Mol. Cell. Cardiol. 26 (1994) 1659-1673; Kull, F. C., Jr., et al. J. Biol. Chem. 258 (1983) 6561-6566; Morgan, D. O., and Roth, R. A., Biochemistry 25 (1986) 1364-1371; Forsayeth, J. R., et al., Proc. Natl. Acad. Sci. USA 84 (1987) 3448-3451; Schaefer, E. M., et al., J. Biol. Chem. 265 (1990) 13248-13253; Gustafson, T. A., and Rutter, W. J., J. Biol. Chem. 265 (1990) 18663-18667; Hoyne, P. A., et al., FEBS Lett. 469 (2000) 57-60; Tulloch, P. A., et al., J. Struct. Biol. 125 (1999) 11-18; Rohlik, Q. T., et al., Biochem. Biophys. Res. Comm. 149 (1987) 276-281; and Kalebic, T., et al., Cancer Res. 54 (1994) 5531-5534; Adams, T. E., et al., Cell. Mol. Life. Sci. 57 (2000) 1050-1093; Dricu, A., et al., Glycobiology 9 (1999) 571-579; Kanter-Lewensohn, L., et al., Melanoma Res. 8 (1998) 389-397; Li, S. L., et al., Cancer Immunol. Immunother. 49 (2000) 243-252). Antibodies against IGF-IR are also described in a lot of further publications, e.g., Arteaga, C. L., et al., Breast Cancer Res. Treatment 22 (1992) 101-106; and Hailey, J., et al., Mol. Cancer. Ther. 1 (2002) 1349-1353.

In particular, the monoclonal antibody against IGF-IR called αdR3 is widely used in the investigation of studying IGF-IR mediated processes and IGF-I mediated diseases such as cancer. Alpha-1R-3 was described by Kull, F. C., J. Biol. Chem. 258 (1983) 6561-6566. In the meantime, about a hundred publications have been published dealing with the investigation and therapeutic use of αdR3 in regard to its antitumor effect, alone and together with cytostatic agents such as doxorubicin and vincristine. αdR3 is a murine monoclonal antibody which is known to inhibit IGF-I binding to IGF receptor but not IGF-II binding to IGF-IR. αdR3 stimulates at high concentrations tumor cell proliferation and IGF-IR phosphorylation (Bergmann, U., et al., Cancer Res. 55 (1995) 2007-2011; Kato, H., et al., J. Biol. Chem. 268 (1993) 2655-2661). There exist other antibodies (e.g., 1H7, Li, S., L., et al., Cancer Immunol. Immunother. 49 (2000) 243-252) which inhibit IGF-II binding to IGF-IR more potently than IGF-I binding. A summary of the state of the art of antibodies and their properties and characteristics is described by Adams, T. E., et al., Cell. Mol. Life. Sci. 57 (2000) 1050-1093.

Most of the antibodies described in the state of the art are of mouse origin. Such antibodies are, as is well known in the state of the art, not useful for the therapy of human patients without further alterations like chimerization or humanization. Based on these drawbacks, human antibodies are clearly preferred as therapeutic agents in the treatment of human patients. Human antibodies are well-known in the state of the art (van Dijk, M. A., and van de Winkel, J. G., Curr. Opin. Pharmacol. 5 (2001) 368-374). Based on such technology, human antibodies against a great variety of targets can be produced. Examples of human antibodies against IGF-IR are described in WO 02/053596.

WO 2005/005635 refers to the human anti-IGF-1R antibodies <IGF-1R> HUMAB Clone 18 (DSM ACC 2587) or <IGF-1R> HUMAB Clone 22 (DSM ACC 2594) and their use in cancer therapy.

### Bispecific Antibodies

A wide variety of recombinant antibody formats have been developed in the recent past, e.g. tetravalent bispecific antibodies by fusion of, e.g., an IgG antibody format and single chain domains (see e.g. Coloma, M. J., et al., Nature Biotech 15 (1997) 159-163; WO 2001/077342; and Morrison, S. L., Nature Biotech 25 (2007) 1233-1234).

Also several other new formats wherein the antibody core structure (IgA, IgD, IgE, IgG or IgM) is no longer retained such as dia-, tria- or tetrabodies, minibodies, several single chain formats (scFv, Bis-scFv), which are capable of binding two or more antigens, have been developed (Holliger, P., et al, Nature Biotech 23 (2005) 1126-1136; Fischer, N., Leger, O., Pathobiology 74 (2007) 3-14; Shen, J., et al., Journal of Immunological Methods 318 (2007) 65-74; Wu, C., et al., Nature Biotech. 25 (2007) 1290-1297).

All such formats use linkers either to fuse the antibody core (IgA, IgD, IgE, IgG or IgM) to a further binding protein (e.g. scFv) or to fuse e.g. two Fab fragments or scFvs (Fischer, N., Leger, O., Pathobiology 74 (2007) 3-14). It has to be kept in mind that one may want to retain effector functions, such as e.g. complement-dependent cytotoxicity (CDC) or antibody dependent cellular cytotoxicity (ADCC), which are mediated through the Fc receptor binding, by maintaining a high degree of similarity to naturally occurring antibodies.

In WO 2007/024715 are reported dual variable domain immunoglobulins as engineered multivalent and multispecific binding proteins. A process for the preparation of biologically active antibody dimers is reported in U.S. Pat. No. 6,897,044. Multivalent Fᵥ antibody constructs having at least four variable domains which are linked with each over via peptide linkers are reported in U.S. Pat. No. 7,129,330. Dimeric and multimeric antigen binding structures are reported in US 2005/0079170. Tri- or tetra-valent monospecific antigen-binding protein comprising three or four Fab fragments bound to each other covalently by a connecting structure, which protein is not a natural immunoglobulin are reported in U.S. Pat. No. 6,511,663. In WO 2006/020258 tetravalent bispecific antibodies are reported that can be efficiently expressed in prokaryotic and eukaryotic cells, and are useful in therapeutic and diagnostic methods. A method of separating or preferentially synthesizing dimers which are linked via at least one interchain disulfide linkage from dimers which are not linked via at least one interchain disulfide linkage from a mixture comprising the two types of polypeptide dimers is reported in US 2005/0163782. Bispecific tetravalent receptors are reported in U.S. Pat. No. 5,959,083. Engineered antibodies with three or more functional antigen binding sites are reported in WO 2001/077342.

Multispecific and multivalent antigen-binding polypeptides are reported in WO 1997/001580. WO 1992/004053 reports homoconjugates, typically prepared from monoclonal antibodies of the IgG class which bind to the same antigenic determinant are covalently linked by synthetic cross-linking. Oligomeric monoclonal antibodies with high avidity for antigen are reported in WO 1991/06305 whereby the oligomers, typically of the IgG class, are secreted having two or more immunoglobulin monomers associated together to form tetravalent or hexavalent IgG molecules. Sheep-derived antibodies and engineered antibody constructs are reported in U.S. Pat. No. 6,350,860 which can be used to treat diseases wherein interferon gamma activity is pathogenic. In US 2005/0100543 are reported targetable constructs that are multivalent carriers of bi-specific antibodies, i.e., each molecule of a targetable construct can serve as a carrier of two or more bi-specific antibodies. Genetically engineered bispecific tetravalent antibodies are reported in WO 1995/009917. In WO 2007/109254 stabilized binding molecules that consist of or comprise a stabilized scFv are reported.

Bispecific antibodies against EGFR and IGF-1R are known from Lu, D., et al., Biochemical and Biophysical Research Communications 318 (2004) 507-513; J. Biol. Chem., 279 (2004) 2856-2865; and J. Biol. Chem. 280 (2005) 19665-72. However these bispecific anti-EGFR/anti-IGF-1R antibodies show clearly reduced tumor growth inhibition when compared with a combination of the parent monospecific antibodies (especially in tumor cells with equal (high) expression levels of both, EGFR and IGF-1R).

### SUMMARY OF THE INVENTION

We have now surprisingly found new bispecific anti-EGFR/anti-IGF-1R antibodies, which show at least similar tumor growth inhibition compared with the combination of the parent monospecific antibodies (using only a reduced amount of bispecific antibody) (especially in tumor cells with equal (high) expression levels of both, EGFR and IGF-1R).

A first aspect of the current invention is a bispecific antibody the binds to EGFR and IGF-1R comprising a first antigen-binding site that binds to EGFR and a second antigen-binding site that binds to IGF-1R, characterized in that

i) said antigen-binding sites are each a pair of an antibody heavy chain variable domain and an antibody light chain variable domain;
ii) said first antigen-binding site comprises in the heavy chain variable domain a CDR3 region of SEQ ID NO: 1, a CDR2 region of SEQ ID NO: 2, and a CDR1 region of SEQ ID NO:3, and in the light chain variable domain a CDR3 region of SEQ ID NO: 4, a CDR2 region of SEQ ID NO:5, and a CDR1 region of SEQ ID NO:6; and
iii) said second antigen-binding site comprises in the heavy chain variable domain a CDR3 region of SEQ ID NO: 11, a CDR2 region of SEQ ID NO: 12, and a CDR1 region of SEQ ID NO:13, and in the light chain variable domain a CDR3 region of SEQ ID NO: 14, a CDR2 region of SEQ ID NO:15, and a CDR1 region of SEQ ID NO:16;

or said second antigen-binding site comprises in the heavy chain variable domain a CDR3 region of SEQ ID NO: 17, a CDR2 region of SEQ ID NO: 18, and a CDR1 region of SEQ ID NO:19, and in the light chain variable domain a CDR3 region of SEQ ID NO: 20, a CDR2 region of SEQ ID NO:21, and a CDR1 region of SEQ ID NO:22.

In one embodiment of the invention the bispecific antibody is, characterized in that

i) said first antigen-binding site comprises as heavy chain variable domain SEQ ID NO: 7 or SEQ ID NO: 8, and as light chain variable domain SEQ ID NO: 9 or SEQ ID NO: 10
ii) said second antigen-binding site comprises as heavy chain variable domain SEQ ID NO: 23 or SEQ ID NO: 24, and as light chain variable domain a SEQ ID NO: 25 or SEQ ID NO: 26.

In one embodiment of the invention the bispecific antibody is, characterized in that

i) said first antigen-binding site comprises as heavy chain variable domain SEQ ID NO: 8, and as light chain variable domain SEQ ID NO: 10
ii) said second antigen-binding site comprises as heavy chain variable domain SEQ ID NO: 23, and as light chain variable domain a SEQ ID NO: 25.

Said bispecific antibodies are at least bivalent and may be trivalent, tetravalent or multivalent. Preferably the bispecific antibody according to the invention is bivalent, trivalent or tetravalent.

A further aspect of the invention is a nucleic acid molecule encoding a chain of said bispecific antibody.

Still further aspects of the invention are a pharmaceutical composition comprising said bispecific antibody, said composition for the treatment of cancer, the use of said bispecific antibody for the manufacture of a medicament for the treatment of cancer, a method of treatment of patient suffering from cancer by administering said bispecific antibody to a patient in the need of such treatment.

The bispecific antibodies according to the invention show benefits for patients in need of a EGFR and IGF-1R targeting therapy. The antibodies according to the invention have new and inventive properties causing a benefit for a patient suffering from such a disease, especially suffering from cancer.

### DESCRIPTION OF THE FIGURES

FIG. 1 Schematic structure of one tetravalent embodiment of a bispecific antibody according to the invention binding to EGFR and IGF-1R, wherein one of the Antigens A or B is EGFR, while the other is IGF-1R. The structure is based on a full length antibody binding to Antigen A, to which two (optionally disulfide-stabilized) single chain Fv's binding to Antigen B, are linked via the a peptide-linker.

FIG. 2 Schematic structure of four possible tetravalent embodiments A to D of a bispecific antibody according to the invention binding to EGFR and IGF-1R, wherein one of the Antigens A or B is EGFR, while the other is IGF-1R. The structures are based on a full length antibody binding to Antigen A, to which two (optionally disulfide stabilized) single chain Fv's binding to Antigen B, are linked via the a peptide-linker at the

A: C-terminus of the full length antibody heavy chain

B: N-terminus of the full length antibody heavy chain

C: C-terminus of the full length antibody light chain

D: C-terminus of the full length antibody light chain

FIG. 3 3a: SDS-PAGE of purified bispecific antibody XGFR1-2421

3b: HP-Size Exclusion Chromatography (SEC) analysis of purified bispecific antibody XGFR1-2421 (3 mg,/ml)

3c: HP-Size Exclusion Chromatography (SEC) analysis of purified bispecific antibody XGFR1-2421 (1 mg,/ml)

FIG. 4 4a: HP-Size Exclusion Chromatography (SEC) purification of bispecific antibody XGFR1-2320 (without disulfide stabilization) (8.7% aggregates)

4b: HP-Size Exclusion Chromatography (SEC) purification of bispecific antibody XGFR1-2321 (disulfide stabilized) (0% aggregates)

FIG. 5 Simultaneous binding of a bispecific anti-EGFR/anti-IGF-1R antibody (XGFR1-2320) to EGFR and IGF1R in a Biacore assay with immobilized XGFR1-2320

FIG. 6 Downregulation of IGFR (6a) and EGFR (6b) in A549 NSCLC tumour cell line by bispecific antibody

FIG. 7 Inhibition of IGF-1R phosphorylation (7a) and EGFR phosphorylation (7b) in H322M NSCLC tumour cell line by bispecific anti-EGFR/anti-IGF-1R antibody molecules (XGFR)

7a: Phospho-IGF-1R-ELISA after inhibition with various bispecific antibody XGFR molecules and their parent antibodies in H322M NSCLC tumour cells, antibody concentrations refer to the incubation, in case of stimulation with IGF1/EGF antibody concentrations are diluted to the half of the original concentration

7b: Phospho-EGF-R-ELISA after inhibition with various bispecific antibody XGFR molecules and their parent antibodies in H322M NSCLC tumour cells, antibody concentrations refer to the incubation, in case of stimulation with IGF1/EGF antibody concentrations are diluted to the half of the original concentration

FIG. 8 Anti-tumor growth inhibition of EGFR- and IGF-1R-expressing H322M NSCLC tumour cells by bispecific anti-EGFR/anti-IGF-1R antibody molecules (XGFR) and their parent antibodies

FIG. 9 In-vitro ADCC activity of bispecific anti-EGFR/anti-IGF-1R antibody molecules (XGFR)

FIG. 10 Schematic structure of a full length antibody without CH4 domain specifically binding to a EGFR or IGF1-R with two pairs of heavy and light chain which comprise variable and constant domains in a typical order.

FIG. 11 Schematic structure of the four possible single chain Fab fragments specifically binding e.g. to EGFR or IGF1-R

FIG. 12 Schematic structure of a tetravalent, bispecific antibodies according to the invention comprising a full length antibody specifically binding to one of the two antigens EGFR or IGF1-R and two single chain Fabs specifically binding to the other of the two antigens EGFR or IGF1-R (scFab-XGFR molecules)

FIG. 13 Bispecific antibodies according to the invention comprising a full length antibody specifically binding to IGF-1R and two identical single chain Fabs specifically binding to EGFR-ScFab-XGFR1 molecules A, B, C, and D and expression levels after purification

A: scFab (VH-CH1-linker-VL-CL) fused to C-Terminus of heavy chain

B: scFab (VH-CH1-linker-VL-CL with additional VH44-VL100 disulfide bridge fused) to C-Terminus of heavy chain

C: scFab (VH-CH1-linker-VL-CL) fused to C-Terminus of light chain

D: scFab (VH-CH1-linker-VL-CL with additional VH44-VL100 disulfide bridge fused) to C-Terminus of light chain

FIG. 14 Bispecific antibodies according to the invention comprising a full length antibody specifically binding to EGFR and two identical single chain Fabs specifically binding to IGF-1R-ScFab-XGFR2 molecules A, B, C, and D

A: scFab (VH-CH1-linker-VL-CL) fused to C-Terminus of heavy chain

B: scFab (VH-CH1-linker-VL-CL with additional VH44-VL100 disulfide bridge fused) to C-Terminus of heavy chain

C: scFab (VH-CH1-linker-VL-CL) fused to C-Terminus of light chain

D: scFab (VH-CH1-linker-VL-CL with additional VH44-VL100 disulfide bridge fused) to C-Terminus of light chain

FIG. 15 SDS-PAGE analyses of single chain Fab containing bispecific antibody derivatives scFab-XGFR1

1: scFab-XGFR14720 (Not reduced)

2: scFab-XGFR14721 (Not reduced)

3: scFab-XGFR14720 (reduced)

4: scFab-XGFR14721 (reduced)

FIG. 16 HP-SEC analyses of scFab containing bispecific antibody derivatives scFab-XGFR1

FIG. 16a: scFab-XGFR1-4720; 7.7%, Aggregates (marked within box)

FIG. 16b: scFab-XGFR1-4721; 3.5%, Aggregates (marked within box)

FIG. 17 Binding of scFab-XGFR1 and scFab-XGFR2 to EGFR and IGF1R

FIG. 17a: Biacore diagram-Binding of scFab-XGFR1_{—}2720 to EGFR, KD=2 nM

FIG. 17b: Biacore diagram-Binding of scFab-XGFR1_{—}2720 to IGF-1R, KD=2 nM

FIG. 17c: Biacore diagram-Binding of scFab-XGFR2_{—}2720 to EGFR, KD=0.5 nM

FIG. 17d: Biacore diagram-Binding of scFab-XGFR2_{—}2720 to IGF-1R, KD=11 nM

FIG. 18 Scheme-Binding of scFab-XGFR to cells analyzed by FACS competition assays with following general procedure:

add <IGF1R> Mab labeled with-Alexa647 (1 μg/mL)+unlabeled scFab-XGFR (100 μg/mL-0.001 μg/mL) in parallel

45 min incubation on ice, wash & remove unbound antibodies

fix with 1% HCHO, then FACS

FIG. 19 Binding of scFab-XGFR 2721 and parent <IGF1R> Clone18 to cells analyzed by FACS competition assays

FIG. 19a: Comparison of IC50 values of <IGF-1R> Clone18 (0.18 μg/ml) and scFab-XGFR 2721 (0.15 μg/ml)

FIG. 19b: Binding curve of <IGF-1R> Clone18 (turning point 0.11 μg/ml)−y-axis=RLU; x-axis antibody concentration (μg/ml)

FIG. 19c: Binding curve of scFab-XGFR 2721 (turning point 0.10 μg/ml)−y-axis=RLU; x-axis antibody concentration (μg/ml)

FIG. 20 Downregulation of IGF1-R on H322M-Cells after 24 h incubation with different bispecific anti-EGFR/anti-IGF-1R antibody molecules (scFab-XGFR; 100 nM)

FIG. 21 Downregulation of EGFR on H322M-Cells after 24 h incubation with different bispecific anti-EGFR/anti-IGF-1R antibody molecules (scFab-XGFR; 100 nM)

FIG. 22 Inhibition of the proliferation from H322M-cells with different bispecific anti-EGFR/anti-IGF-1R antibody molecules (scFab-XGFR; 100 nM)

### DESCRIPTION OF THE SEQUENCES

SEQ ID NO: 1 heavy chain CDR3, humanized <EGFR>ICR62
SEQ ID NO: 2 heavy chain CDR2, humanized <EGFR>ICR62
SEQ ID NO: 3 heavy chain CDR1, humanized <EGFR>ICR62
SEQ ID NO: 4 light chain CDR3, humanized <EGFR>ICR62
SEQ ID NO: 5 light chain CDR2, humanized <EGFR>ICR62
SEQ ID NO: 6 light chain CDR1, humanized <EGFR>ICR62
SEQ ID NO: 7 heavy chain variable domain, humanized <EGFR>ICR62-1-HHB
SEQ ID NO: 8 heavy chain variable domain, humanized <EGFR>ICR62-1-HHD
SEQ ID NO: 9 light chain variable domain, humanized <EGFR>ICR62-I-KA
SEQ ID NO: 10 light chain variable domain, humanized <EGFR>ICR62-I-KC
SEQ ID NO: 11 heavy chain CDR3, <IGF-1R> HUMAB-Clone 18
SEQ ID NO: 12 heavy chain CDR2, <IGF-1R> HUMAB-Clone 18
SEQ ID NO: 13 heavy chain CDR1, <IGF-1R> HUMAB-Clone 18
SEQ ID NO: 14 light chain CDR3, <IGF-1R> HUMAB-Clone 18
SEQ ID NO: 15 light chain CDR2, <IGF-1R> HUMAB-Clone 18
SEQ ID NO: 16 light chain CDR1, <IGF-1R> HUMAB-Clone 18
SEQ ID NO: 17 heavy chain CDR3, <IGF-1R> HUMAB-Clone 22
SEQ ID NO: 18 heavy chain CDR2, <IGF-1R> HUMAB-Clone 22
SEQ ID NO: 19 heavy chain CDR1, <IGF-1R> HUMAB-Clone 22
SEQ ID NO: 20 light chain CDR3, <IGF-1R> HUMAB-Clone 22
SEQ ID NO: 21 light chain CDR2, <IGF-1R> HUMAB-Clone 22
SEQ ID NO: 22 light chain CDR1, <IGF-1R> HUMAB-Clone 22
SEQ ID NO: 23 heavy chain variable domain, <IGF-1R> HUMAB-Clone 18
SEQ ID NO: 24 heavy chain variable domain, <IGF-1R> HUMAB-Clone 22
SEQ ID NO: 25 light chain variable domain, <IGF-1R> HUMAB-Clone 18
SEQ ID NO: 26 light chain variable domain, <IGF-1R> HUMAB-Clone 22
SEQ ID NO: 27 human heavy chain constant region derived from IgG1
SEQ ID NO: 28 human heavy chain constant region derived from IgG4
SEQ ID NO: 29 kappa light chain constant region
SEQ ID NO: 30 Heavy chain 1 of bispecific, bivalent domain exchanged <EGFR-IGF1R> antibody molecule: Cross-Mab (VH/VL)
SEQ ID NO: 31 Heavy chain 2 of bispecific, bivalent domain exchanged <EGFR-IGF1R> antibody molecule: Cross-Mab (VH/VL)
SEQ ID NO: 32 Light chain 1 of bispecific, bivalent domain exchanged <EGFR-IGF1R> antibody molecule: Cross-Mab (VH/VL)
SEQ ID NO: 33 Light chain 2 of bispecific, bivalent domain exchanged <EGFR-IGF1R> antibody molecule: Cross-Mab (VH/VL)
SEQ ID NO: 34 Heavy chain 1 of bispecific, bivalent domain exchanged <EGFR-IGF1R> antibody molecule: Cross-Mab (CH/CL)
SEQ ID NO: 35 Heavy chain 2 of bispecific, bivalent domain exchanged <EGFR-IGF1R> antibody molecule: Cross-Mab (CH/CL)
SEQ ID NO: 36 Light chain 1 of bispecific, bivalent domain exchanged <EGFR-IGF1R> antibody molecule: Cross-Mab (CH/CL)
SEQ ID NO: 37 Light chain 2 of bispecific, bivalent domain exchanged <EGFR-IGF1R> antibody molecule: Cross-Mab (CH/CL)
SEQ ID NO: 38 Heavy chain 1 of bispecific, bivalent scFab-Fc fusion <EGFR-IGF1R> antibody molecule: scFab-Fc
SEQ ID NO: 39 Heavy chain 2 of bispecific, bivalent scFab-Fc fusion <EGFR-IGF1R> antibody molecule: scFab-Fc
SEQ ID NO: 40 Heavy chain 1 of bispecific, bivalent scFab-Fc fusion <EGFR-IGF1R> antibody molecule: N-scFabSS-Salt-bridge-s3
SEQ ID NO: 41 Heavy chain 2 of bispecific, bivalent scFab-Fc fusion <EGFR-IGF1R> antibody molecule: N-scFabSS-Salt-bridge-s3
SEQ ID NO: 42 Heavy chain 1 of bispecific, bivalent scFab-Fc fusion <EGFR-IGF1R> antibody molecule: N-scFabSS-Salt bridge-w3C
SEQ ID NO: 43 Heavy chain 2 of bispecific, bivalent scFab-Fc fusion <EGFR-IGF1R> antibody molecule: N-scFabSS-Salt bridge-w3C
SEQ ID NO: 44 Heavy chain 1 of bispecific, trivalent scFab-IgG fusion <EGFR-IGF1R> antibody molecule: KiH-C-scFab-1
SEQ ID NO: 45 Heavy chain 2 of bispecific, trivalent scFab-IgG fusion <EGFR-IGF1R> antibody molecule: KiH-C-scFab-1
SEQ ID NO: 46 Light chain of bispecific, trivalent scFab-IgG fusion <EGFR-IGF1R> antibody molecule: KiH-C-scFab-1
SEQ ID NO: 47 Heavy chain 1 of bispecific, trivalent scFab-IgG fusion <EGFR-IGF1R> antibody molecule: KiH-C-scFab-2
SEQ ID NO: 48 Heavy chain 2 of bispecific, trivalent scFab-IgG fusion <EGFR-IGF1R> antibody molecule: KiH-C-scFab-2
SEQ ID NO: 49 Light chain of bispecific, trivalent scFab-IgG fusion <EGFR-IGF1R> antibody molecule: KiH-C-scFab-2

### DETAILED DESCRIPTION OF THE INVENTION

The present invention relates in part to a bispecific antibody the binds to EGFR and IGF-1R comprising a first antigen-binding site that binds to EGFR and a second antigen-binding site that binds to IGF-1R, characterized in that

i) said antigen-binding sites are each a pair of an antibody heavy chain variable domain and an antibody light chain variable domain;
ii) said first antigen-binding site comprises in the heavy chain variable domain a CDR3 region of SEQ ID NO: 1, a CDR2 region of SEQ ID NO: 2, and a CDR1 region of SEQ ID NO:3, and in the light chain variable domain a CDR3 region of SEQ ID NO: 4, a CDR2 region of SEQ ID NO:5, and a CDR1 region of SEQ ID NO:6; and
iii) said second antigen-binding site comprises in the heavy chain variable domain a CDR3 region of SEQ ID NO: 11, a CDR2 region of SEQ ID NO: 12, and a CDR1 region of SEQ ID NO:13, and in the light chain variable domain a CDR3 region of SEQ ID NO: 14, a CDR2 region of SEQ ID NO:15, and a CDR1 region of SEQ ID NO:16;

or said second antigen-binding site comprises in the heavy chain variable domain a CDR3 region of SEQ ID NO: 17, a CDR2 region of SEQ ID NO: 18, and a CDR1 region of SEQ ID NO:19, and in the light chain variable domain a CDR3 region of SEQ ID NO: 20, a CDR2 region of SEQ ID NO:21, and a CDR1 region of SEQ ID NO:22.

One embodiment of the invention is a bispecific antibody binding to EGFR and IGF-1R comprising a first antigen-binding site that binds to EGFR and a second antigen-binding site that binds to IGF-1R, characterized in that

i) said antigen-binding sites are each a pair of an antibody heavy chain variable domain and an antibody light chain variable domain;
ii) said first antigen-binding site comprises in the heavy chain variable domain a CDR3 region of SEQ ID NO: 1, a CDR2 region of SEQ ID NO: 2, and a CDR1 region of SEQ ID NO: 3, and in the light chain variable domain a CDR3 region of SEQ ID NO: 4, a CDR2 region of SEQ ID NO:5, and a CDR1 region of SEQ ID NO: 6; and
iii) said second antigen-binding site comprises in the heavy chain variable domain a CDR3 region of SEQ ID NO: 11, a CDR2 region of SEQ ID NO: 12, and a CDR1 region of SEQ ID NO:13, and in the light chain variable domain a CDR3 region of SEQ ID NO: 14, a CDR2 region of SEQ ID NO:15, and a CDR1 region of SEQ ID NO:16.

Another embodiment of the invention is a bispecific antibody binding to EGFR and IGF-1R comprising a first antigen-binding site that binds to EGFR and a second antigen-binding site that binds to IGF-1R, characterized in that

i) said antigen-binding sites are each a pair of an antibody heavy chain variable domain and an antibody light chain variable domain;
ii) said first antigen-binding site comprises in the heavy chain variable domain a CDR3 region of SEQ ID NO: 1, a CDR2 region of SEQ ID NO: 2, and a CDR1 region of SEQ ID NO:3, and in the light chain variable domain a CDR3 region of SEQ ID NO: 4, a CDR2 region of SEQ ID NO:5, and a CDR1 region of SEQ ID NO:6; and
iii) said second antigen-binding site comprises in the heavy chain variable domain a CDR3 region of SEQ ID NO: 17, a CDR2 region of SEQ ID NO: 18, and a CDR1 region of SEQ ID NO:19, and in the light chain variable domain a CDR3 region of SEQ ID NO: 20, a CDR2 region of SEQ ID NO:21, and a CDR1 region of SEQ ID NO:22.

Another embodiment of the invention is a bispecific antibody binding to EGFR and IGF-1R comprising a first antigen-binding site that binds to EGFR and a second antigen-binding site that binds to IGF-1R, characterized in that

i) said antigen-binding sites are each a pair of an antibody heavy chain variable domain and an antibody light chain variable domain;
ii) said first antigen-binding site comprises as heavy chain variable domain SEQ ID NO: 7 or SEQ ID NO: 8, and as light chain variable domain SEQ ID NO: 9 or SEQ ID NO: 10
iii) said second antigen-binding site comprises as heavy chain variable domain SEQ ID NO: 23 or SEQ ID NO: 24, and as light chain variable domain a SEQ ID NO: 25 or SEQ ID NO: 26.

Another embodiment of the invention is a bispecific antibody binding to EGFR and IGF-1R comprising a first antigen-binding site that binds to EGFR and a second antigen-binding site that binds to IGF-1R, characterized in that

i) said antigen-binding sites are each a pair of an antibody heavy chain variable domain and an antibody light chain variable domain;
ii) said first antigen-binding site comprises as heavy chain variable domain SEQ ID NO: 7, and as light chain variable domain SEQ ID NO: 10,
iii) said second antigen-binding site comprises as heavy chain variable domain SEQ ID NO: 23, and as light chain variable domain a SEQ ID NO: 25.

Another embodiment of the invention is a bispecific antibody binding to EGFR and IGF-1R comprising a first antigen-binding site that binds to EGFR and a second antigen-binding site that binds to IGF-1R, characterized in that

i) said antigen-binding sites are each a pair of an antibody heavy chain variable domain and an antibody light chain variable domain;
ii) said first antigen-binding site comprises as heavy chain variable domain SEQ ID NO: 8, and as light chain variable domain SEQ ID NO: 10,
iii) said second antigen-binding site comprises as heavy chain variable domain SEQ ID NO: 23, and as light chain variable domain a SEQ ID NO: 25.

Another embodiment of the invention is a bispecific antibody binding to EGFR and IGF-1R comprising a first antigen-binding site that binds to EGFR and a second antigen-binding site that binds to IGF-1R, characterized in that

i) said antigen-binding sites are each a pair of an antibody heavy chain variable domain and an antibody light chain variable domain;
ii) said first antigen-binding site comprises as heavy chain variable domain SEQ ID NO: 7, and as light chain variable domain SEQ ID NO: 10,
iii) said second antigen-binding site comprises as heavy chain variable domain SEQ ID NO: 24, and as light chain variable domain a SEQ ID NO: 26.

Another embodiment of the invention is a bispecific antibody binding to EGFR and IGF-1R comprising a first antigen-binding site that binds to EGFR and a second antigen-binding site that binds to IGF-1R, characterized in that

i) said antigen-binding sites are each a pair of an antibody heavy chain variable domain and an antibody light chain variable domain;
ii) said first antigen-binding site comprises as heavy chain variable domain SEQ ID NO: 8, and as light chain variable domain SEQ ID NO: 10,
iii) said second antigen-binding site comprises as heavy chain variable domain SEQ ID NO: 24, and as light chain variable domain a SEQ ID NO: 26.

As used herein, “antibody” refers to a binding protein that comprises antigen-binding sites. The terms “binding site” or “antigen-binding site” as used herein denotes the region(s) of an antibody molecule to which a ligand actually binds. The binding sites in an antibody according to the invention may be each formed by a pair of two variable domains, i.e. of one heavy chain variable domain and one light chain variable domain. The minimal binding site determinant in an antibody is the heavy chain CDR3 region. In one embodiment of the current invention each of the binding sites comprises an antibody heavy chain variable domain (VH) and/or an antibody light chain variable domain (VL), and preferably is formed by a pair consisting of an antibody light chain variable domain (VL) and an antibody heavy chain variable domain (VH).

Antibody specificity refers to selective recognition of the antibody for a particular epitope of an antigen. Natural antibodies, for example, are monospecific. “Bispecific antibodies” according to the invention are antibodies which have two different antigen-binding specificities. Where an antibody has more than one specificity, the recognized epitopes may be associated with a single antigen or with more than one antigen. Antibodies of the present invention are specific for two different antigens, i.e. EGFR as first antigen and IGF-1R as second antigen.

The term “monospecific” antibody as used herein denotes an antibody that has one or more binding sites each of which bind to the same epitope of the same antigen.

The term “valent” as used within the current application denotes the presence of a specified number of binding sites in an antibody molecule. As such, the terms “bivalent”, “tetravalent”, and “hexavalent” denote the presence of two binding site, four binding sites, and six binding sites, respectively, in an antibody molecule. The bispecific antibodies according to the invention are at least “bivalent” and may be “trivalent” or “multivalent” (e.g. (“tetravalent” or “hexavalent”). Preferably the bispecific antibody according to the invention is bivalent, trivalent or tetravalent. In one embodiment said bispecific antibody is bivalent. In one embodiment said bispecific antibody is trivalent. In one embodiment said bispecific antibody is tetravalent

Antibodies of the present invention have two or more binding sites and are bispecific. That is, the antibodies may be bispecific even in cases where there are more than two binding sites (i.e. that the antibody is trivalent or multivalent). Bispecific antibodies of the invention include, for example, multivalent single chain antibodies, diabodies and triabodies, as well as antibodies having the constant domain structure of full length antibodies to which further antigen-binding sites (e.g., single chain Fv, a VH domain and/or a VL domain, Fab, or (Fab)₂) are linked via one or more peptide-linkers. The antibodies can be full length from a single species, or be chimerized or humanized. For an antibody with more than two antigen binding sites, some binding sites may be identical, so long as the protein has binding sites for two different antigens. That is, whereas a first binding site is specific for a EGFR, a second binding site is specific for IGF-1R.

Like natural antibodies, an antigen binding sites of an antibody of the invention typically contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences. Also included within the scope of the invention are functional antigen binding sites comprised of fewer CDRs (i.e., where binding specificity is determined by three, four or five CDRs). For example, less than a complete set of 6 CDRs may be sufficient for binding. In some cases, a VH or a VL domain will be sufficient.

In certain embodiments, antibodies of the invention further comprise immunoglobulin constant regions of one or more immunoglobulin classes. Immunoglobulin classes include IgG, IgM, IgA, IgD, and IgE isotypes and, in the case of IgG and IgA, their subtypes. In a preferred embodiment, an antibody of the invention has a constant domain structure of an IgG type antibody, but has four antigen binding sites. This is accomplished by linking two complete antigen binding sites (e.g., a single chain Fv) specifically binding to EGFR to either to N- or C-terminus heavy or light chain of a full antibody specifically binding to IGF-1R. The four antigen-binding sites preferably comprise two binding sites for each of two different binding specificities.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties according to the invention, especially in regard to C1q binding and/or Fc receptor (FcR) binding. Such chimeric antibodies are also referred to as “class-switched antibodies.”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See, e.g., Morrison, S. L., et al., Proc. Natl. Acad. Sci. USA 81 (1984) 6851-6855; U.S. Pat. No. 5,202,238 and U.S. Pat. No. 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G. J. Mol. Biol. 227 (1992) 381-388; Marks, J. D., et al., J. Mol. Biol. 222 (1991) 581-597). The techniques of Cole, et al. and Boerner, et al. are also available for the preparation of human monoclonal antibodies (Cole, S. P. C., et al., Monoclonal Antibodies and Cancer Therapy, Alan R. Liss (1985) 77-96; and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation.)

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo. The “variable domain” (variable domain of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play a particularly important role in the binding specificity/affinity of the antibodies according to the invention and therefore provide a further object of the invention.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. CDRs on each chain are separated by such framework amino acids. Especially, CDR3 of the heavy chain is the region which contributes most to antigen binding. CDR and FR regions are determined according to the standard definition of Kabat et al., Sequences of Proteins of Immunological Interest, 5th ed., Public Health Service, National Institutes of Health, Bethesda, Md. (1991).

The bispecific antibodies according to the invention include, in addition, such antibodies having “conservative sequence modifications” (which is meant by “variants” of the bispecific antibodies). This means nucleotide and amino acid sequence modifications which do not affect or alter the above-mentioned characteristics of the antibody according to the invention. Modifications can be introduced by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), nonpolar side chains (e.g. alanine, valine, leucine, isoleucine, proline, phenylalanine, methionine), beta-branched side chains (e.g. threonine, valine, isoleucine) and aromatic side chains (e.g. tyrosine, phenylalanine, tryptophan, histidine). Thus, a predicted nonessential amino acid residue in a bispecific <EGFR-IGF1R> antibody can be preferably replaced with another amino acid residue from the same side chain family. A “variant” bispecific <EGFR-IGF1R> antibody, refers therefore herein to a molecule which differs in amino acid sequence from a “parent” bispecific <EGFR-IGF1R> antibody amino acid sequence by up to ten, preferably from about two to about five, additions, deletions and/or substitutions in one or more variable region or constant region of the parent antibody Amino acid substitutions can be performed by mutagenesis based upon molecular modeling as described by Riechmann, L., et al., Nature 332 (1988) 323-327 and Queen, C., et al., Proc. Natl. Acad. Sci. USA 86 (1989) 10029-10033.

Identity or homology with respect to the sequence is defined herein as the percentage of amino acid residues in the candidate sequence that are identical with the parent sequence, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind human EGFR and human IGF-1R.

As used herein, the term “binding” or “specifically binding” refers to the binding of the antibody to an epitope of an antigen in an in vitro assay, preferably in a cell-based ELISA with CHO cells expressing wild-type antigen. Binding means a binding affinity (K_{D}) of 10⁻⁸ M or less, preferably 10⁻¹³ M to 10⁻⁹ M. Binding of the antibody to the antigen or FcγRIII can be investigated by a BIAcore assay (Pharmacia Biosensor AB, Uppsala, Sweden). The affinity of the binding is defined by the terms ka (rate constant for the association of the antibody from the antibody/antigen complex), k_{D} (dissociation constant), and K_{D} (k_{D}/ka).

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules.

Human epidermal growth factor receptor (also known as HER-1 or Erb-B1, and referred to herein as “EGFR”) is a 170 kDa transmembrane receptor encoded by the c-erbB proto-oncogene, and exhibits intrinsic tyrosine kinase activity (Modjtahedi, H., et al., Br. J. Cancer 73 (1996) 228-235; Herbst, R. S., and Shin, D. M., Cancer 94 (2002) 1593-1611). SwissProt database entry P00533 provides the sequence of EGFR. There are also isoforms and variants of EGFR (e.g., alternative RNA transcripts, truncated versions, polymorphisms, etc.) including but not limited to those identified by Swissprot database entry numbers P00533-1, P00533-2, P00533-3, and P00533-4. EGFR is known to bind ligands including α), epidermal growth factor (EGF), transforming growth factor-α (TGf-amphiregulin, heparin-binding EGF (hb-EGF), betacellulin, and epiregulin (Herbst, R. S., and Shin, D. M., Cancer 94 (2002) 1593-1611; Mendelsohn, J., and Baselga, J., Oncogene 19 (2000) 6550-6565). EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay, G., et al., Ann. Oncology 14 (2003) 1346-1363; Tsao, A. S., and Herbst, R. S., Signal 4 (2003) 4-9; Herbst, R. S., and Shin, D. M., Cancer 94 (2002) 1593-1611; Modjtahedi, H., et al., Br. J. Cancer 73 (1996) 228-235).

Insulin-like growth factor I receptor (IGF-IR, CD 221 antigen) belongs to the family of transmembrane protein tyrosine kinases (LeRoith, D., et al., Endocrin. Rev. 16 (1995) 143-163; and Adams, T. E., et al., Cell. Mol. Life. Sci. 57 (2000) 1050-1063). SwissProt database entry P08069 provides the sequence of IGF-1R. IGF-IR binds IGF-I with high affinity and initiates the physiological response to this ligand in vivo. IGF-IR also binds to IGF-II, however with slightly lower affinity. IGF-IR overexpression promotes the neoplastic transformation of cells and there exists evidence that IGF-IR is involved in malignant transformation of cells and is therefore a useful target for the development of therapeutic agents for the treatment of cancer (Adams, T. E., et al., Cell. Mol. Life. Sci. 57 (2000) 1050-1063).

In one embodiment of the invention the bispecific antibody comprises a full length parent antibody as scaffold.

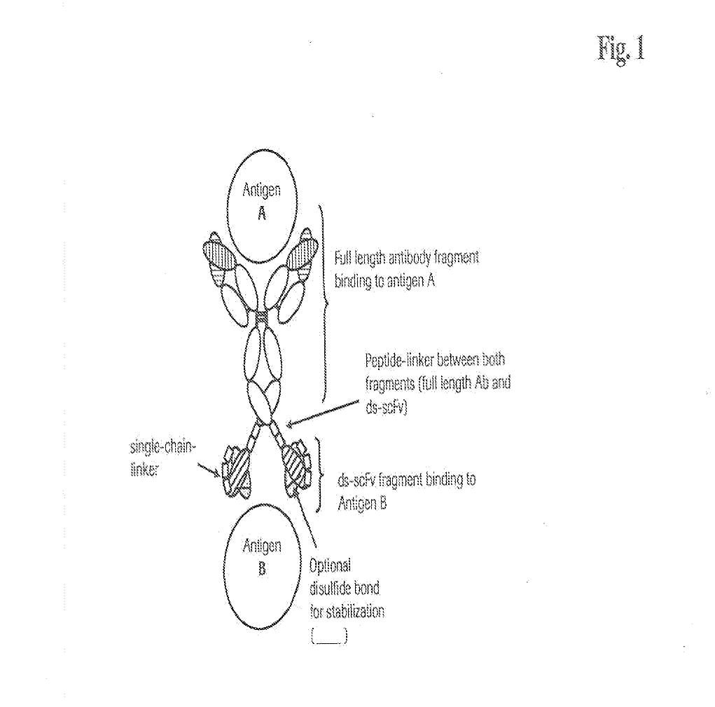

The term “full length antibody” denotes an antibody consisting of two “full length antibody heavy chains” and two “full length antibody light chains” (see FIG. 10 for schematic structure of a “full length antibody” without CH4 domain. See also in FIGS. 1 and 12 the full length part of tetravalent bispecific formats with single chain Fv attachments (XGFR) and with single chain Fab attachments (scFab-XGFR)). A “full length antibody heavy chain” is a polypeptide consisting in N-terminal to C-terminal direction of an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CH1, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be κ (kappa) or λ (lambda). The two full length antibody chains are linked together via inter-polypeptide disulfide bonds between the CL domain and the CH1 domain and between the hinge regions of the full length antibody heavy chains. Examples of typical full length antibodies are natural antibodies like IgG (e.g. IgG 1 and IgG2), IgM, IgA, IgD, and IgE. The full length antibodies according to the invention can be from a single species e.g. human, or they can be chimerized or humanized antibodies. The full length antibodies according to the invention comprise two antigen binding sites each formed by a pair of VH and VL, which both specifically bind to the same antigen. Thus a monospecific bivalent (=full length) antibody comprising a first antigen-binding site and consisting of two antibody light chains and two antibody heavy chains is a full length antibody. The C-terminus of the heavy or light chain of said full length antibody denotes the last amino acid at the C-terminus of said heavy or light chain. The N-terminus of the heavy or light chain of said full length antibody denotes the last amino acid at the N-terminus of said heavy or light chain.

In one embodiment said bispecific antibody is bivalent—using formats as described e.g. a) in WO 2009/080251, WO 2009/080252 or WO 2009/080253 (domain exchanged antibodies-see Example 14) or b) based on a scFab-Fc fusion antibody wherein one single chain Fab fragment is specific for EGFR and the other for IGF-1R (see Example 17) or c) in EP Appl. No. 07024867.9 (WO 2009/080251), Ridgway, J. B., Protein Eng. 9 (1996) 617-621; WO 96/027011; Merchant A. M, et al., Nature Biotech 16 (1998) 677-681; Atwell, S., et al., J. Mol. Biol. 270 (1997) 26-35 and EP 1870459A1. In one embodiment the bispecific antibody according to the invention is characterized in comprising as amino acid sequences of SEQ ID NO: 30, SEQ ID NO: 31, SEQ ID NO: 32 and SEQ ID NO: 33 or variants thereof. In one embodiment the bispecific antibody according to the invention is characterized in comprising as amino acid sequences of SEQ ID NO: 34, SEQ ID NO: 35, SEQ ID NO: 36 and SEQ ID NO: 37 or variants. In one embodiment the bispecific antibody according to the invention is characterized in comprising as amino acid sequences of SEQ ID NO: 38, and SEQ ID NO: 39 or variants thereof. In one embodiment the bispecific antibody according to the invention is characterized in comprising as amino acid sequences of SEQ ID NO: 40, and SEQ ID NO: 41 or variants thereof. In one embodiment the bispecific antibody according to the invention is characterized in comprising as amino acid sequences of SEQ ID NO: 42, and SEQ ID NO: 43 or variants thereof. These amino acid sequences are based on the heavy chain variable domains of SEQ ID NO: 8, and the light chain variable domains of SEQ ID NO: 10 (derived from humanized <EGFR>ICR62) as first antigen-binding site binding to EGFR, and on the heavy chain variable domains of SEQ ID NO: 23, and the light chain variable domains of SEQ ID NO: 25 (derived from the human anti-IGF-1R antibodies <IGF-1R> HUMAB Clone 18 (DSM ACC 2587)) as second antigen-binding site binding to IGF-1R.

In one embodiment said bispecific antibody is trivalent using e.g. formats based on a full length antibody specifically binding to one of the two receptors EGFR or IGF-1R, to which only at one C-terminus of one heavy chain a scFab fragment is fused which specifically binds to the other of the two receptors EGFR or IGF-1R, including knobs—into holes technology, as described e.g. in EP Appl. No 09004909.9 or e.g formats based on a full length antibody specifically binding to one of the two receptors EGFR or IGF-1R, to which at one C-terminus of one heavy chain a VH or VH-CH1 fragment and at the other C-terminus of the second heavy chain a VL or VL-CL fragment is fused which specifically binds to the other of the two receptors EGFR or IGF-1R, including knobs-into holes technology, as described e.g. in EP Appl. No 09005108.7. For the knobs intoholes technology and variations thereof see also Ridgway, J. B., Protein Eng. 9 (1996) 617-621; WO 96/027011, Merchant A. M, et al., Nature Biotech 16 (1998) 677-681; Atwell, S., et al., J. Mol. Biol. 270 (1997) 26-35; and EP 1870459A1. In one embodiment the bispecific, trivalent antibody according to the invention is characterized in comprising as amino acid sequences of SEQ ID NO: 44, SEQ ID NO: 45, and SEQ ID NO: 46 or variants thereof. In one embodiment the bispecific, trivalent antibody according to the invention is characterized in comprising as amino acid sequences of SEQ ID NO: 47, SEQ ID NO: 48, and SEQ ID NO: 49 or variants thereof. These amino acid sequences are based on the heavy chain variable domains of SEQ ID NO: 8, and the light chain variable domains of SEQ ID NO: 10 (derived from humanized <EGFR>ICR62) as first antigen-binding site binding to EGFR, and on the heavy chain variable domains of SEQ ID NO: 23, and the light chain variable domains of SEQ ID NO: 25 (derived from the human anti-IGF-1R antibodies <IGF-1R> HUMAB Clone 18 (DSM ACC 2587)) as second antigen-binding site binding to IGF-1R.

In one embodiment said bispecific antibody is tetravalent using formats as described e.g. in WO 2007/024715, or WO 2007/109254 or EP Appl. No 09004909.9 (full length antibody binding to first antigen to which two scFab fragments binding to the other antigen are fused) (see e.g. Examples 1 or 9).

In one embodiment said bispecific antibody is tetravalent, and consists of

a) a monospecific bivalent antibody comprising said first antigen-binding site and consisting of two antibody light chains and two antibody heavy chains each chain comprising only one variable domain,

b) two peptide-linkers, and

c) two monovalent monospecific single chain antibodies (monospecific monovalent single chain Fv) comprising said second antigen-binding site, each consisting of a light chain variable domain, a heavy chain variable domain, and an single-chain-linker between said light chain variable domain and said heavy chain variable domain;

wherein said single chain antibodies (said single chain Fv) are linked to the same terminus of the monospecific bivalent antibody light chains or the antibody heavy chains.

In another embodiment said bispecific antibody is tetravalent, and consists of

a) a monospecific bivalent antibody comprising said second antigen-binding site and consisting of two antibody light chains and two antibody heavy chains each chain comprising only one variable domain,

b) two peptide-linkers, and

c) two monovalent monospecific single chain antibodies (monospecific monovalent single chain Fv) comprising said first antigen-binding site, each consisting of a light chain variable domain, a heavy chain variable domain, and an single-chain-linker between said light chain variable domain and said heavy chain variable domain;

wherein said single chain antibodies (said single chain Fv) are linked to the same terminus of the monospecific bivalent antibody light chains or the antibody heavy chains.

In another embodiment said bispecific antibody is tetravalent, and consists of

a) a full length antibody comprising said antigen-binding site and consisting of two antibody heavy chains and two antibody light chains; and

b) two identical single chain Fab fragments comprising said second antigen-binding site, wherein said single chain Fab fragments under b) are fused to said full length antibody under a) via a peptide connector at the C- or N-terminus of the heavy or light chain of said full length antibody.

In another embodiment said bispecific antibody is tetravalent, and consists of

a) a full length antibody comprising said second antigen-binding site and consisting of two antibody heavy chains and two antibody light chains; and
b) two identical single chain Fab fragments comprising said first antigen-binding site, wherein said single chain Fab fragments under b) are fused to said full length antibody under a) via a peptide connector at the C- or N-terminus of the heavy or light chain of said full length antibody.

Preferably said single chain Fab fragments under b) are fused to said full length antibody under a) via a peptide connector at the C-terminus of the heavy or light chain of said full length antibody.

In one embodiment two identical single chain Fab fragments binding to a second antigen are fused to said full length antibody via a peptide connector at the C-terminus of each heavy or light chain of said full length antibody.

In one embodiment two identical single chain Fab fragments binding to a second antigen are fused to said full length antibody via a peptide connector at the C-terminus of each heavy chain of said full length antibody.

In one embodiment two identical single chain Fab fragments binding to a second antigen are fused to said full length antibody via a peptide connector at the C-terminus of each light chain of said full length antibody.

In a further embodiment said tetravalent bispecific antibody which has the following characteristics:

it is consisting of:

a) a monospecific bivalent parent (full length) antibody consisting of two full length antibody heavy chains and two full length antibody light chains whereby each chain is comprising only one variable domain,
b) two peptide-linkers,
c) two monospecific monovalent single chain antibodies (monospecific monovalent single chain Fv) each consisting of an antibody heavy chain variable domain, an antibody light chain variable domain, and a single-chain-linker between said antibody heavy chain variable domain and said antibody light chain variable domain;
and preferably said single chain antibodies (said single chain Fv) are linked to the same terminus (C- and N-terminus) of the monospecific bivalent antibody heavy chains or, alternatively to the same terminus (preferably the C-terminus) of the monospecific bivalent antibody light chains, and more preferably to the same terminus (C- and N-terminus) of the monospecific bivalent antibody heavy chains.

The term “peptide-linker” as used within the invention denotes a peptide with amino acid sequences, which is preferably of synthetic origin. These peptide-linkers according to invention are used to link the different antigen-binding sites and/or antibody fragments eventually comprising the different antigen-binding sites (e.g. single chain Fv, full length antibodies, a VH domain and/or a VL domain, Fab, (Fab)₂, Fc part) together to form a bispecific antibody according to the invention The peptide-linkers can comprise one or more of the following amino acid sequences listed in Table 1 as well as further arbitrarily selected amino acids. Said peptide-linkers are peptides with an amino acid sequence with a length of at least 5 amino acids, preferably of at least 10 amino acids, more preferably with a length between 10 and 50 amino acids. Preferably said peptide-linkers under b) are peptides with an amino acid sequence with a length of at least 10 amino acids. In one embodiment said peptide-linker is (GxS)n with G=glycine, S=serine, (x=3 and n=3, 4, 5 or 6) or (x=4 and n=2, 3, 4 or 5), preferably x=4 and n=2 or 3, more preferably with x=4, n=2 ((G₄S)₂). To said (GxS)n peptide-linker also additional G=glycines can be added, e.g. GG, or GGG.

The term “single-chain-linker” as used within the invention denotes a peptide with amino acid sequences, which is preferably of synthetic origin. These single-chain-linkers according to invention are used to link a VH and a VL domain to form a single chain Fv. Preferably the said single-chain-linker under c) is a peptide with an amino acid sequence with a length of at least 15 amino acids, more preferably with a length of at least 20 amino acids. In one embodiment said single-chain-linker is (GxS)n with G=glycine, S=serine, (x=3 and n=4, 5 or 6) or (x=4 and n=3, 4 or 5), preferably with x=4, n=4 or 5, more preferably with x=4, n=4.

Furthermore said single chain (single chain Fv) antibodies are preferably disulfide stabilized. Such further disulfide stabilization of single chain antibodies is achieved by the introduction of a disulfide bond between the variable domains of the single chain antibodies and is described e.g in WO 94/029350, Rajagopal, V., et al., Prot. Engin. 10 (12) (1997) 1453-59; Kobayashi, H., et al., Nuclear Medicine & Biology 25 (1998) 387-393; or Schmidt, M., et al., Oncogene 18 (1999) 1711-1721.

In one embodiment of the disulfide stabilized single chain (single chain Fv) antibodies, the disulfide bond between the variable domains of the single chain antibodies comprised in the antibody according to the invention is independently for each single chain antibody selected from:

i) heavy chain variable domain position 44 to light chain variable domain position 100,
ii) heavy chain variable domain position 105 to light chain variable domain position 43, or
iii) heavy chain variable domain position 101 to light chain variable domain position 100.

In one embodiment the disulfide bond between the variable domains of the single chain antibodies comprised in the antibody according to the invention is between heavy chain variable domain position 44 and light chain variable domain position 100. In one embodiment the disulfide bond between the variable domains of the single chain antibodies comprised in the antibody according to the invention is between heavy chain variable domain position 105 and light chain variable domain position 43.

In one embodiment said single chain (single chain Fv) antibodies without said optional disulfide stabilization between the variable domains VH and VL of the single chain antibody (single chain Fv) are preferred.

In a further embodiment the bispecific antibody is characterized by

two antigen-binding sites are each formed by the two pairs of heavy and light chain variable domains of the monospecific bivalent parent antibody and both bind to the same epitope,

the additional two antigen-binding sites are each formed by the heavy and light chain variable domain of one single chain antibody,

the single chain antibodies are each linked to one heavy chain or to one light chain via a peptide-linker, whereby each antibody chain terminus is linked only to a single chain antibody.

In a further embodiment said tetravalent bispecific antibody is characterized in that said monospecific bivalent (full length) antibody part under a) binds to EGFR and said two monovalent monospecific single chain antibodies under c) bind to IGF-1R.

In a further embodiment said tetravalent bispecific antibody is characterized in that said monospecific bivalent (full length) antibody part under a) binds to IGF-1R and said two monovalent monospecific single chain antibodies under c) bind to EGFR.

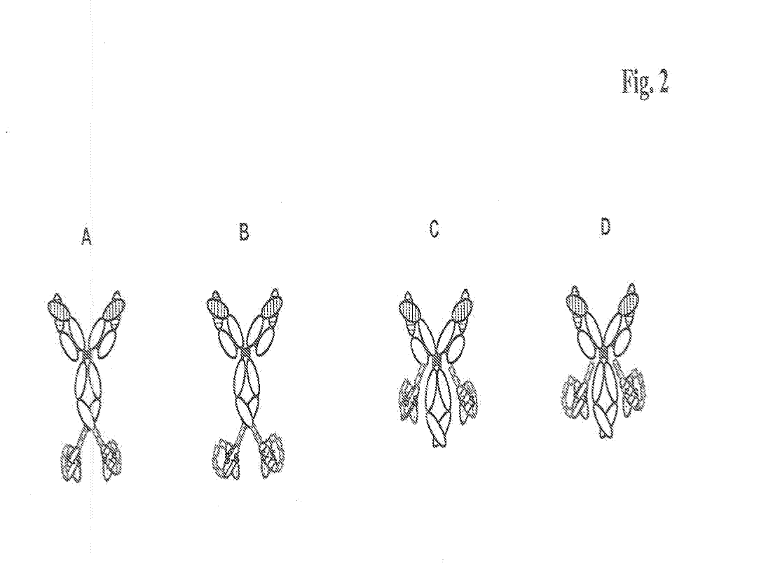

The structure of this first tetravalent embodiment of a bispecific antibody according to the invention binding to EGFR and IGF-1R, wherein one of the Antigens A or B is EGFR, while the other is IGF-1R. The structure is based on a full length antibody binding to Antigen A, to which two (optionally disulfide-stabilized) single chain Fv's binding to Antigen B, are linked via the a peptide-linker is exemplified in the schemes of FIGS. 1 and 2.

In a second tetravalent embodiment, the tetravalent, bispecific antibody comprises

a) a full length antibody specifically binding to said first antigen (one of the two antigens EGFR or IGF-1R) and consisting of two antibody heavy chains and two antibody light chains; and
b) two identical single chain Fab fragments specifically binding to said second antigen (the other of the two antigens EGFR or IGF-1R),
wherein said single chain Fab fragments under b) are fused to said full length antibody under a) via a peptide connector at the C- or N-terminus of the heavy or light chain of said full length antibody.

In one embodiment two identical single chain Fab fragments binding to a second antigen are fused to said full length antibody via a peptide connector at the C-terminus of each heavy or light chain of said full length antibody.

In one embodiment two identical single chain Fab fragments binding to a second antigen are fused to said full length antibody via a peptide connector at the C-terminus of each heavy chain of said full length antibody.

In one embodiment two identical single chain Fab fragments binding to a second antigen are fused to said full length antibody via a peptide connector at the C-terminus of each light chain of said full length antibody.

A “single chain Fab fragment” (see FIG. 11) is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments a) VH-CH1-linker-VL-CL, b) VL-CL-linker-VH-CH1, c) VH-CL-linker-VL-CH1 and d) VL-CH1-linker-VH-CL, are stabilized via the natural disulfide bond between the CL domain and the CH1 domain. The term “N-terminus” denotes the last amino acid of the N-terminus. The term “C-terminus” denotes the last amino acid of the C-terminus.

In a preferred embodiment said antibody domains and said linker in said single chain Fab fragment have one of the following orders in N-terminal to C-terminal direction:

a) VH-CH1-linker-VL-CL, or b) VL-CL-linker-VH-CH1, more preferably VL-CL-linker-VH-CH1.

In another preferred embodiment said antibody domains and said linker in said single chain Fab fragment have one of the following orders in N-terminal to C-terminal direction:

a) VH-CL-linker-VL-CH1 or b) VL-CH1-linker-VH-CL.

The term “peptide connector” as used within the invention denotes a peptide with amino acid sequences, which is preferably of synthetic origin. These peptide connectors according to invention are used to fuse the single chain Fab fragments to the C- or N-terminus of the full length antibody to form a multispecific antibody according to the invention. Preferably said peptide connectors under b) are peptides with an amino acid sequence with a length of at least 5 amino acids, preferably with a length of 5 to 100, more preferably of 10 to 50 amino acids. In one embodiment said peptide connector is (GxS)n or (GxS)nGm with G=glycine, S=serine, and (x=3, n=3, 4, 5 or 6, and m=0, 1, 2 or 3) or (x=4, n=2, 3, 4 or 5 and m=0, 1, 2 or 3), preferably x=4 and n=2 or 3, more preferably with x=4, n=2. In one embodiment said peptide connector is (G₄S)₂.

The term “linker” as used within the invention denotes a peptide with amino acid sequences, which is preferably of synthetic origin. These peptides according to invention are used to link a) VH-CH1 to VL-CL, b) VL-CL to VH-CH1, c) VH-CL to VL-CH1 or d) VL-CH1 to VH-CL to form the following single chain Fab fragments according to the invention a) VH-CH1-linker-VL-CL, b) VL-CL-linker-VH-CH1, c) VH-CL-linker-VL-CH1 or d) VL-CH1-linker-VH-CL. Said linker within the single chain Fab fragments is a peptide with an amino acid sequence with a length of at least 30 amino acids, preferably with a length of 32 to 50 amino acids. In one embodiment said linker is (GxS)n with G=glycine, S=serine, (x=3, n=8, 9 or 10 and m=0, 1, 2 or 3) or (x=4 and n=6, 7 or 8 and m=0, 1, 2 or 3), preferably with x=4, n=6 or 7 and m=0, 1, 2 or 3, more preferably with x=4, n=7 and m=2. In one embodiment said linker is (G₄S)₆G₂.

Optionally in said single chain Fab fragment, additionally to the natural disulfide bond between the CL-domain and the CH1 domain, also the antibody heavy chain variable domain (VH) and the antibody light chain variable domain (VL) are disulfide stabilized by introduction of a disulfide bond between the following positions:

i) heavy chain variable domain position 44 to light chain variable domain position 100,
ii) heavy chain variable domain position 105 to light chain variable domain position 43, or
iii) heavy chain variable domain position 101 to light chain variable domain position 100 (numbering always according to EU index of Kabat).

Such further disulfide stabilization of single chain Fab fragments is achieved by the introduction of a disulfide bond between the variable domains VH and VL of the single chain Fab fragments. Techniques to introduce unnatural disulfide bridges for stabilization for a single chain Fv are described e.g. in WO 94/029350, Rajagopal, V., et al, Prot. Engin. (1997) 1453-59; Kobayashi, H., et al; Nuclear Medicine & Biology, Vol. 25, (1998) 387-393; or Schmidt, M., et al, Oncogene (1999) 18, 1711-1721. In one embodiment the optional disulfide bond between the variable domains of the single chain Fab fragments comprised in the antibody according to the invention is between heavy chain variable domain position 44 and light chain variable domain position 100. In one embodiment the optional disulfide bond between the variable domains of the single chain Fab fragments comprised in the antibody according to the invention is between heavy chain variable domain position 105 and light chain variable domain position 43 (numbering always according to EU index of Kabat).

In an embodiment single chain Fab fragment without said optional disulfide stabilization between the variable domains VH and VL of the single chain Fab fragments are preferred.

Preferably said second embodiment of an tetravalent bispecific antibody according to the invention comprises two identical single chain Fab fragments (preferably VL-CL-linker-VH-CH1) which are both fused to the two C-termini of the two heavy chains or to the two C-termini of the two light chains of said full length antibody under a). Such fusion results in two identical fusion peptides (either i) heavy chain and single chain Fab fragment or ii) light chain and single chain Fab fragment) which are coexpressed with either i) the light chain or the heavy chain of the full length antibody to give the bispecific antibody according to the invention (see FIGS. 12, 13 and 14).

In a further embodiment said tetravalent bispecific antibody is characterized in that said full length antibody part under a) binds to EGFR and said two single chain Fab fragments under b) bind to IGF-1R.

In a further embodiment said tetravalent bispecific antibody is characterized in that said full length antibody part under a) binds to IGF-1R and said two single chain Fab fragments under b) bind to EGFR.

In a further embodiment said bispecific antibody is characterized in that the constant region derived of human origin.

In a further embodiment said bispecific antibody is characterized in that the constant region of the bispecific antibody according to the invention is of human IgG1 subclass, or of human IgG1 subclass with the mutations L234A and L235A.

In a further embodiment said bispecific antibody is characterized in that the constant region of the bispecific antibody according to the invention antibody is of human IgG2 subclass.

In a further embodiment said bispecific antibody is characterized in that the constant region of the bispecific antibody according to the invention antibody is of human IgG3 subclass.

In a further embodiment said bispecific antibody is characterized in the constant region of the bispecific antibody according to the invention is of human IgG4 subclass or, of IgG4 subclass with the additional mutation S228P.

It has now been found that the bispecific antibodies according to the current invention have improved characteristics. They show at least the same or increased in vitro and in vivo antitumor activity/efficacy compared to the application of only one or of two individual antibodies in combination, or to the bispecific antibodies of Lu, D., et al., Biochemical and Biophysical Research Communications 318 (2004) 507-513; J. Biol. Chem., 279 (2004) 2856-2865; and J. Biol. Chem. 280 (2005) 19665-72. They show an improved pharmacokinetic stability in vivo compared to the bispecific the bispecific antibodies of Lu, D., et al., Biochemical and Biophysical Research Communications 318 (2004) 507-513; J. Biol. Chem., 279 (2004) 2856-2865; and J. Biol. Chem. 280 (2005) 19665-72. Furthermore the bispecific antibodies according to the current invention show modulated receptor downregulation/internalization compared to the application of only one or of two individual antibodies in combination. Furthermore the bispecific antibodies according to the current invention may provide benefits such as reduced dose and/or frequency of administration and concomitantly cost savings.

The term “constant region” as used within the current applications denotes the sum of the domains of an antibody other than the variable region. The constant region is not involved directly in binding of an antigen, but exhibits various effector functions. Depending on the amino acid sequence of the constant region of their heavy chains, antibodies are divided in the classes: IgA, IgD, IgE, IgG and IgM, and several of these may be further divided into subclasses, such as IgG1, IgG2, IgG3, and IgG4, IgA1 and IgA2. The heavy chain constant regions that correspond to the different classes of antibodies are called α, δ, ε, γ, and μ, respectively. The light chain constant regions which can be found in all five antibody classes are called κ (kappa) and λ (lambda).

The term “constant region derived from human origin” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., (see e.g. Johnson, G. and Wu, T. T., Nucleic Acids Res. 28 (2000) 214-218; Kabat, E. A., et al., Proc. Natl. Acad. Sci. USA 72 (1975) 2785-2788). While antibodies of the IgG4 subclass show reduced Fc receptor (FcγRIIIa) binding, antibodies of other IgG subclasses show strong binding. However Pro238, Asp265, Asp270, Asn297 (loss of Fc carbohydrate), Pro329, Leu234, Leu235, Gly236, Gly237, Ile253, Ser254, Lys288, Thr307, Gln311, Asn434, and His435 are residues which, if altered, provide also reduced Fc receptor binding (Shields, R., L., et al., J. Biol. Chem. 276 (2001) 6591-6604; Lund, J., et al., FASEB J. 9 (1995) 115-119; Morgan, A., et al., Immunology 86 (1995) 319-324; EP 0 307 434). In one embodiment an antibody according to the invention has a reduced FcR binding compared to an IgG1 antibody and the monospecific bivalent (full length) parent antibody is in regard to FcR binding of IgG4 subclass or of IgG1 or IgG2 subclass with a mutation in 5228, L234, L235 and/or D265, and/or contains the PVA236 mutation. In one embodiment the mutations in the monospecific bivalent (full length) parent antibody are S228P, L234A, L235A, L235E and/or PVA236. In another embodiment the mutations in the monospecific bivalent (full length) parent antibody are in IgG4 S228P and in IgG1 L234A and L235A. Constant heavy chain regions shown in SEQ ID NO: 27 and 28. In one embodiment the constant heavy chain region of the monospecific bivalent (full length) parent antibody is of SEQ ID NO: 27 with mutations L234A and L235A. In another embodiment the constant heavy chain region of the monospecific bivalent (full length) parent antibody is of SEQ ID NO: 28 with mutation S228P. In another embodiment the constant light chain region of the monospecific bivalent (full length) parent antibody is of SEQ ID NO: 29.

The constant region of an antibody is directly involved in ADCC (antibody-dependent cell-mediated cytotoxicity) and CDC (complement-dependent cytotoxicity). Complement activation (CDC) is initiated by binding of complement factor C1q to the constant region of most IgG antibody subclasses. Binding of C1q to an antibody is caused by defined protein-protein interactions at the so called binding site. Such constant region binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such constant region binding sites are, e.g., characterized by the amino acids L234, L235, D270, N297, E318, K320, K322, P331, and P329 (numbering according to EU index of Kabat).

The term “antibody-dependent cellular cytotoxicity (ADCC)” refers to lysis of human target cells by an antibody according to the invention in the presence of effector cells. ADCC is measured preferably by the treatment of a preparation of IGF-1R and EGFR expressing cells with an antibody according to the invention in the presence of effector cells such as freshly isolated PBMC or purified effector cells from buffy coats, like monocytes or natural killer (NK) cells or a permanently growing NK cell line.

The term “complement-dependent cytotoxicity (CDC)” denotes a process initiated by binding of complement factor C1q to the Fc part of most IgG antibody subclasses. Binding of C1q to an antibody is caused by defined protein-protein interactions at the so called binding site. Such Fc part binding sites are known in the state of the art (see above). Such Fc part binding sites are, e.g., characterized by the amino acids L234, L235, D270, N297, E318, K320, K322, P331, and P329 (numbering according to EU index of Kabat). Antibodies of subclass IgG1, IgG2, and IgG3 usually show complement activation including C1q and C3 binding, whereas IgG4 does not activate the complement system and does not bind C1q and/or C3.

Cell-mediated effector functions of monoclonal antibodies can be enhanced by engineering their oligosaccharide component as described in Umana, P., et al., Nature Biotechnol. 17 (1999) 176-180, and U.S. Pat. No. 6,602,684. IgG1 type antibodies, the most commonly used therapeutic antibodies, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A., and Morrison, S. L., Trends Biotechnol. 15 (1997) 26-32). Umana, P., et al. Nature Biotechnol. 17 (1999) 176-180 and WO 99/54342 showed that overexpression in Chinese hamster ovary (CHO) cells of β(1,4)-N-acetylglucosaminyltransferase III (“GnTIII”), a glycosyltransferase catalyzing the formation of bisected oligosaccharides, significantly increases the in vitro ADCC activity of antibodies. Alterations in the composition of the Asn297 carbohydrate or its elimination affect also binding to FcγR and C1q (Umana, P., et al., Nature Biotechnol. 17 (1999) 176-180; Davies, J., et al., Biotechnol. Bioeng. 74 (2001) 288-294; Mimura, Y., et al., J. Biol. Chem. 276 (2001) 45539-45547; Radaev, S., et al., J. Biol. Chem. 276 (2001) 16478-16483; Shields, R. L., et al., J. Biol. Chem. 276 (2001) 6591-6604; Shields, R. L., et al., J. Biol. Chem. 277 (2002) 26733-26740; Simmons, L. C., et al., J. Immunol. Methods 263 (2002) 133-147).

Methods to enhance cell-mediated effector functions of monoclonal antibodies are reported e.g. in WO 2005/044859, WO 2004/065540, WO2007/031875, Umana, P., et al., Nature Biotechnol. 17:176-180 (1999), WO 99/154342, WO 2005/018572, WO 2006/116260, WO 2006/114700, WO 2004/065540, WO 2005/011735, WO 2005/027966, WO 1997/028267, US 2006/0134709, US 2005/0054048, US 2005/0152894, WO 2003/035835 and WO 2000/061739 or e.g. in Niwa, R., et al., J. Immunol. Methods 306 (2005) 151-160; Shinkawa, T., et al, J Biol Chem, 278 (2003) 3466-3473; WO 03/055993 and US2005/0249722.

Therefore in one embodiment of the invention, the bispecific antibody is glycosylated (if it comprises an Fc part of IgG1, IgG2, IgG3 or IgG4 subclass, preferably of IgG1 or IgG3 subclass) with a sugar chain at Asn297 whereby the amount of fucose within said sugar chain is 65% or lower (Numbering according to Kabat). In another embodiment is the amount of fucose within said sugar chain is between 5% and 65%, preferably between 20% and 40%. “Asn297” according to the invention means amino acid asparagine located at about position 297 in the Fc region. Based on minor sequence variations of antibodies, Asn297 can also be located some amino acids (usually not more than +3 amino acids) upstream or downstream of position 297, i.e. between position 294 and 300. In one embodiment the glycosylated antibody according to the invention the IgG subclass is of human IgG1 subclass, of human IgG1 subclass with the mutations L234A and L235A or of IgG3 subclass. In a further embodiment the amount of N-glycolylneuraminic acid (NGNA) is 1% or less and/or the amount of N-terminal alpha-1,3-galactose is 1% or less within said sugar chain. The sugar chain show preferably the characteristics of N-linked glycans attached to Asn297 of an antibody recombinantly expressed in a CHO cell.

The term “the sugar chains show characteristics of N-linked glycans attached to Asn297 of an antibody recombinantly expressed in a CHO cell” denotes that the sugar chain at Asn297 of the constant region of the bispecific antibody according to the invention has the same structure and sugar residue sequence except for the fucose residue as those of the same antibody expressed in unmodified CHO cells, e.g. as those reported in WO 2006/103100.

The term “NGNA” as used within this application denotes the sugar residue N-glycolylneuraminic acid.

Glycosylation of human IgG1 or IgG3 occurs at Asn297 as core fucosylated biantennary complex oligosaccharide glycosylation terminated with up to two Gal residues. Human constant heavy chain regions of the IgG1 or IgG3 subclass are reported in detail by Kabat, E. A., et al., Sequences of Proteins of Immunological Interest, 5th Ed. Public Health Service, National Institutes of Health, Bethesda, Md. (1991), and by Brüggemann, M., et al., J. Exp. Med. 166 (1987) 1351-1361; Love, T. W., et al., Methods Enzymol. 178 (1989) 515-527. These structures are designated as G0, G1 (α-1,6- or α-1,3-), or G2 glycan residues, depending from the amount of terminal Gal residues (Raju, T. S., Bioprocess Int. 1 (2003) 44-53). CHO type glycosylation of antibody Fc parts is e.g. described by Routier, F. H., Glycoconjugate J. 14 (1997) 201-207. Antibodies which are recombinantly expressed in non-glycomodified CHO host cells usually are fucosylated at Asn297 in an amount of at least 85%. The modified oligosaccharides of the constant region of the bispecific antibody according to the invention may be hybrid or complex. Preferably the bisected, reduced/not-fucosylated oligosaccharides are hybrid. In another embodiment, the bisected, reduced/not-fucosylated oligosaccharides are complex.

According to the invention “amount of fucose” means the amount of said sugar within the sugar chain at Asn297, related to the sum of all glycostructures attached to Asn297 (e.g. complex, hybrid and high mannose structures) measured by MALDI-TOF mass spectrometry and calculated as average value. The relative amount of fucose is the percentage of fucose-containing structures related to all glycostructures identified in an N-Glycosidase F treated sample (e.g. complex, hybrid and oligo- and high-mannose structures, resp.) by MALDI-TOF.

For all bispecific antibodies according to the invention, “GE” means glycoengineered.

In one further aspect of the invention the bispecific antibody according to the invention is an antibody with ADCC and/or CDC, and has a constant region of IgG1 or IgG3 (preferably IgG1) subclass from human origin which does bind Fcγ receptor and/or complement factor C1q. Such an antibody which does bind Fc receptor and/or complement factor C1q does elicit antibody-dependent cellular cytotoxicity (ADCC) and/or complement dependent cytotoxicity (CDC).

The antibody according to the invention is produced by recombinant means. Thus, one aspect of the current invention is a nucleic acid encoding the antibody according to the invention and a further aspect is a cell comprising said nucleic acid encoding an antibody according to the invention. Methods for recombinant production are widely known in the state of the art and comprise protein expression in prokaryotic and eukaryotic cells with subsequent isolation of the antibody and usually purification to a pharmaceutically acceptable purity. For the expression of the antibodies as aforementioned in a host cell, nucleic acids encoding the respective modified light and heavy chains are inserted into expression vectors by standard methods. Expression is performed in appropriate prokaryotic or eukaryotic host cells like CHO cells, NS0 cells, SP2/0 cells, HEK293 cells, COS cells, PER.C6 cells, yeast, or E. coli cells, and the antibody is recovered from the cells (supernatant or cells after lysis). General methods for recombinant production of antibodies are well-known in the state of the art and described, for example, in the review articles of Makrides, S. C., Protein Expr. Purif 17 (1999) 183-202; Geisse, S., et al., Protein Expr. Puff. 8 (1996) 271-282; Kaufman, R. J., Mol. Biotechnol. 16 (2000) 151-160; Werner, R. G., Drug Res. 48 (1998) 870-880.

The bispecific antibodies are suitably separated from the culture medium by conventional immunoglobulin purification procedures such as, for example, protein A-Sepharose, hydroxylapatite chromatography, gel electrophoresis, dialysis, or affinity chromatography. DNA and RNA encoding the monoclonal antibodies is readily isolated and sequenced using conventional procedures. The hybridoma cells can serve as a source of such DNA and RNA. Once isolated, the DNA may be inserted into expression vectors, which are then transfected into host cells such as HEK 293 cells, CHO cells, or myeloma cells that do not otherwise produce immunoglobulin protein, to obtain the synthesis of recombinant monoclonal antibodies in the host cells.

Amino acid sequence variants (or mutants) of the bispecific antibody are prepared by introducing appropriate nucleotide changes into the antibody DNA, or by nucleotide synthesis. Such modifications can be performed, however, only in a very limited range, e.g. as described above. For example, the modifications do not alter the above mentioned antibody characteristics such as the IgG isotype and antigen binding, but may improve the yield of the recombinant production, protein stability or facilitate the purification.

The bispecific antibodies binding to EGFR and IGF-1R according to the invention downregulate EGFR. In one embodiment, the downregulation of EGFR is at least about 30%, in another embodiment at least about 35%, and in still another embodiment at least about 40% in A549 cells.

The bispecific antibodies binding to EGFR and IGF-1R according to the invention downregulate IGF-1R. In one embodiment, the downregulation of IGF-1R by the bispecific Cross-Mab (VH/VL) or Cross-Mab (CH/CL) is at most about 15%, in another embodiment at most about 20%, and in still another embodiment at least most 40% in H322M cells (at 75 μg Protein/mL).

The term “host cell” as used in the current application denotes any kind of cellular system which can be engineered to generate the antibodies according to the current invention. In one embodiment HEK293 cells and CHO cells are used as host cells. As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included.

Expression in NS0 cells is described by, e.g., Barnes, L. M., et al., Cytotechnology 32 (2000) 109-123; Barnes, L. M., et al., Biotech. Bioeng. 73 (2001) 261-270. Transient expression is described by, e.g., Durocher, Y., et al., Nucl. Acids. Res. 30 E9 (2002). Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; and Norderhaug, L., et al., J. Immunol. Methods 204 (1997) 77-87. A preferred transient expression system (HEK 293) is described by Schlaeger, E.-J., and Christensen, K., in Cytotechnology 30 (1999) 71-83 and by Schlaeger, E.- J., in J. Immunol. Methods 194 (1996) 191-199.

The control sequences that are suitable for prokaryotes, for example, include a promoter, optionally an operator sequence, and a ribosome binding site. Eukaryotic cells are known to utilize promoters, enhancers and polyadenylation signals.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

An antibody according to the invention with a reduced amount of fucose can be expressed in a glycomodified host cell engineered to express at least one nucleic acid encoding a polypeptide having GnTIII activity and a polypeptide having ManII activity in an amount to fucosylate according to the invention the oligosaccharides in the Fc region. In one embodiment, the polypeptide having GnTIII activity is a fusion polypeptide. Alternatively α1,6-fucosyltransferase activity of the host cell can be decreased or eliminated according to U.S. Pat. No. 6,946,292 to generate glycomodified host cells. The amount of antibody fucosylation can be predetermined e.g. either by fermentation conditions or by combination of at least two antibodies with different fucosylation amount.

The antibody according to the invention with a reduced amount of fucose can be produced in a host cell by a method comprising: (a) culturing a host cell engineered to express at least one polynucleotide encoding a fusion polypeptide having GnTIII activity and/or ManII activity under conditions which permit the production of said antibody and which permit fucosylation of the oligosaccharides present on the Fc region of said antibody in an amount according to the invention; and (b) isolating said antibody. In one embodiment, the polypeptide having GnTIII activity is a fusion polypeptide, preferably comprising the catalytic domain of GnTIII and the Golgi localization domain of a heterologous Golgi resident polypeptide selected from the group consisting of the localization domain of mannosidase II, the localization domain of β(1,2)-N-acetylglucosaminyltransferase I (“GnTI”), the localization domain of marmosidase I, the localization domain of β(1,2)-N-acetylglucosaminyltransferase II (“GnTII”), and the localization domain of α-1,6 core fucosyltransferase. Preferably, the Golgi localization domain is from mannosidase II or GnTI.

As used herein, a “polypeptide having GnTIII activity” refers to polypeptides that are able to catalyze the addition of an N-acetylglucosamine (GlcNAc) residue in β-1,4 linkage to the β-linked mannoside of the trimannosyl core of N-linked oligosaccharides. This includes fusion polypeptides exhibiting enzymatic activity similar to, but not necessarily identical to, an activity of β-1,4-N-acetylglucosaminyltransferase III, also known as β-1,4-mannosyl-glycoprotein 4-beta-N-acetylglucosaminyl-transferase (EC 2.4.1.144), according to the Nomenclature Committee of the International Union of Biochemistry and Molecular Biology (NC-IUBMB), as measured in a particular biological assay, with or without dose dependency. In the case where dose dependency does exist, it need not be identical to that of GnTIII, but rather substantially similar to the dose-dependence in a given activity as compared to the GnTIII (i.e. the candidate polypeptide will exhibit greater activity or not more than about 25-fold less and, preferably, not more than about tenfold less activity, and most preferably, not more than about three-fold less activity relative to the GnTIII). As used herein, the term “Golgi localization domain” refers to the amino acid sequence of a Golgi resident polypeptide which is responsible for anchoring the polypeptide in location within the Golgi complex. Generally, localization domains comprise amino terminal “tails” of an enzyme.

For the production of antibodies according to the invention with a reduce amount of fucose likewise a host cell that is able and engineered to allow the production of an antibody with modified glycoforms can be used. Such a host cell has been further manipulated to express increased levels of one or more polypeptides having GnTIII activity. CHO cells are preferred as such host cells. Likewise cells producing antibody compositions with increased ADCC as reported in U.S. Pat. No. 6,946,292.

Purification of antibodies is performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987). Different methods are well established and widespread used for protein purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M., A., Appl. Biochem. Biotech. 75 (1998) 93-102).

One aspect of the invention is a pharmaceutical composition comprising an antibody according to the invention. Another aspect of the invention is the use of an antibody according to the invention for the manufacture of a pharmaceutical composition. A further aspect of the invention is a method for the manufacture of a pharmaceutical composition comprising an antibody according to the invention. In another aspect, the present invention provides a composition, e.g. a pharmaceutical composition, containing an antibody according to the present invention, formulated together with a pharmaceutical carrier.

It has surprisingly been found that the bispecific antibody against EGFR and against IGF-1R according to the invention has improved anti-proliferative properties against cancer cells when compared to the monospecific parent anti-EGFR antibodies and anti-IGF-1R antibodies or compared to the bispecific antibodies against EGFR and against IGF-1R known from Lu, D., et al., Biochemical and Biophysical Research Communications 318 (2004) 507-513; J. Biol. Chem., 279 (2004) 2856-2865; and J. Biol. Chem. 280 (2005) 19665-72 (as these bispecific antibodies only show reduced efficacy in tumor cells which express EGFR/IGF-1R compared to the combination of the respective parent antibodies).

Another aspect of the invention is said pharmaceutical composition for the treatment of cancer.

Another aspect of the invention is the use of an antibody according to the invention for the manufacture of a medicament for the treatment of cancer.

Another aspect of the invention is method of treatment of patient suffering from cancer by administering an antibody according to the invention to a patient in the need of such treatment.

As used herein, “pharmaceutical carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion).

A composition of the present invention can be administered by a variety of methods known in the art. As will be appreciated by the skilled artisan, the route and/or mode of administration will vary depending upon the desired results. To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Pharmaceutically acceptable diluents include saline and aqueous buffer solutions. Pharmaceutical carriers include sterile aqueous solutions or dispersions and sterile powders for the extemporaneous preparation of sterile injectable solutions or dispersion. The use of such media and agents for pharmaceutically active substances is known in the art.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The term cancer as used herein refers to proliferative diseases, such as lymphomas, lymphocytic leukemias, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewing's sarcoma, including refractory versions of any of the above cancers, or a combination of one or more of the above cancers.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, the carrier preferably is an isotonic buffered saline solution.

Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Variant progeny that have the same function or biological activity as screened for in the originally transformed cell are included. Where distinct designations are intended, it will be clear from the context.

The term “transformation” as used herein refers to process of transfer of a vectors/nucleic acid into a host cell. If cells without formidable cell wall barriers are used as host cells, transfection is carried out e.g. by the calcium phosphate precipitation method as described by Graham, F. L., and Van der Eb, A. J., Virology 52 (1973) 456-467. However, other methods for introducing DNA into cells such as by nuclear injection or by protoplast fusion may also be used. If prokaryotic cells or cells which contain substantial cell wall constructions are used, e.g. one method of transfection is calcium treatment using calcium chloride as described by Cohen, S. N., et al, PNAS. 69 (1972) 2110-2114.

As used herein, “expression” refers to the process by which a nucleic acid is transcribed into mRNA and/or to the process by which the transcribed mRNA (also referred to as transcript) is subsequently being translated into peptides, polypeptides, or proteins. The transcripts and the encoded polypeptides are collectively referred to as gene product. If the polynucleotide is derived from genomic DNA, expression in a eukaryotic cell may include splicing of the mRNA.

A “vector” is a nucleic acid molecule, in particular self-replicating, which transfers an inserted nucleic acid molecule into and/or between host cells. The term includes vectors that function primarily for insertion of DNA or RNA into a cell (e.g., chromosomal integration), replication of vectors that function primarily for the replication of DNA or RNA, and expression vectors that function for transcription and/or translation of the DNA or RNA. Also included are vectors that provide more than one of the functions as described.

An “expression vector” is a polynucleotide which, when introduced into an appropriate host cell, can be transcribed and translated into a polypeptide. An “expression system” usually refers to a suitable host cell comprised of an expression vector that can function to yield a desired expression product.

The following examples, sequence listing and figures are provided to aid the understanding of the present invention, the true scope of which is set forth in the appended claims. It is understood that modifications can be made in the procedures set forth without departing from the spirit of the invention.

### Experimental Procedure

### EXAMPLES

### Design of Bispecific <EGFR-IGF-1R> Antibodies

The bispecific antibodies binding to EGFR and IGF-1R according to the invention comprise a first antigen-binding site that binds to EGFR and a second antigen-binding site that binds to IGF-1R. As first antigen-binding site binding to EGFR the heavy chain variable domains of SEQ ID NO: 7 or SEQ ID NO: 8, and the light chain variable domains of SEQ ID NO: 9 or SEQ ID NO: 10, which are both derived from the humanized rat anti-EGFR antibody ICR62 which is described in detail in WO 2006/082515, can be used.

As second antigen-binding site binding to IGF-1R comprises the heavy chain variable domains of SEQ ID NO: 23 or SEQ ID NO: 24, and the light chain variable domains of SEQ ID NO: 25 or SEQ ID NO: 26, which are both derived from the human anti-IGF-1R antibodies <IGF-1R> HUMAB Clone 18 (DSM ACC 2587) or <IGF-1R> HUMAB Clone 22 (DSM ACC 2594) which are described in detail in WO 2005/005635, can be used.

In all following Examples 1 to 20 the bispecific <EGFR-IGF-1R> antibodies were based on the heavy chain variable domains of SEQ ID NO: 8, and the light chain variable domains of SEQ ID NO: 10 (derived from humanized <EGFR>ICR62) as first antigen-binding site binding to EGFR, and on the heavy chain variable domains of SEQ ID NO: 23, and the light chain variable domains of SEQ ID NO: 25 (derived from the human anti-IGF-1R antibodies <IGF-1R> HUMAB Clone 18 (DSM ACC 2587)) as second antigen-binding site binding to IGF-1R.

A) Design of Bispecific <EGFR-IGF-1R> Antibodies with scFv Attachment (XGFR1 and XGFR2 Nomenclature which Refers to scFv-XGFR Molecules)

To generate agents that combine features of both antibodies, various novel tetravalent bispecific antibody-derived protein entities were constructed. In these molecules, recombinant single-chain binding molecules of one antibody are connected via recombinant protein fusion technologies to the other antibody which was retained in the format of a full-length IgG1. This second antibody carries the desired second binding specificity.

A summary of the designed formats based on the human anti-IGF-1R antibodies <IGF-1R> HUMAB Clone 18 (DSM ACC 2587) and the single chain Fv (scFv) binding to EGFR derived from the heavy chain variable domain (VH) of SEQ ID NO: 8, and the light chain variable domain (VL) of SEQ ID NO: 10 is shown in FIG. 1 and listed in Tables 1 and 2.

By gene synthesis and recombinant molecular biology techniques, the heavy chain variable domain (VH) of SEQ ID NO: 8, and the light chain variable domain (VL) of SEQ ID NO: 10 were linked by a glycine serine (G4S)n single-chain-linker to give a single chain Fv (scFv) binding to EGFR, which was attached to variable positions at either the N- or C-terminus of the anti-IGF-1R antibody <IGF-1R> HUMAB Clone 18 (DSM ACC 2587) light or heavy chain. In addition, cysteine residues were introduced at various positions in the VH (including Kabat position 44,) and VL (including Kabat position 100,) domain of the scFv binding to EGFR as described earlier (e.g. WO 94/029350; Reiter, Y., et al., Nature biotechnology 14 (1996) 1239-1245; Young, N., M., et al., FEBS Letters Vol. 377 (1995) 135-139; or Rajagopal, V., et al, Protein Engineering Vol. 10 1453-59 (1997). Subsequently, protein expression, stability and biological activity was evaluated. Furthermore, the (glycine-4-serine)n-containing peptide-linker length between the C-terminus of heavy or light chains of the <IGF1R> antibody and the scFv binding to EGFR was varied. Also, the length of the glycine-4-serine (G₄S) single-chain-linker that is an integral part of the single-chain Fv module binding to EGFR has been varied. All these molecules were recombinantly produced, purified and characterized. A summary of all bispecific antibody designs that were applied to generate tetravalent bispecific <EGFR-IGF1R> antibodies is given in Tables 1 and 2. For this study, we use the term ‘XGFR’ to describe the various protein entities that simultaneously recognize EGFR as well as IGF1R and comprise a full length antibody specifically binding to one of EGFR or IGF1R; and two scFv fragments specifically binding to the other of EGFR or IGF1R.

TABLE 1
The different bispecific <EGFR-IGF1R> antibody formats with N- and C-terminal
scFv attachments and the corresponding XGFR1-nomenclature and XGFR2-nomenclature.

| | Linking position of | Single-chain- | Peptide- | Cys-Positions |
|---|---|---|---|---|
| MoleculeName | scFv to the full | linker (G4S)n | linker(G4S)n | in the disulfide |
| XGFR-nomenclature | length antibody | n = | n = | stabilized scFv |
| anti-IGF-1R | — | — | — | — |
| antibody <IGF-1R> | | | | |
| HUMAB Clone 18 | | | | |
| humanized rat | — | — | — | — |
| anti-EGFR | | | | |
| antibody ICR62 | | | | |
| with VH of | | | | |
| SEQ ID NO: 8, | | | | |
| and VL of | | | | |
| SEQ ID NO: 10 | | | | |
| XGFR1-2320 | C-terminus HC | 3 | 2 | — |
| XGFR1-3320 | N-terminus HC | 3 | 2 | — |
| XGFR1-4320 | C-terminus LC | 3 | 2 | — |
| XGFR1-5320 | N-terminus LC | 3 | 2 | — |
| XGFR1-2321 | C-terminus HC | 3 | 2 | VH44/VL100 |
| XGFR1-3321 | N-terminus HC | 3 | 2 | VH44/VL100 |
| XGFR1-4321 | C-terminus LC | 3 | 2 | VH44/VL100 |
| XGFR1-5321 | N-terminus LC | 3 | 2 | VH44/VL100 |
| XGFR2-2420 | C-terminus HC | 4 | 2 | — |
| XGFR2-2421 | C-terminus HC | 4 | 2 | VH44/VL100 |
| XGFR2-3421 | N-terminus HC | 4 | 2 | VH44/VL100 |
| XGFR2-4421 | C-terminus LC | 4 | 2 | VH44/VL100 |
| XGFR2-5421 | N-terminus LC | 4 | 2 | VH44/VL100 |

The XGFR1 formats are based on a) the human anti-IGF-1R antibodies <IGF-1R> HUMAB Clone 18 (DSM ACC 2587) and b) two single chain Fv (scFv) binding to EGFR derived from the heavy chain variable domain (VH) of SEQ ID NO: 8, and the light chain variable domain (VL) of SEQ ID NO: 10, which are linked to the same terminus (C- or N-terminus) of either the heavy chain (HC) or the light chain (LC) of anti-IGF-1R antibodies <IGF-1R> HUMAB Clone 18.
In the XGFR2 formats are based on a) the variable regions VH (SEQ ID NO: 8) and VL (SEQ ID NO: 10) of humanized rat anti-EGFR antibody ICR62 and b) two single chain Fv (scFv) binding to human anti-IGF-1R antibodies <IGF-1R> HUMAB Clone 18 (DSM ACC 2587) VH (SEQ ID NO: 23) and VL (SEQ ID NO: 25) of anti-IGF-1R antibodies <IGF-1R> HUMAB Clone 18.
An “—” in the table means “not present”

TABLE 2
XGFR1 bispecific antibodies with variable single-chain-linker and
peptide-linker length.

| Molecule | Linking | Single- | Single- | | | Cys- |
|---|---|---|---|---|---|---|
| Name | position of | chain- | chain- | Peptide- | Peptide- | Positions in |
| XGFR- | scFv to the | linker | linker | linker | linker | the disulfide |
| nomen- | full length | (G4S)n | (G3S)n | (G4S)n | (G3S)n | stabilized |
| clature | antibody | n = | n = | n = | n = | scFv |
| XGFR1- | C-terminus | 4 | — | 2 | — | VH44/ |
| 2421 | HC | | | | | VL100 |
| XGFR1- | N-terminus | 4 | — | 2 | — | VH44/ |
| 3421 | HC | | | | | VL100 |
| XGFR1- | C-terminus | 4 | — | 2 | — | VH44/ |
| 4421 | LC | | | | | VL100 |
| XGFR1- | N-terminus | 4 | — | 2 | — | VH44/ |
| 5421 | LC | | | | | VL100 |
| XGFR1- | C-terminus | 4 | — | 5 | — | VH44/ |
| 2451 | HC | | | | | VL100 |
| XGFR1- | C-terminus | 4 | — | 5 | — | VH44/ |
| 4451 | HC | | | | | VL100 |
| XGFR1- | C-terminus | — | 5 | — | 3 | VH44/ |
| 2421C | HC | | | | | VL100 |

An “—” in the table means “not present”.

Examples 1 to 8 refer to the tetravalent XGFR1 and XGFR2 molecules with scFv attachment

B) Design of Tetravalent, Bispecific <EGFR-IGF-1R> Antibodies with Single Chain Fab (scFab) Attachment (scFab-XGFR1 and scFab-XGFR2 Nomenclature)

The term scFab-XGFR is used to describe the various protein entities that simultaneously recognize EGFR as well as IGF1R and comprise a full length antibody specifically binding to one of EGFR or IGF1R; and two scFab fragments specifically binding to the other of EGFR or IGF1R.

In the following as one embodiment of the invention tetravalent bispecific antibodies comprising a full length antibody binding to a first antigen (IGF-1R or EGFR) with two single chain Fab fragments binding to a second different antigen (the other of IGF-1R or EGFR) connected via peptide connector to the full length antibody (either both single chain Fab fragments at the two C-termini of the heavy chain or at the two C-termini of the light chain) are exemplified. The antibody domains and the linker in said single chain Fab fragment have the following order in N-terminal to C-terminal direction: VL-CL-linker-VH-CH1.

As heavy chain variable domain VH for the <IGF-1R> antigen binding site SEQ ID NO: 23 was used. As light chain variable domain VL for the <IGF-1R> antigen binding site SEQ ID NO: 25 was used.

As heavy chain variable domain VH for the <EGFR> antigen binding site SEQ ID NO: 8 was used. As light chain variable domain VL for the <EGFR> antigen binding site SEQ ID NO: 10 was used.

By gene synthesis and recombinant molecular biology techniques, VL-CL and VH-CH1, comprising the VH and VL of the respective antigen binding site were linked by a glycine serine (G4S)nGm linker to give a single chain Fab fragment VL-CL-linker-VH-CH1, which was attached to the C-terminus of the antibody heavy or light chain using (G4S)n peptide connector.

Optionally, cysteine residues were introduced in the VH (including Kabat position 44,) and VL (including Kabat position 100) domain of the single chain Fab fragment according to techniques as described earlier (e.g. WO 94/029350; Reiter, Y., et al., Nature biotechnology (1996) 1239-1245; Young, N. M., et al, FEBS Letters (1995) 135-139; or Rajagopal, V., et al, Protein Engineering (1997) 1453-59).

All these molecules were recombinantly produced, purified and characterized and protein expression, stability and biological activity was evaluated.

A summary of the antibody designs that were applied to generate tetravalent, bispecific scFab <EGFR-IGF-1R>, <IGF-1R-EGFR> antibodies is given in Table 3. For this study, we use the term ‘scFab-Ab’ to describe the various tetravalent protein entities. A representation of the designed formats is shown in FIGS. 13 and 14 and listed in Table 3.

TABLE 3
The different bispecific anti-IGF1R and anti-EGFR scFab-tetravalent antibody
formats with C- terminal single chain Fab fragment attachments and the corresponding scFab-
Ab-nomenclature. nomenclature

| Molecule | | | | | | | |
|---|---|---|---|---|---|---|---|
| Name | | | | | | | |
| (ScFab-Ab- | Full length | Single | Variable | Position of | | | scFab |
| nomen- | Antibody | chain Fab | Domains | single chain | | | disulfide |
| clature for | backbone | fragment | VH and | Fab | | | VH44/ |
| bispecific | derived | derived | VL: SEQ | attached to | | Peptide | VL100 |
| antibodies) | from | from | ID NO : | antibody | Linker | connector | stabilized |
| scFab- | <IGF1R> | <EGFR> | 8, 10, | C-terminus | (G₄S)₆GG | (G₄S)₂ | NO |
| XGFR1_2720 | | | 23, 25 | H chain | | | |
| scFab- | <IGF1R> | <EGFR> | 8, 10, | C-terminus | (G₄S)₆GG | (G₄S)₂ | YES |
| XGFR1_2721 | | | 23, 25 | H chain | | | |
| scFab- | <IGF1R> | <EGFR> | 8, 10, | C-terminus | (G₄S)₆GG | (G₄S)₂ | NO |
| XGFR1_4720 | | | 23, 25 | L chain | | | |
| scFab- | <IGF1R> | <EGFR> | 8, 10, | C-terminus | (G₄S)₆GG | (G₄S)₂ | YES |
| XGFR1_4721 | | | 23, 25 | L chain | | | |
| scFab- | <EGFR> | <IGF1R> | 8, 10, | C-terminus | (G₄S)₆GG | (G₄S)₂ | NO |
| XGFR2_2720 | | | 23, 25 | H chain | | | |
| scFab- | <EGFR> | <IGF1R> | 8, 10, | C-terminus | (G₄S)₆GG | (G₄S)₂ | YES |
| XGFR2_2721 | | | 23, 25 | H chain | | | |
| scFab- | <EGFR> | <IGF1R> | 8, 10, | C-terminus | (G₄S)₆GG | (G₄S)₂ | NO |
| XGFR2_4720 | | | 23, 25 | L chain | | | |
| scFab- | <EGFR> | <IGF1R> | 8, 10, | C-terminus | (G₄S)₆GG | (G₄S)₂ | YES |
| XGFR2_4721 | | | 23, 25 | L chain | | | |

Examples 9 to 13 refer to the tetravalent scFab-XGFR1 and scFab-XGFR2 molecules with single chain Fab attachments)

### Materials & General Methods

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to EU numbering (Edelman, G. M., et al., Proc. Natl. Acad. Sci. USA 63 (1969) 78-85; Kabat, E. A., et al., Sequences of Proteins of Immunological Interest, 5th ed., Public Health Service, National Institutes of Health, Bethesda, Md., (1991).

### Recombinant DNA Techniques

Standard methods were used to manipulate DNA as described in Sambrook, J., et al., Molecular cloning: A laboratory manual; Cold Spring Harbor Laboratory Press, Cold Spring Harbor, N.Y., 1989. The molecular biological reagents were used according to the manufacturer's instructions.

### Gene Synthesis

Desired gene segments were prepared from oligonucleotides made by chemical synthesis. The 600-1800 bp long gene segments, which are flanked by singular restriction endonuclease cleavage sites, were assembled by annealing and ligation of oligonucleotides including PCR amplification and subsequently cloned via the indicated restriction sites e.g. BamHI/BstEII, BamHI/BsiWI, BstEII/NotI or BsiWI/NotI into a pcDNA 3.1/Zeo(+) (Invitrogen) based on a pUC cloning vector. The DNA sequences of the subcloned gene fragments were confirmed by DNA sequencing. Gene synthesis fragments were ordered according to given specifications at Geneart (Regensburg, Germany).

### DNA Sequence Determination

DNA sequences were determined by double strand sequencing performed at Sequiserve GmbH (Vaterstetten, Germany).

### DNA and Protein Sequence Analysis and Sequence Data Management

The GCG's (Genetics Computer Group, Madison, Wis.) software package version 10.2 and Invitrogens Vector NT1 Advance suite version 9.1 was used for sequence creation, mapping, analysis, annotation and illustration.

### Cell Culture Techniques

Standard cell culture techniques were used as described in Current Protocols in Cell Biology (2000), Bonifacino, J., S., Dasso, M., Harford, J., B., Lippincott-Schwartz, J., and Yamada, K., M. (eds.), John Wiley & Sons, Inc.

### Transient Expression of Immunoglobulin Variants in HEK293F Cells

The bispecific antibodies were expressed by transient transfection of human embryonic kidney 293-F cells using the FreeStyle™ 293 Expression System according to the manufacturer's instruction (Invitrogen, USA). Briefly, suspension FreeStyle™ 293-F cells were cultivated in FreeStyle™ 293 Expression medium at 37° C./8% CO₂ and the cells were seeded in fresh medium at a density of 1-2×10⁶ viable cells/ml on the day of transfection. The DNA-293fectin™ complexes were prepared in Opti-MEM® I medium (Invitrogen, USA) using 333 μl of 293fectin™ (Invitrogen, Germany) and 250 μg of heavy and light chain plasmid DNA in a 1:1 molar ratio for a 250 ml final transfection volume. Bispecific antibody containing cell culture supernatants were clarified 7 days after transfection by centrifugation at 14000 g for 30 minutes and filtration through a sterile filter (0.22 μm). Supernatants were stored at −20° C. until purification.

### Protein Determination

The protein concentration of purified antibodies and derivatives was determined by determining the optical density (OD) at 280 nm with the OD at 320 nm as the background correction, using the molar extinction coefficient calculated on the basis of the amino acid sequence according to Pace, C. N., et. al., Protein Science, 4 (1995) 2411-1423.

### Antibody Concentration Determination in Supernatants

The concentration of antibodies and derivatives in cell culture supernatants was measured by affinity HPLC chromatography. Briefly, cell culture supernatants containing antibodies and derivatives that bind to Protein A were applied to an Applied Biosystems Poros A/20 column in 200 mM KH2PO4, 100 mM sodium citrate, pH 7.4 and eluted from the matrix with 200 mM NaCl, 100 mM citric acid, pH 2.5 on an UltiMate 3000 HPLC system (Dionex). The eluted protein was quantified by UV absorbance and integration of peak areas. A purified standard IgG1 antibody served as a standard.

### Protein Purification

The secreted antibodies were purified from the supernatant in two steps by affinity chromatography using Protein A-Sepharose™ (GE Healthcare, Sweden) and Superdex200 size exclusion chromatography. Briefly, the bispecific and trispecific antibody containing clarified culture supernatants were applied on a HiTrap ProteinA HP (5 ml) column equilibrated with PBS buffer (10 mM Na₂HPO₄, 1 mM KH₂PO₄, 137 mM NaCl and 2.7 mM KCl, pH 7.4). Unbound proteins were washed out with equilibration buffer. The bispecific antibodies were eluted with 0.1 M citrate buffer, pH 2.8, and the protein containing fractions were neutralized with 0.1 ml 1 M Tris, pH 8.5. Then, the eluted protein fractions were pooled, concentrated with an Amicon Ultra centrifugal filter device (MWCO: 30 K, Millipore) to a volume of 3 ml and loaded on a Superdex200 HiLoad 120 ml 16/60 gel filtration column (GE Healthcare, Sweden) equilibrated with 20 mM Histidin, 140 mM NaCl, pH 6.0. Monomeric antibody fractions were pooled, snap-frozen and stored at −80° C. Parts of the samples were provided for subsequent protein analytics and characterization.

### Analysis of Purified Proteins

The protein concentration of purified protein samples was determined by measuring the optical density (OD) at 280 nm, using the molar extinction coefficient calculated on the basis of the amino acid sequence. The purity of the bispecific antibodies were analyzed by SDS-PAGE in the presence and absence of a reducing agent (5 mM 1,4-dithiotreitol) and staining with Coomassie brilliant blue. The NuPAGE® Pre-Cast gel system (Invitrogen, USA) was used according to the manufacturer's instruction (4-20% Tris-Glycine gels). The aggregate content of bispecific antibody samples was analyzed by high-performance SEC on an UltiMate 3000 HPLC system (Dionex) using a Superdex 200 analytical size-exclusion column (GE Healthcare, Sweden) in 200 mM KH₂PO₄, 250 mM KCl, pH 7.0 running buffer at 25° C. 25 μg protein were injected on the column at a flow rate of 0.5 ml/min and eluted isocratic over 50 minutes. For stability analysis, concentrations of 0.1 mg/ml, 1 mg/ml and 3 mg/ml of purified proteins were prepared and incubated at 4° C., 37° C. for 7 days and then evaluated by high-performance SEC. The integrity of the amino acid backbone of reduced bispecific antibody light and heavy chains was verified by NanoElectrospray Q-TOF mass spectrometry after removal of N-glycans by enzymatic treatment with Peptide-N-Glycosidase F (Roche Molecular Biochemicals).

### Example 1

### Expression & Purification of Bispecific <EGFR-IGF1R> Antibody XGFR1 Molecules

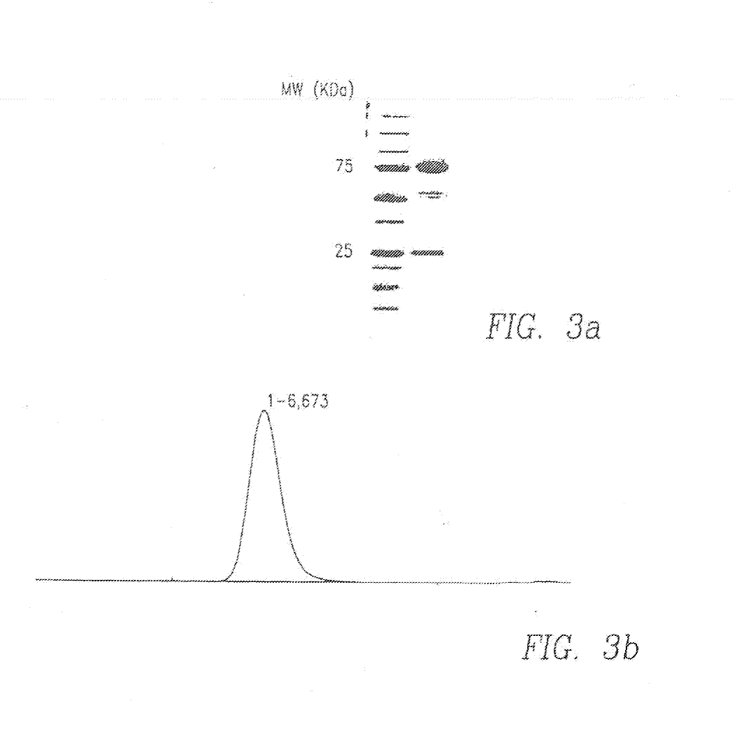

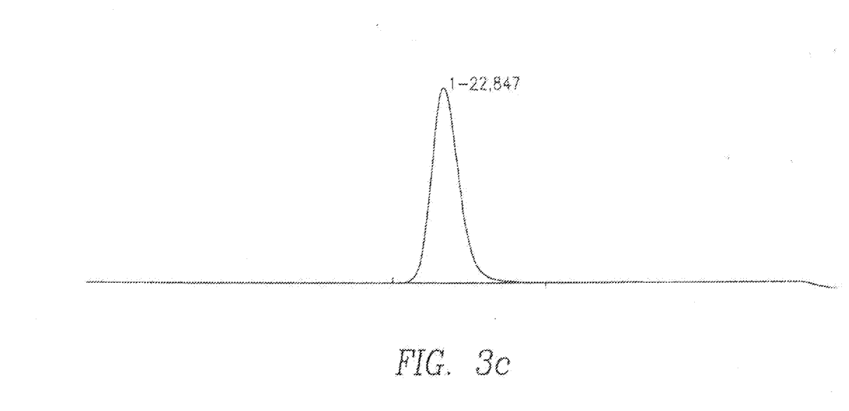

Light and heavy chains of the corresponding bispecific antibodies were constructed in expression vectors carrying pro- and eukaryotic selection markers. These plasmids were amplified in E. coli, purified, and subsequently transfected for transient expression of recombinant proteins in HEK293F cells (utilizing Invitrogen's freesyle system). After 7 days, HEK 293 cell supernatants were harvested and purified by protein A and size exclusion chromatography. Homogeneity of all bispecific antibody constructs was confirmed by SDS-PAGE under non reducing and reducing conditions. Under reducing conditions (FIG. 2a), polypeptide chains carrying C- and N-terminal scFv fusions showed upon SDS-PAGE apparent molecular sizes analogous to the calculated molecular weights. Expression levels of all constructs were analysed by Protein A HPLC and were similar to expression yields of ‘standard’ IgGs, or in some cases somewhat lower. Average protein yields were between 1 and 36 mg of purified protein per liter of cell-culture supernatant in such non-optimized transient expression experiments (FIG. 3). Non-disulfide stabilized constructs with C-terminal fused scFvs at the light (XGFR-4320) or heavy chain (XGFR-2320) showed higher amounts of recovered protein of desired size after protein A purification than N-terminal attached scFvs (XGFR1-3320 and XGFR1-5320).

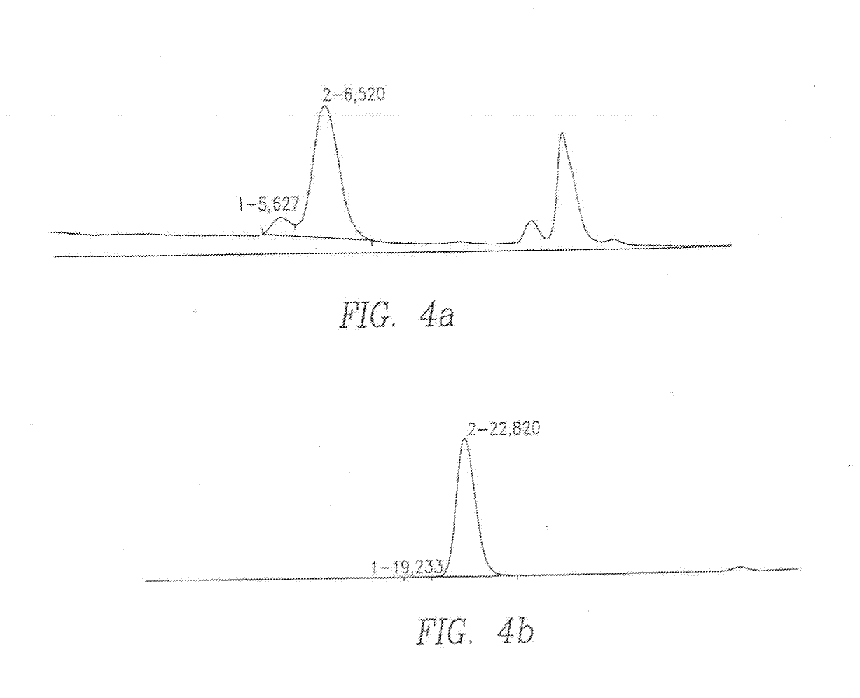

HP-Size exclusion chromatography analysis of the purified proteins showed (compared to ‘normal’ IgGs) some tendency to aggregate for molecules that contained scFvs that were not stabilized by interchain disulfides between VH and VL. To address the problems with aggregation of such bispecific antibodies, disulfide-stabilization of the scFv moieties was applied. For that we introduced single cysteine replacements within VH and VL of the scFv at defined positions (positions VH44/VL100 according to the Kabat numbering scheme). These mutations enable the formation of stable interchain disulfides between VH and VL, which in turn stabilize the resulting disulfide-stabilized scFv module. Introduction of the VH44/VL100 disulfides in scFvs at the N- and C-terminus of the Fv lead to an improvement in protein expression levels for all constructs (see FIG. 4).

The bispecific antibodies were expressed by transient transfection of human embryonic kidney 293-F cells using the FreeStyle™ 293 Expression System according to the manufacturer's instruction (Invitrogen, USA). Briefly, suspension FreeStyle™ 293-F cells were cultivated in FreeStyle™ 293 Expression medium at 37° C./8% CO₂ and the cells were seeded in fresh medium at a density of 1-2×10⁶ viable cells/ml on the day of transfection. The DNA-293fectin™ complexes were prepared in Opti-MEM® I medium (Invitrogen, USA) using 333 μl of 293fectin™ (Invitrogen, Germany) and 250 μg of heavy and light chain plasmid DNA in a 1:1 molar ratio for a 250 ml final transfection volume. Bispecific antibody containing cell culture supernatants were clarified 7 days after transfection by centrifugation at 14000 g for 30 minutes and filtration through a sterile filter (0.22 μm). Supernatants were stored at −20° C. until purification.

The secreted antibodies were purified from the supernatant in two steps by affinity chromatography using Protein A-Sepharose™ (GE Healthcare, Sweden) and Superdex200 size exclusion chromatography. Briefly, the bispecific and trispecific antibody containing clarified culture supernatants were applied on a HiTrap ProteinA HP (5 ml) column equilibrated with PBS buffer (10 mM Na₂HPO₄, 1 mM KH₂PO₄, 137 mM NaCl and 2.7 mM KCl, pH 7.4). Unbound proteins were washed out with equilibration buffer. The bispecific antibodies were eluted with 0.1 M citrate buffer, pH 2.8, and the protein containing fractions were neutralized with 0.1 ml 1 M Tris, pH 8.5. Then, the eluted protein fractions were pooled, concentrated with an Amicon Ultra centrifugal filter device (MWCO: 30 K, Millipore) to a volume of 3 ml and loaded on a Superdex200 HiLoad 120 ml 16/60 gel filtration column (GE Healthcare, Sweden) equilibrated with 20 mM Histidin, 140 mM NaCl, pH 6.0. Monomeric antibody fractions were pooled, snap-frozen and stored at −80° C. Part of the samples were provided for subsequent protein analytics and characterization.

XGFR1-2320 had a final yield after purification of 0.27 mg whereas XGFR1-2321 had a final yield of 13.8 mg.

Exemplary SDS-PAGE and HP-Size Exclusion Chromatography (SEC) purification and analysis of bispecific antibodies is shown in FIGS. 3 and 4.

### Example 2

### Stability of Bispecific <EGFR-IGF1R> Antibody XGFR1 Molecules In Vitro

### HP-Size Exclusion Chromatography Analysis was Performed

The aggregate content of bispecific antibody samples was analyzed by high-performance SEC on an UltiMate 3000 HPLC system (Dionex) using a Superdex 200 analytical size-exclusion column (GE Healthcare, Sweden) in 200 mM KH₂PO₄, 250 mM KCl, pH 7.0 running buffer at 25° C. 25 μg protein were injected on the column at a flow rate of 0.5 ml/min and eluted isocratic over 50 minutes. For stability analysis, concentrations of 0.1 mg/ml, 1 mg/ml and 5 mg/ml of purified proteins were prepared and incubated at 4° C., 37° C. and 40° C. for 7 or 28 days and then evaluated by high-performance SEC. The integrity of the amino acid backbone of reduced bispecific antibody light and heavy chains was verified by NanoElectrospray Q-TOF mass spectrometry after removal of N-glycans by enzymatic treatment with Peptide-N-Glycosidase F (Roche Molecular Biochemicals).

HP-Size exclusion chromatography analysis of the purified proteins under different conditions (varying concentration and time) showed-compared to normal IgGs-0 a greatly increased tendency to aggregate for molecules that contained scFvs.

For this work, we define desired ‘monomeric’ molecules to be composed of 2 heterodimers of heavy and light chains-with scFvs connected to either of both.

In contrast to the strong aggregation tendency of entities that contained unmodified scFvs, HP Size exclusion analysis of the VH44/VL100 disulfide stabilized constructs showed much less tendency to aggregate.

Exemplary HP-Size Exclusion Chromatography (SEC) purification and analysis of bispecific antibodies is shown in FIG. 4.

### Example 3

### Simultaneous Binding of Bispecific <EGFR-IGF1R> Antibody XGFR1 molecules to the RTKs EGFR and IGF1R

The binding of the scFv modules and of the Fvs retained in the IgG-module of the different bispecific antibody formats were compared to the binding of the ‘wildtype’ IgGs from which the binding modules and bispecific antibodies were derived. These analyses were carried out by applying Surface Plasmon Resonance (Biacore), as well as a cell-ELISA.

The binding properties bispecific anti-IGF-1R/anti-EGFR antibodies were analyzed by surface plasmon resonance (SPR) technology using a Biacore T100 instrument (Biacore AB, Uppsla). This system is well established for the study of molecule interactions. It allows a continuous real-time monitoring of ligand/analyte bindings and thus the determination of association rate constants (ka), dissociation rate constants (kd), and equilibrium constants (KD) in various assay settings. SPR-technology is based on the measurement of the refractive index close to the surface of a gold coated biosensor chip. Changes in the refractive index indicate mass changes on the surface caused by the interaction of immobilized ligand with analyte injected in solution. If molecules bind to immobilized ligand on the surface the mass increases, in case of dissociation the mass decreases.

Capturing anti-human IgG antibody was immobilized on the surface of a CM5 biosensorchip using amine-coupling chemistry. Flow cells were activated with a 1:1 mixture of 0.1 M N-hydroxysuccinimide and 0.1 M 3-(N,N-dimethylamino)propyl-N-ethylcarbodiimide at a flow rate of 5 μl/min Anti-human IgG antibody was injected in sodium acetate, pH 5.0 at 10 μg/ml, which resulted in a surface density of approximately 12000 RU. A reference control flow cell was treated in the same way but with vehicle buffers only instead of the capturing antibody. Surfaces were blocked with an injection of 1 M ethanolamine/HCl pH 8.5. The bispecific antibodies were diluted in HBS-P and injected at a flow rate of 5 μl/min. The contact time (association phase) was 1 min for the antibodies at a concentration between 1 and 5 nM for the EGFR-ECD binding and 20 nM for the IGF-1R interaction. EGFR-ECD was injected at increasing concentrations of 3.125, 6.25, 12.5, 25, 50 and 100 nM, IGF-1R at concentrations of 0.21, 0.62, 1.85, 5.6, 16.7 and 50 nM. The contact time (association phase) was 3 min, the dissociation time (washing with running buffer) 5 min for both molecules at a flowrate of 30 μl/min. All interactions were performed at 25° C. (standard temperature). The regeneration solution of 3 M Magnesium chloride was injected for 60 s at 5 μl/min flow to remove any non-covalently bound protein after each binding cycle. Signals were detected at a rate of one signal per second. Samples were injected at increasing concentrations.

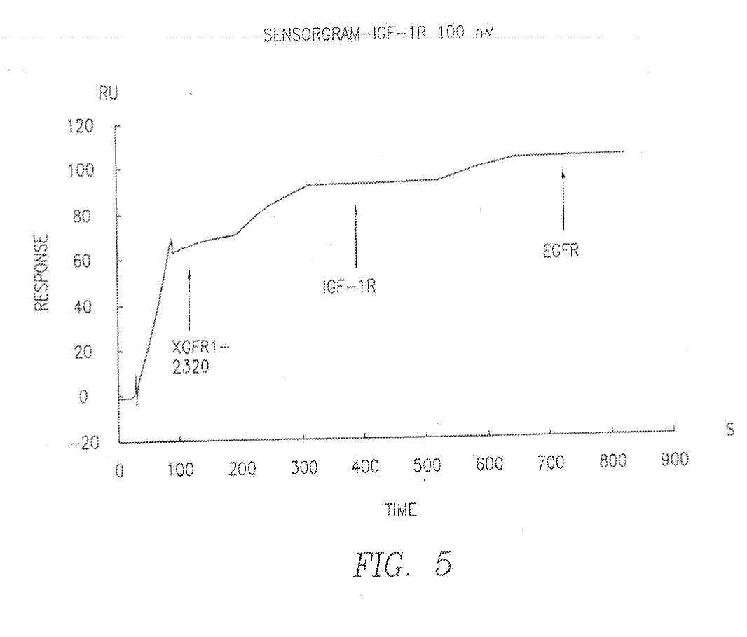

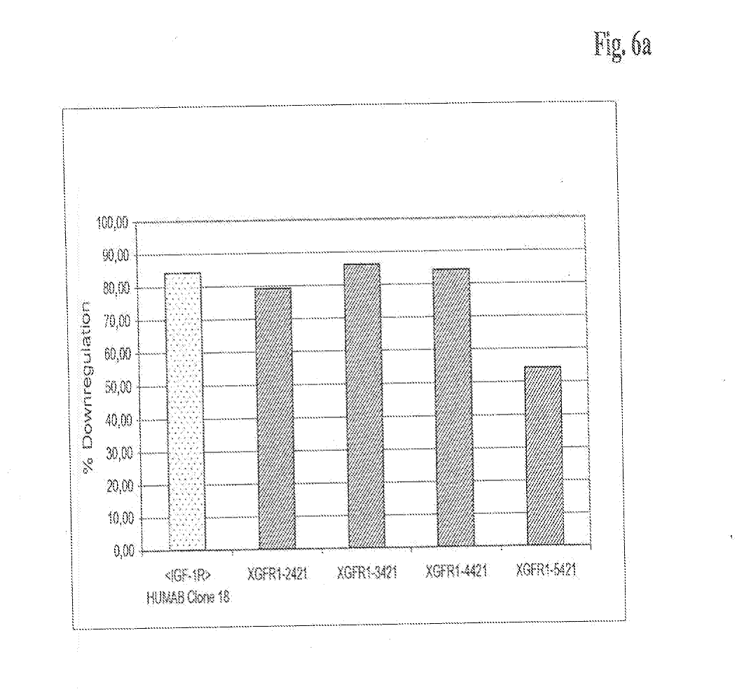

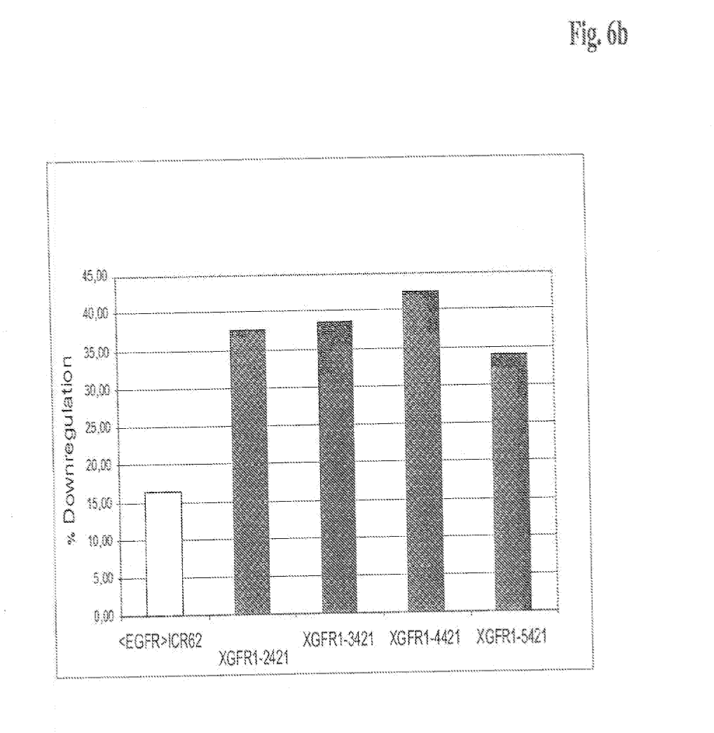

Exemplary simultaneous binding of an bispecific antibody to EGFR and IGF1R is shown in FIG. 5.

TABLE 4
Affinities (KD) of bispecific antibodies
(XGFR -nomenclature) to EGFR and IGF-1R

| | KD value | KD value |
|---|---|---|
| Molecule | (Affinity to EGFR) | (Affinity to IGF-1R) |
| XGFR1-2321 | 4 nM | n.d |
| XGFR1-2421 | 6 nM | 6 nM |
| XGFR1-3321 | 6 nM | 4 nM |
| XGFR1-3421 | 3 nM | 2 nM |
| XGFR1-4321 | 5 nM | n.d. |
| XGFR1-4421 | 3 nM | 5 nM |
| XGFR1-5321 | 4 nM | n.d. |
| XGFR1-5421 | 3 nM | 2 nM |
| <EGFR> ICR62 | 3 nM | n.d |
| <IGF-1R> | n.d. | 5 nM |
| HUMAB-Clone 18 | | |

### Example 4

### Downregulation of EGFR- as Well as IGF1R-by Bispecific <EGFR-IGF1R> Antibody XGFR1 Molecules

The human anti-IGF-1R antibodies <IGF-1R> HUMAB Clone 18 (DSM ACC 2587) inhibits IGFR1-signaling and the humanized rat anti-EGFR antibody <EGFR>ICR62 inhibits the signaling by EGFR. To evaluate the potential inhibitory activity of the different XGFR1 variants, the degree of downregulation of the receptor from both was analyzed.

In order to detect effects of the antibody of the invention on the amount of IGF-I receptor (IGF-IR) in tumor cells, time-course experiments and subsequent ELISA analysis with IGF-IR and EGFR specific antibodies were performed.

Human tumor cells (A549, 2×10⁵ cells/ml) in RPMI-VM medium (PAA, Cat. No. E15-039) supplemented with 1% PenStrep in one 6 well plate and was innoculated with 4 ml cells in the respective medium for each experiment and cultivated for 24 hours at 37° C. and 5% CO₂.

The medium was carefully removed and replaced by 2 ml 0.01 mg/ml XGFR antibodies diluted in RPMI-VM medium. In three control wells, medium was replaced by either medium without antibody, medium with a control antibodies (<IGF-1R> HUMAB Clone 18 and <EGFR>ICR62 final concentration 0.01 mg/ml) and a well containing only buffer. Cells were incubated at 37° C. and 5% CO₂ and individual plates were taken out for further processing after 24 hours.

The medium was carefully removed by aspiration and the cell were washed with 1 ml PBS. 300 μl/well of cold MES-lysis buffer was added (MES, 10 mM Na₃VO₄, and Complete® protease inhibitor). The cells were detached using a cell scraper (Corning, Cat. No. 3010) and the well contents transferred to Eppendorf reaction tubes. Cell fragments were removed by centrifugation for 10 minutes at 13000 rpm and 4° C.

### For EGFR Detection

The 96 well streptavidin microtitreplates (MTP) were prepared according to the protocol (DuoSet ELISA for Human EGFR, RnD systems Cat. No. DY231). The Human EGFR goat antibody 144 μg/ml in PBS was diluted 1:180 in PBS and 100 μl/well was added to the MTP. The MTP was incubated overnight at 4° C. with agitation. The plates were washed 3 times with PBS supplemented with 0.1% Tween®20 and blocked with 300 μl/well of PBS, 3% BSA and 0.1% Tween®20 solution for 1 hour (h) at room temperature (RT) with agitation. The plates were washed 3 times with PBS supplemented with 0.1% Tween®20.

The amount of protein in the cell lysates was determined using the BCA Protein Assay kit (Pierce), the cell lysates were then adjusted to a protein concentration of 0.1 mg/ml with MES-lysis buffer supplemented with 100 mM Na₃VO₄ 1:100 and Complete® protease inhibitor 1:20 and 100 μl per well of the lysate was added to the pre-prepared MTP.

A second cell lysate concentration was used at 0.05 mg/ml the lysate was dilute 1:2 and 100 μl was added per well to the pre-prepared MTP. The MTP were incubated for a further 2 hour at RT with agitation and then washed 3 times with PBS with 0.1% Tween®20 solution.

The detection antibody for EGFR was human EGFR goat biotinylated antibody at a concentration of 36 μg/ml diluted 1:180 in PBS, 3% BSA and 0.2% Tween®20, 100 μl per well was added and incubated at RT for 2 hours with agitation. The MTP was then washed three times with 200 μl per well of PBS with 0.1% Tween®20 solution. The secondary antibody was then added Streptavidin-HRP 1:200 in PBS, 3% BSA and 0.2% Tween®20 100 μl per well and incubated with agitation for 20 minutes at RT. The plate was then washed six times with PBS with 0.1% Tween®20 solution. 100 μl per well of 3,3′-5,5′-Tetramethylbenzidin (Roche, BM-Blue ID-No.: 11484581) was added and incubated for 20 minutes at RT with agitation. The colour reaction was stopped by adding 25 μl per well of 1M H₂SO₄ and incubating for a further 5 minutes at RT. The absorbance was measured at 450 nm.

### For IGF-1R Detection

The streptavidin-MTP (Roche ID. No.: 11965891001) was prepared by adding 100 μl per well of the antibody AK1a-Biotinylated (Genmab, Denmark) which was diluted 1:200 in PBS, 3% BSA and 0.2% Tween®20. The streptavidin-MTP was incubated for 1 hour at RT with agitation and then washed three times with 200 μl per well of PBS with 0.1% Tween®20 solution.

The amount of protein in the cell lysates was determined using the BCA Protein Assay kit (Pierce), the cell lysates were then adjusted to a protein concentration of 0.04 mg/ml with 50 mM Tris pH 7.4, 100 mM Na₃VO₄ 1:100 and Complete® protease inhibitor 1:20 and 100 μl per well of the lysate was added to the pre-prepared streptavidin-MTP.

A second cell lysate concentration was used at 0.02 mg/ml the lysate was dilute and 100 μl was added per well to the pre-prepared streptavidin-MTP. The positive control containing the unstimulated cells was diluted to 1:4000 in lysis buffer supplemented with 50 mM Tris pH 7.4, 100 mM Na₃VO₄ 1:100 and Complete® protease inhibitor 1:20 and 100 μl per well of the lysate was added to the pre-prepared streptavidin-MTP. For the negative control 100 μl lysis buffer was added to the well in the streptavidin-MTP.

The MTP were incubated for a further 1 hour at RT with agitation and then washed 3 times with PBS with 0.1% Tween®20 solution.

The detection antibody for IGF-1R was human IGF-1Rβ rabbit antibody (Santa Cruz Biotechnology, Cat. No. sc-713) diluted 1:750 in PBS, 3% BSA and 0.2% Tween®20, 100 μl per well was added and incubated at RT for 1 hour with agitation. The MTP was then washed three times with 200 μl per well of PBS with 0.1% Tween®20 solution. The secondary antibody was then added rabbit IgG-POD (Cell signaling Cat. No. 7074) 1:4000 in PBS, 3% BSA and 0.2% Tween®20, 100 μl was added per well and incubated with agitation for 1 hour at RT. The plate was then washed six times with PBS with 0.1% Tween®20 solution. 100 μl per well of 3,3′-5,5′-Tetramethylbenzidin (Roche, BM-Blue ID-No.: 11484581) was added and incubated for 20 minutes at RT with agitation. The colour reaction was stopped by adding 25 μl per well of 1M H₂SO₄ and incubating for a further 5 minutes at RT. The absorbance was measured at 450 nm.

The results of the receptor downregulation detection by the bispecific antibodies XGFR compared to the parent monospecific antibodies <EGFR>ICR62 and <IGF-1R> HUMAB-Clone 18 in A549 cells is shown in FIG. 12. The bispecific antibodies XGFR downregulate both EGFR- as well as the IGF1R. Surprisingly the bispecific antibodies XGFR, showed an improved downregulation EGFR compared to the parent <EGFR>ICR62 antibody.

### Example 5

### Inhibition of EGFR- as Well as IGF1R-Signaling Pathways by Bispecific <EGFR-IGF1R> Antibody XGFR1 Molecules

The human anti-IGF-1R antibodies <IGF-1R> HUMAB Clone 18 (DSM ACC 2587) inhibits IGFR1-signaling and the humanized rat anti-EGFR antibody ICR62 inhibits the signaling by EGFR. To evaluate the potential inhibitory activity of the different XGFR1 variants, the degree of inhibition of signaling towards both pathways was analyzed.

Human tumor cells (H322M, 2×10⁵ cells/ml) in RPMI medium (PAA, Cat. No. E15-039) supplemented with 1% PenStrep in one 6 well plate and was innoculated with 4 ml cells in the respective medium for each experiment and cultivated for 24 hours at 37° C. and 5% CO₂.

The medium was carefully removed and replaced by 2 ml 0.01 mg/ml XGFR antibodies diluted in RPMI-VM medium. In three control wells, medium was replaced by either medium without antibody, medium with a control antibodies (<IGF-1R> HUMAB Clone 18 and <EGFR>ICR62 final concentration 0.01 mg/ml) and a well containing only buffer. Cells were incubated at 37° C. and 5% CO₂ and individual plates were taken out for further processing after 24 hours.

### For EGFR Phosphorylation Detection

The DuoSet® IC Human phospho-EGF R, RnD systems Cat. No. DYC1095-5 was used. The plates were prepared by diluting the Phospho EGF R capture antibody (Cat. No. 841402) to a concentration of 0.8 μg/ml. 100 μl per well of the MTP was added the plate was sealed and incubated overnight at RT.

The capture antibody was then aspirated and each well was washed with 400 μl wash buffer (0.05% Tween®20 in PBS pH 7.2-7.4 Cat No. WA126) five times after the final wash blot the plate on clean paper towels.

The plates were blocked by adding 300 μl of blocking buffer (1% BSA, 0.05% NaN₃ in PBS pH 7.2-7.4) and incubating at RT for 2 hours. The solution was then aspirated and each well was washed with 400 μl wash buffer (0.05% Tween®20 in PBS pH 7.2-7.4 Cat No. WA126) five times after the final wash the plates were blotted on clean paper towels.

The cells were rinsed in PBS and lysed using lysis buffer 9 (1% NP-40, 20 mM Tris pH8.0, 137 mM NaCl, 10% Glycerol, 2 mM EDTA, 1 mM activated sodium orthovanadate, 10 μg/ml Aprotinin and 10 μg/ml Leupeptin) at a cell density of 1×10⁷ cells/ml and incubated at 4° C. for 30 minutes with gentle agitation. The samples were then centrifuged at 14,000 g for 5 minutes. The samples were then transferred to a clean test tube.

The amount of protein in the cell lysates was determined using the BCA Protein Assay kit (Pierce), the cell lysates were then adjusted to a protein concentrations of 0.1 mg/ml and 0.05 mg/ml with IC Diluent 12 (1% NP-40, 20 mM Tris pH8.0, 137 mM NaCl, 10% Glycerol, 2 mM EDTA, 1 mM activated sodium orthovanadate). 100 μl per well of the lysate was added to the pre-prepared MTP the plate was sealed and incubated for 2 hours at RT.

Immediately before use the detection antibody was diluted to the working concentration specified on the vial in IC Diluent 14 (20 mM Tris, 137 mM NaCl, 0.05% Tween®20, 0.1% BSA, pH 7.2-7.4). 100 μl of the detection antibody was added per well, the plate was sealed and incubated at RT for 2 hours in the dark. The detection antibody was then aspirated and each well was washed with 400 μl wash buffer (0.05% Tween®20 in PBS pH 7.2-7.4 Cat No. WA126) five times after the final wash blot the plate on clean paper towels.

100 μl of substrate solution (Cat. No. DY999) was added per well and the plate was incubated in the dark for a further 20 minutes. The reaction was stopped by adding 50 μl stop solution 2N H2SO4 (Cat No. DY994) and mixing thoroughly.

The absorbance at 450 nm was measured.

### For IGF-1R Phosphorylation Detection

The streptavidin-MTP (Roche ID. No.: 11965891001) was prepared by adding 100 μl per well of the antibody AK1a-Biotinylated (Genmab, Denmark) which was diluted 1:200 in PBS, 3% BSA and 0.2% Tween®20. The streptavidin-MTP was incubated for 1 hour at RT with agitation and then washed three times with 200 μl per well of PBS with 0.1% Tween®20 solution.

The amount of protein in the cell lysates was determined using the BCA Protein Assay kit (Pierce), the cell lysates were then adjusted to a protein concentration of 1 μM with 50 mM Tris pH 7.4, 100 mM Na₃VO₄ 1:100 and Complete protease inhibitor 1:20 and 100 μl per well of the lysate was added to the pre-prepared streptavidin-MTP.

The positive control containing the unstimulated cells was diluted to 1:4000 in lysis buffer supplemented with 50 mM Tris pH 7.4, 100 mM Na₃VO₄ 1:100 and Complete® protease inhibitor 1:20 and 100 μl per well of the lysate was added to the pre-prepared streptavidin-MTP. For the negative control 100 μl lysis buffer was added to the well in the streptavidin-MTP.

The MTP were incubated for a further 1 hour at RT with agitation and then washed 3 times with PBS with 0.1% Tween®20 solution.

The detection antibody for IGF-1R was human IGF-1R (Tyr1135/1136)/Insulin receptor beta (Tyr1150/1151)(19H7) antibody (Cell signalling, Cat. No. 3024L) diluted 1:500 in PBS, 3% BSA and 0.2% Tween®20, 100 μl per well was added and incubated at RT for 1 hour with agitation. The MTP was then washed three times with 200 μl per well of PBS with 0.1% Tween®20 solution. The secondary antibody was then added rabbit IgG-POD (Cell signalling Cat. No. 7074) 1:4000 in PBS, 3% BSA and 0.2% Tween®20, 100 μl was added per well and incubated with agitation for 1 hour at RT. The plate was then washed six times with PBS with 0.1% Tween®20 solution. 100 μl per well of 3,3′-5,5′-Tetramethylbenzidin (Roche, BM-Blue ID-No.: 11484581) was added and incubated for 20 minutes at RT with agitation. The colour reaction was stopped by adding 25 μl per well of 1M H₂SO₄ and incubating for a further 5 minutes at RT. The absorbance was measured at 450 nm.

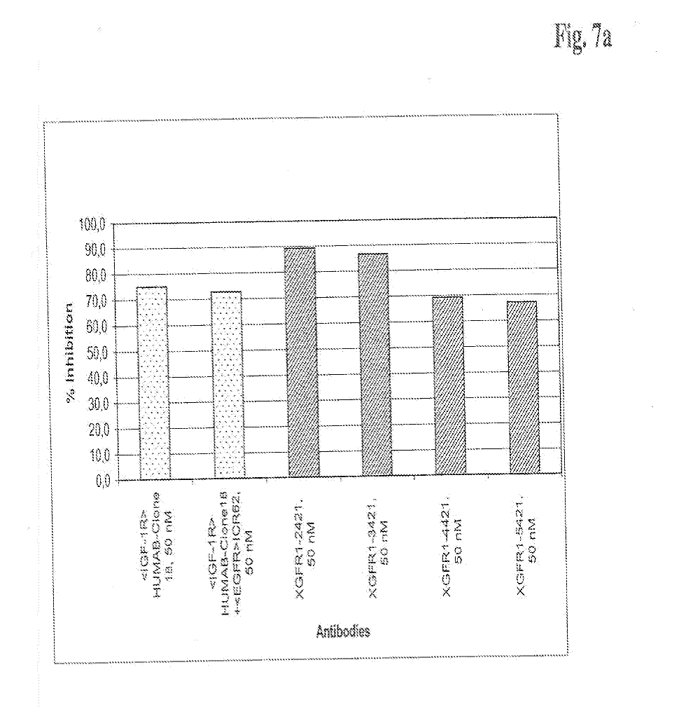

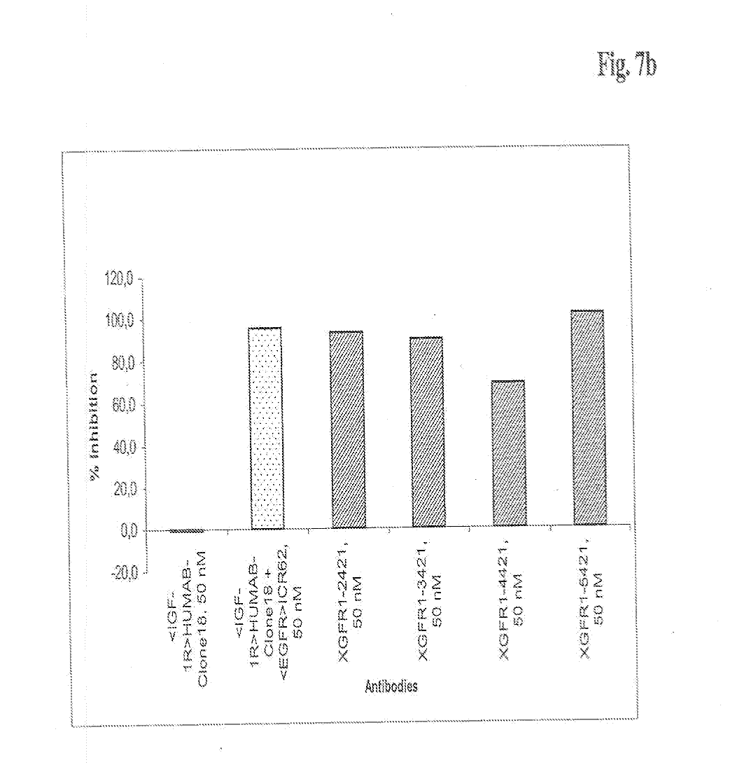

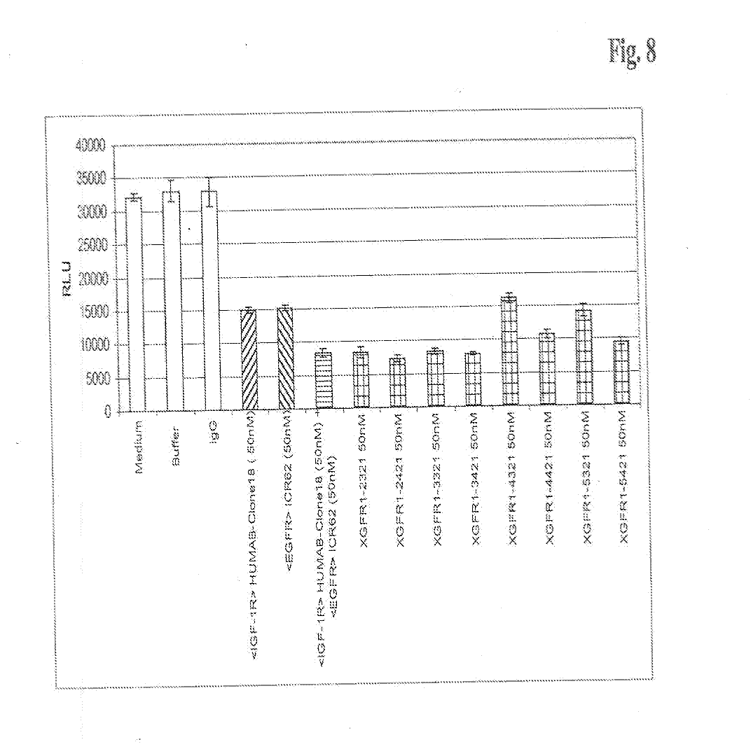

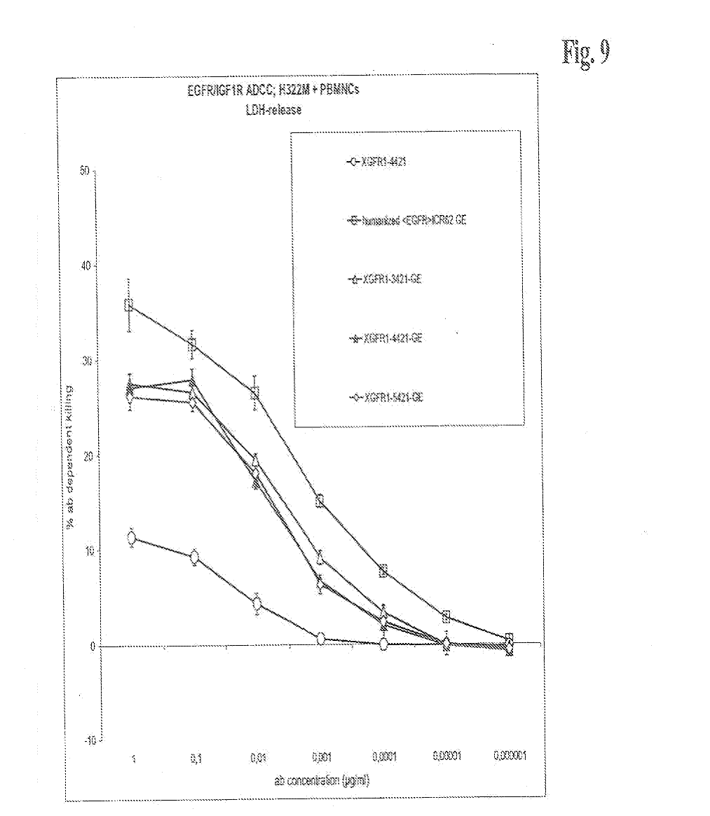

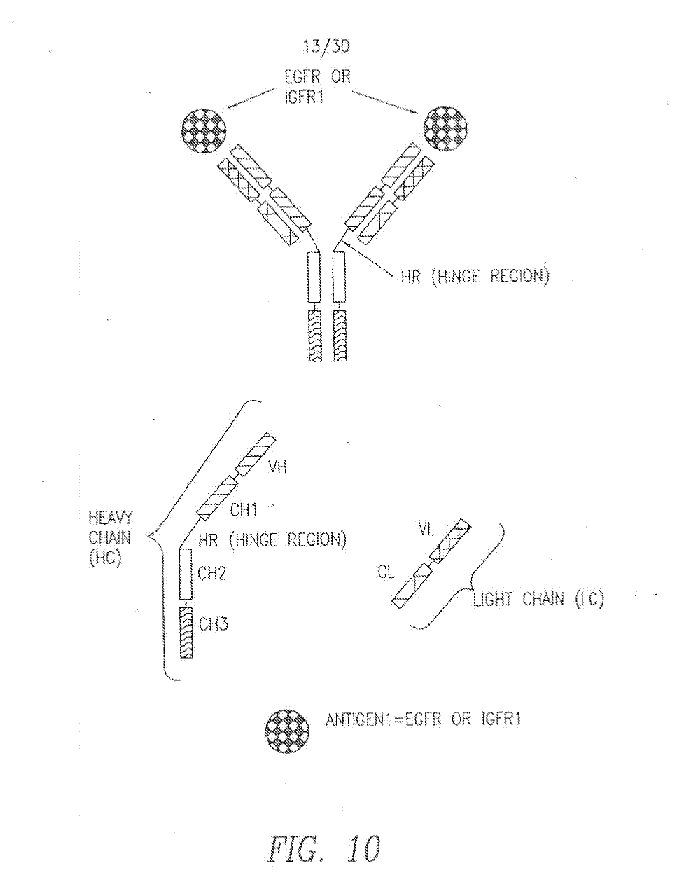

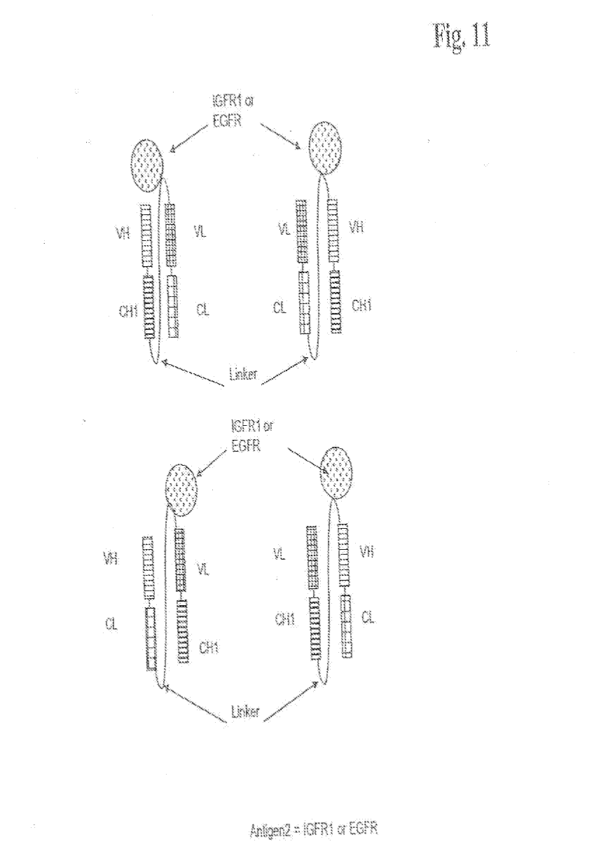

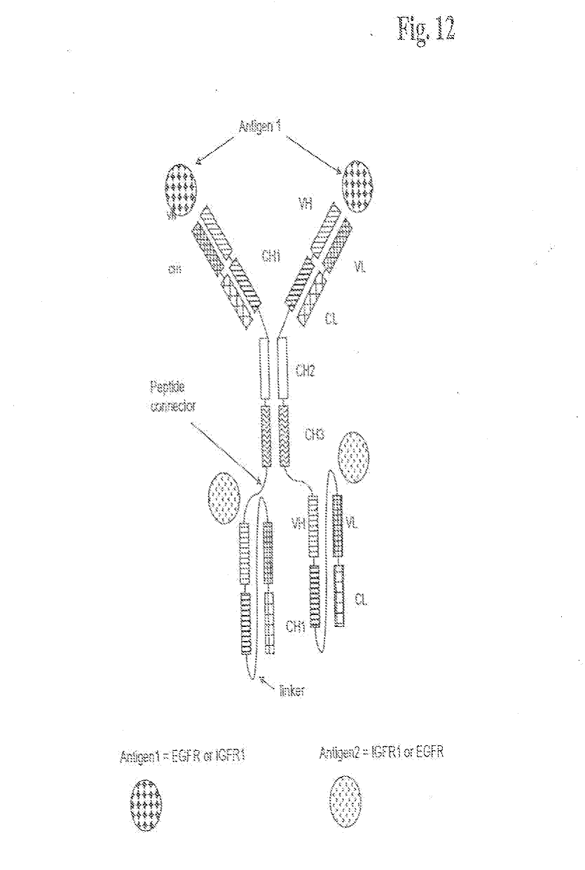

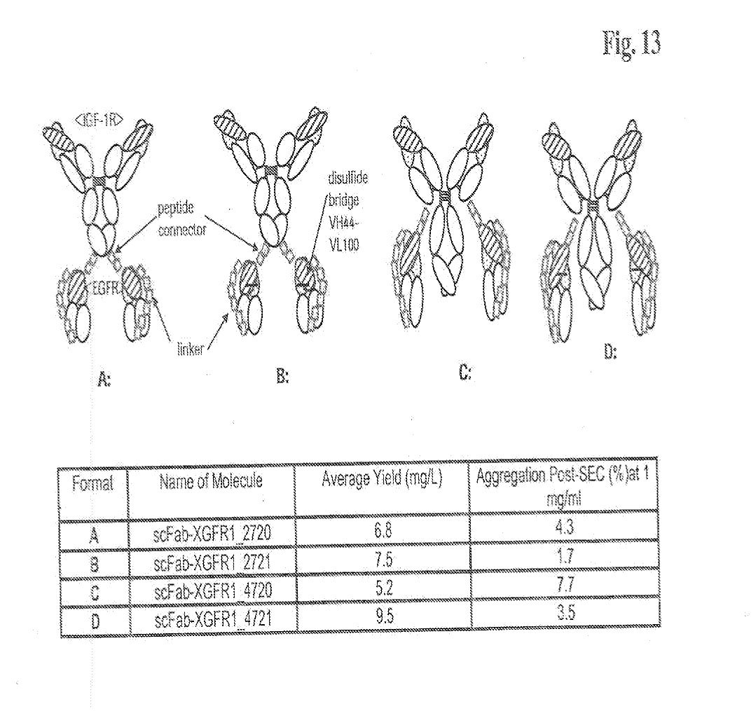

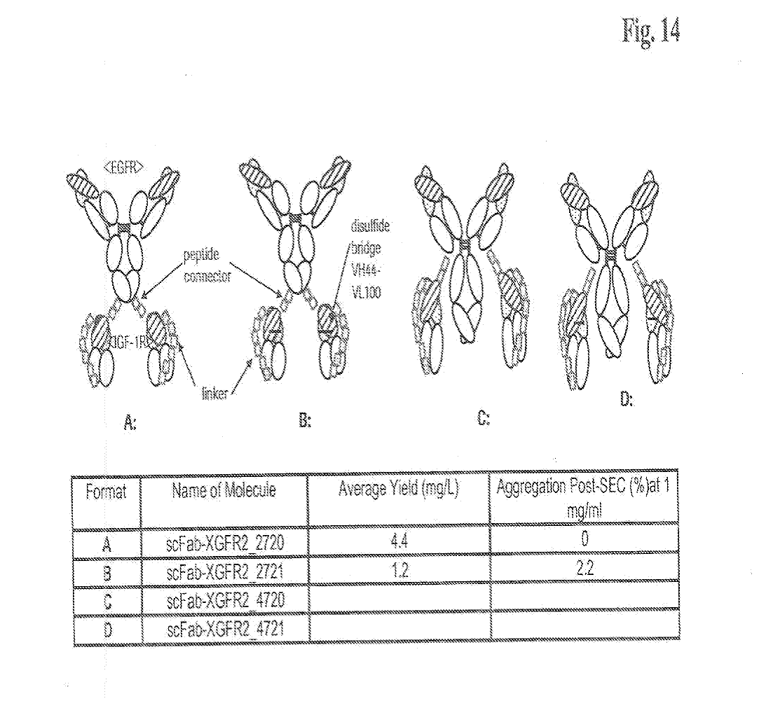

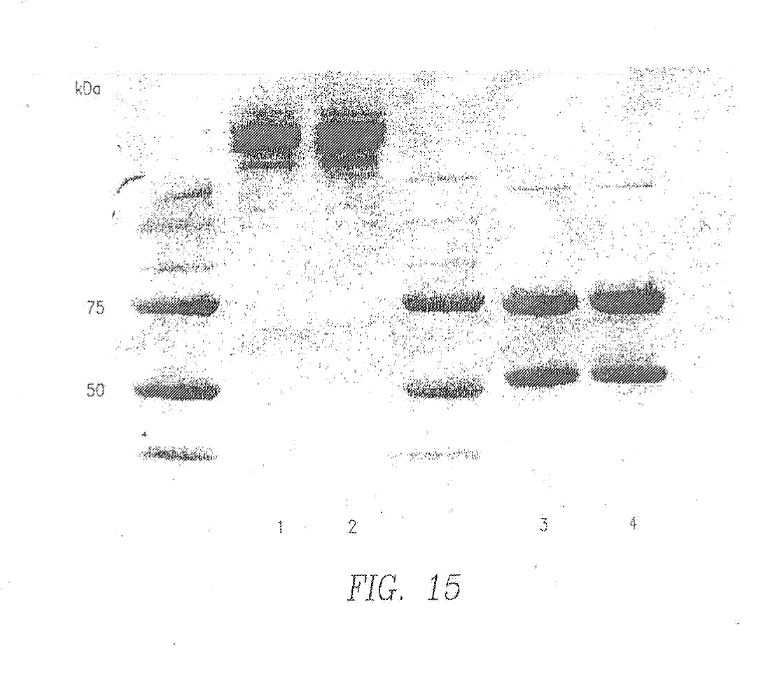

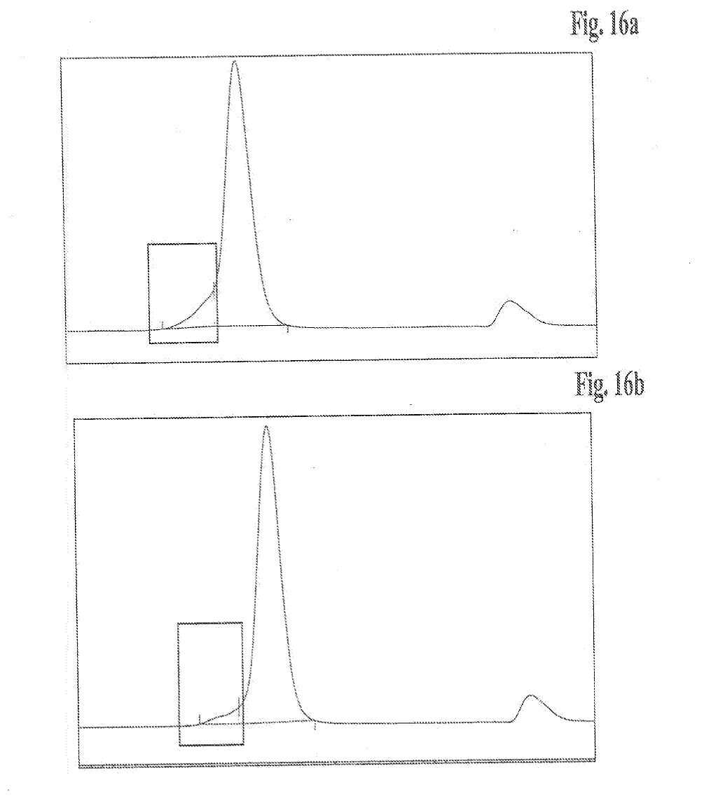

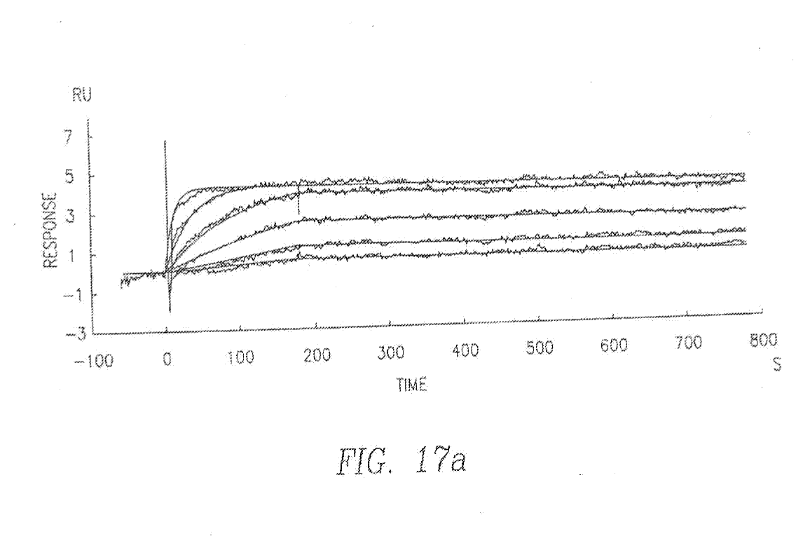

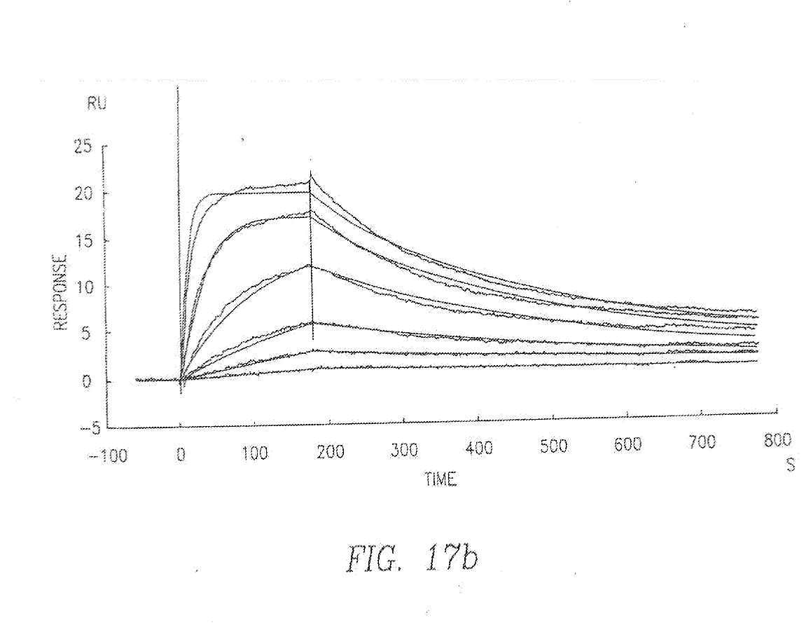

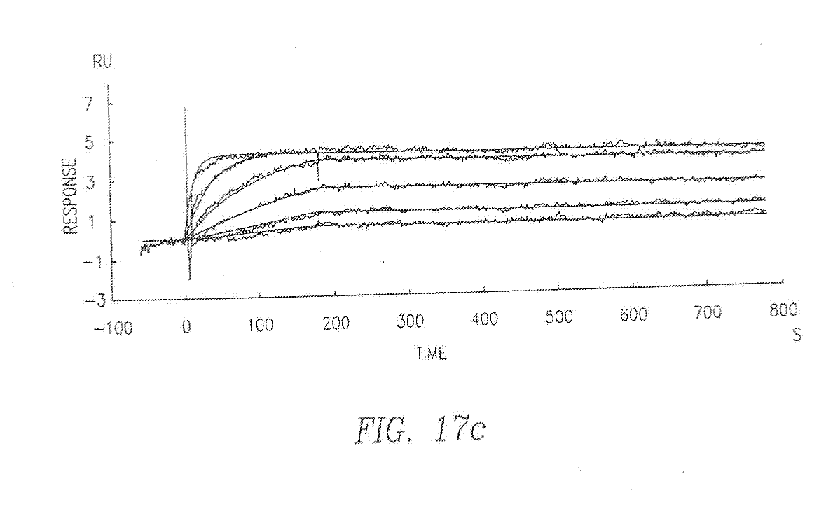

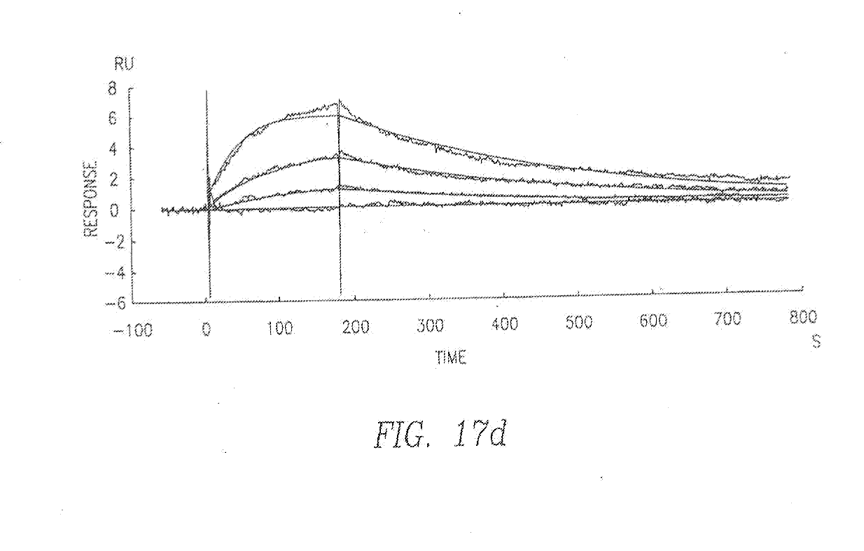

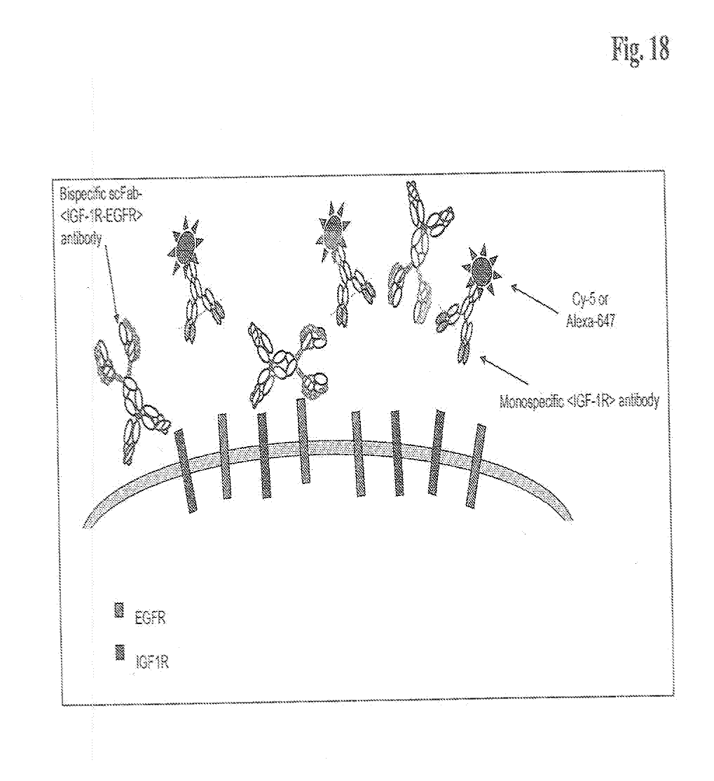

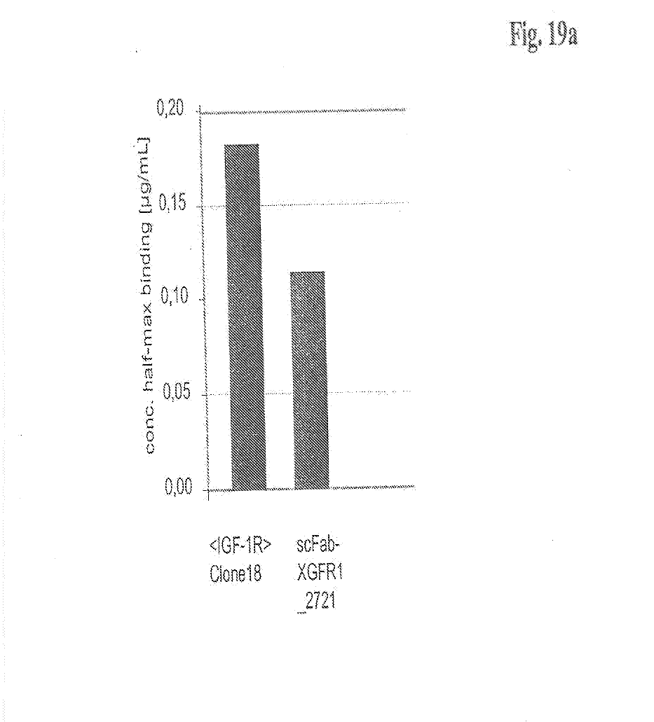

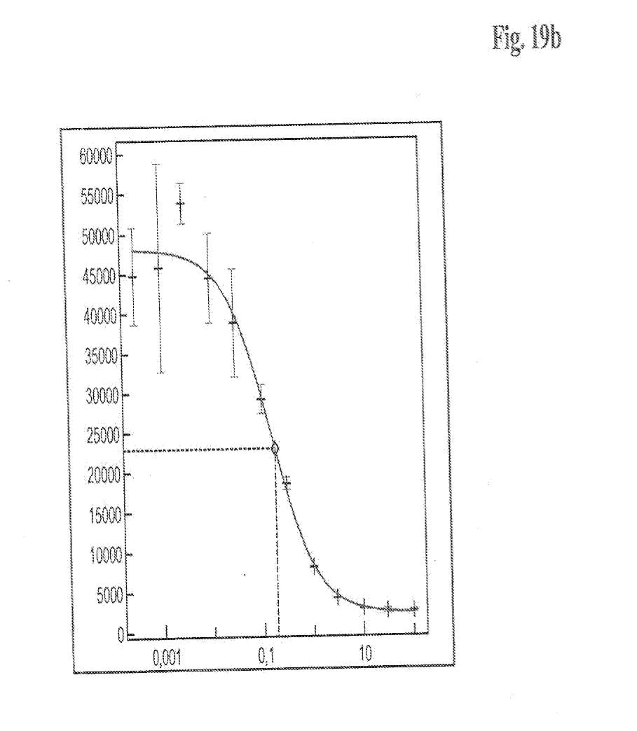

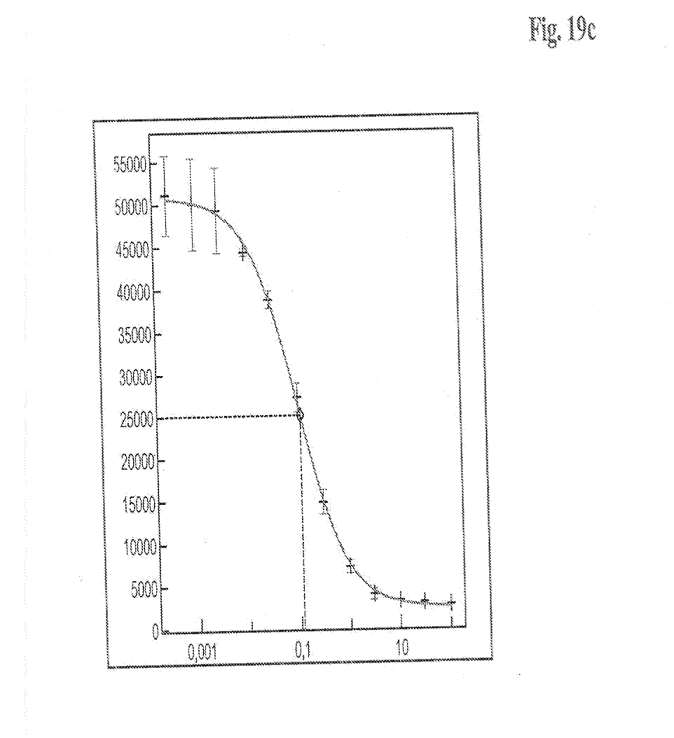

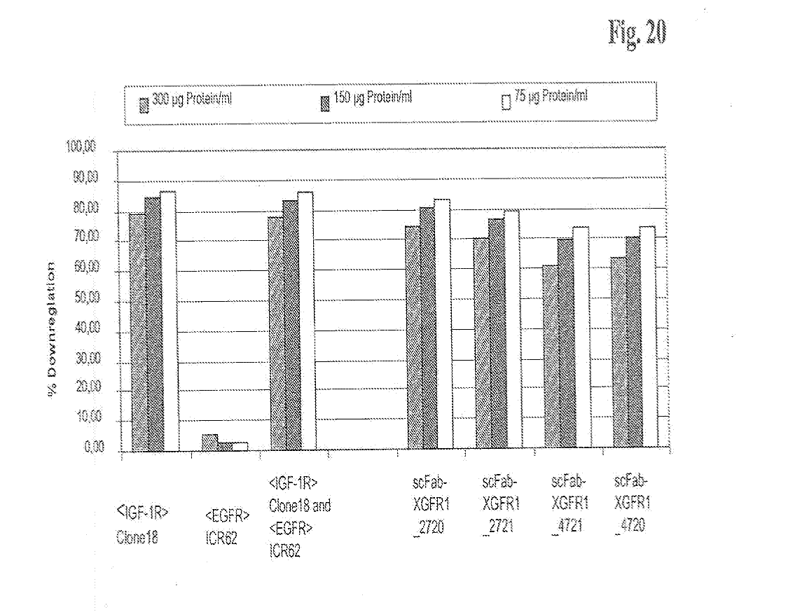

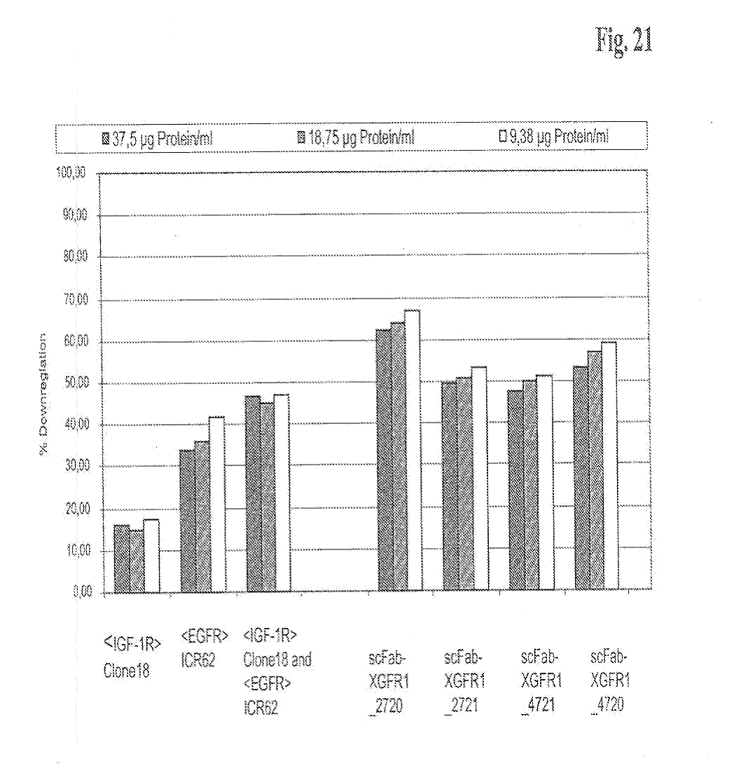

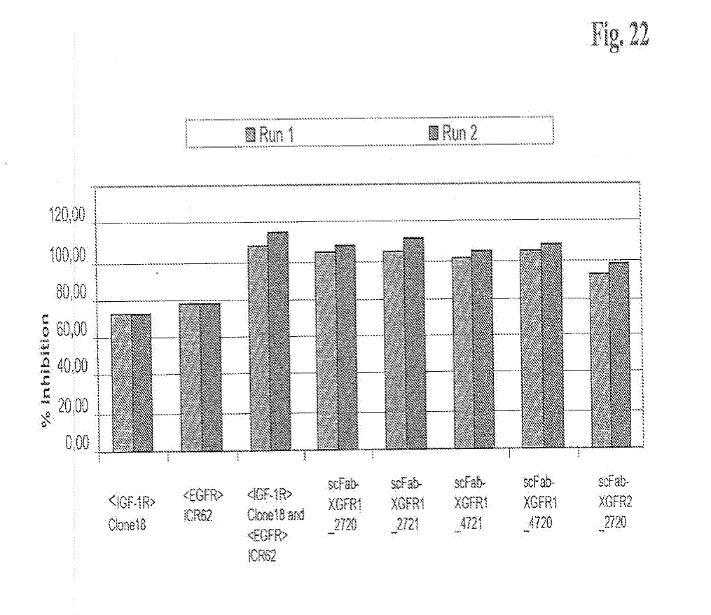

FIGS. 7a and 7b show that the application of <IGF-1R> HUMAB-Clone 18 strongly reduced the specific phosphorylation signal in an IGFR1-signalling assay but had no effect in a corresponding assay that measured EGFR-signaling. Vice versa, application of <EGFR>ICR62 reduced the specific phosphorylation signal in an EGFR-signalling assay by but showed no effect in a corresponding assay that measured IGF1R-signalling. The XGFR1 variants #2421, 3421, and 4421, when applied to the same assays at identical molarities, showed the same or better activities than the wildtype antibodies in both assays. Thus, XGFR1 molecules are capable of interfering with both signaling pathways.

### Example 6

### XGFR1-Mediated Growth Inhibition of Tumor Cell Lines In Vitro

The human anti-IGF-1R antibody <IGF-1R> HUMAB Clone 18 (DSM ACC 2587) inhibits the growth of tumor cell lines that express the IGF1R (WO 2005/005635). In a similar manner, the humanized rat anti-EGFR antibody <EGFR>ICR62 has been shown to inhibit the growth of tumor cell lines that express EGFR (WO 2006/082515). To evaluate the potential inhibitory activity of the different XGFR1 variants in growth assays of tumor cell lines, the degree of inhibition in H322M cells which express EGFR as well as IGF1R was analyzed.

H322M cells were cultured in RPMI 1640 supplemented with 0.5% FCS media on poly-HEMA (poly(2-hydroxyethylmethacrylate)) coated dishes to prevent adherence to the plastic surface. Under these conditions H322M cells form dense spheroids that grow three dimensionally (a property that is called anchorage independence). These spheroids resemble closely the three dimensional histoarchitecture and organization of solid tumors in-situ. Spheroid cultures were incubated for 5 days in the presence of increasing amounts of antibodies from 50 or 100 nM. The WST conversion assay was used to measure growth inhibition. When H322M spheroid cultures were treated with <IGF-1R> HUMAB-Clone18 an inhibition in growth could be observed.

FIG. 8 shows that the application of 50 nM <IGF-1R> HUMAB-Clone18 reduced the cell growth by 53%, and that the application of 50 nM <EGFR>ICR62 reduced the cell growth by 53% in the same assay.

The simultaneous application of both antibodies (at the same concentrations) resulted in a further decrease of cell viability to 26% (74% inhibition). This indicates that simultaneous interference with both RTK pathways has a more profound effect on tumor cell lines than the interference with just one pathway alone.

Application of various XGFR1-variants at molar concentration of 50 nM resulted in a higher growth inhibition that was more pronounced that that observed with single molecules alone at 50 nM concentrations.

In fact, at an antibody concentration of 50 nM, various XGFR1-variants showed an improved antiproliferative activity compared to the combination of the original <EGFR> and <IGF1R> antibodies at the doubled antibody concentration of 100 nM (50 nM <IGF-1R> HUMAB-Clone18 and 50 nM <EGFR>ICR62).

We conclude that XGFR1 molecules have a profoundly increased growth inhibitory activity compared to IgGs that interfere with either EGFR signaling or IGF1R signaling. Furthermore, if one compares the activity of XGFR1 molecules with the activity of the mixture of <IGF-1R> HUMAB-Clone18 and <EGFR>ICR62 antibodies, equal or better activity can be achieved at concentrations (molar and masses) that are clearly below that of the mixture.

TABLE 5
Antiproliferative activities (survival and inhibition) of bispecific
antibodies (XGFR -nomenclature) against H322M tumor cells.

| Antibody (concentration) | RLU | % survival | % inhibition |
|---|---|---|---|
| Medium | 32177 | 100 | 0 |
| Buffer | 32995 | 103 | −3 |
| IgG | 32847 | 102 | −2 |
| <IGF-1R> HUMAB-Clone18 | 15015 | 47 | 53 |
| (50 nM) | | | |
| <EGFR> ICR62 (50 nM) | 15163 | 47 | 53 |
| <IGF-1R> HUMAB-Clone18 | 8381 | 26 | 74 |
| (50 nM) + <EGFR> ICR62 | | | |
| (50 nM) (=100 nM antibody | | | |
| concentration) | | | |
| XGFR1-2321 (50 nM) | 8283 | 26 | 74 |
| XGFR1-2421 (50 nM) | 7356 | 23 | 77 |
| XGFR1-3321 (50 nM) | 8268 | 26 | 74 |
| XGFR1-3421 (50 nM) | 7989 | 25 | 75 |
| XGFR1-4321 (50 nM) | 16158 | 50 | 50 |
| XGFR1-4421 (50 nM) | 10668 | 33 | 67 |
| XGFR1-5321 (50 nM) | 14213 | 44 | 56 |
| XGFR1-5421 (50 nM) | 9506 | 30 | 70 |

### Example 7

### Preparation of the Glycoengineered Derivatives of XGFR1-2421, XGFR1-3421, XGFR1-4421 and XGFR1-5421

### XGFR1-2421-GE, XGFR1-3421-GE, XGFR1-4421-GE and XGFR1-5421-GE)

The resulting full antibody heavy and light chain DNA sequences were subcloned into mammalian expression vectors (one for the light chain and one for the heavy chain) under the control of the MPSV promoter and upstream of a synthetic polyA site, each vector carrying an EBV OriP sequence.

Antibodies were produced by co-transfecting HEK293-EBNA cells with the mammalian antibody heavy and light chain expression vectors using a calcium phosphate-transfection approach. Exponentially growing HEK293-EBNA cells were transfected by the calcium phosphate method. For the production of unmodified antibody, the cells were transfected only with antibody heavy and light chain expression vectors in a 1:1 ratio. For the production of the glycoengineered antibody, the cells were co-transfected with four plasmids, two for antibody expression, one for a fusion GnTIII polypeptide expression, and one for mannosidase II expression at a ratio of 4:4:1:1, respectively. Cells were grown as adherent monolayer cultures in T flasks using DMEM culture medium supplemented with 10% FCS, and were transfected when they were between 50 and 80% confluent. For the transfection of a T75 flask, 8 million cells were seeded 24 hours before transfection in 14 ml DMEM culture medium supplemented with FCS (at 10% V/V final), 250 μg/ml neomycin, and cells were placed at 37° C. in an incubator with a 5% CO2 atmosphere overnight. For each T75 flask to be transfected, a solution of DNA, CaCl₂ and water was prepared by mixing 47 μg total plasmid vector DNA divided equally between the light and heavy chain expression vectors, 235 μl of a 1M CaCl₂ solution, and adding water to a final volume of 469 μl. To this solution, 469 μl of a 50 mM HEPES, 280 mM NaCl, 1.5 mM Na2HPO4 solution at pH 7.05 were added, mixed immediately for 10 sec and left to stand at room temperature for 20 sec. The suspension was diluted with 12 ml of DMEM supplemented with 2% FCS, and added to the T75 in place of the existing medium. The cells were incubated at 37° C., 5% CO2 for about 17 to 20 hours, then medium was replaced with 12 ml DMEM, 10% FCS. The conditioned culture medium was harvested 5 to 7 days post-transfection centrifuged for 5 min at 1200 rpm, followed by a second centrifugation for 10 min at 4000 rpm and kept at 4° C.

The secreted antibodies were purified by Protein A affinity chromatography, followed by cation exchange chromatography and a final size exclusion chromatographic step on a Superdex 200 column (Amersham Pharmacia) exchanging the buffer to phosphate buffer saline and collecting the pure monomeric IgG1 antibodies. Antibody concentration was estimated using a spectrophotometer from the absorbance at 280 nm. The antibodies were formulated in a 25 mM potassium phosphate, 125 mM sodium chloride, 100 mM glycine solution of pH 6.7.

Glycoengineered variants of the humanized antibody were produced by co-transfection of the antibody expression vectors together with a GnT-III glycosyltransferase expression vector, or together with a GnT-III expression vector plus a Golgi mannosidase II expression vector. Glycoengineered antibodies were purified and formulated as described above for the non-glycoengineered antibodies. The oligosaccharides attached to the Fc region of the antibodies were analysed by MALDI/TOF-MS as described below.

Oligosaccharides were enzymatically released from the antibodies by PNGaseF digestion, with the antibodies being either immobilized on a PVDF membrane or in solution.

The resulting digest solution containing the released oligosaccharides either prepared directly for MALDI/TOF-MS analysis or was further digested with EndoH glycosidase prior to sample preparation for MALDI/TOF-MS analysis.

For all bispecific antibodies according to the invention, GE means glycoengineered.

### Example 8

### Binding to FcgRIIIa and ADCC-Competence of XGFR1 Molecules

The non-glycomodified humanized rat anti-EGFR antibody ICR62 (from WO 2006/082515) mediates its anti-tumor activity not only by interfering with RTK-mediated growth stimulatory signals, but also to a significant degree by inducing ADCC on tumor cells. In a similar manner, other antibodies, such as the anti-IGF-1R antibodies <IGF-1R> HUMAB Clone 18 are also capable of inducing ADCC. The degree of ADCC mediation by a given antibody depends not only on the antigen that is bound, but is also dependent on affinities of constant regions to the FcgRIIIa, which is known as the Fc receptor that triggers the ADCC reaction. Because ADCC is a desired mechanism for XGFR1 molecules, it is important that these molecules can bind FcgRIIIa in the same manner as ‘normal’ antibodies, and that these molecules have a good ADCC competence. For the analysis of binding of the various XGFR1 molecules to the bFcgRIIIa, we have applied a Biacore technology that has been previously established (References). By this technology, binding of XGFR1-molecules to recombinantly produced FcgRIIIa domains is assessed.

All surface plasmon resonance measurements were performed on a BIAcore 3000 instrument (GE Healthcare Biosciences AB, Sweden) at 25° C. The running and dilution buffer was PBS (1 mM KH2PO4, 10 mM Na2HPO4, 137 mM NaCl, 2.7 mM KCl), pH6.0, 0.005% (v/v) Tween20. The soluble human FcgRIIIa was diluted in 10 mM sodium-acetate, pH 5.0 and immobilized on a CM5 biosensor chip using the standard amine coupling kit (GE Healthcare Biosciences AB, Sweden) to obtain FcgRIIIa surface densities of approximately 1000 RU. HBS-P (10 mM HEPES, pH 7.4, 150 mM NaCl, 0.005% Surfactant P20; GE Healthcare Biosciences AB, Sweden) was used as running buffer during immobilization. XGFR bispecific antibodies were diluted with PBS, 0.005% (v/v) Tween20, pH6.0 to a concentration of 450 nM and injected over 3 minutes at a flow rate of 30 μl/minute. Then, the sensor chip was regenerated for 1 minute with PBS, pH8.0, 0.005% (v/v) Tween20. Data analysis was performed with the BIAevaluation software (BIAcore, Sweden).

The results of these experiments are summarized in Table 7.

TABLE 6
Binding affinities of bispecific antibodies
(XGFR -nomenclature) to FcγRIIIa and FcRn

| Molecule | Affinity to FcγRIIIa | Affinity to FcRn |
|---|---|---|
| XGFR1-2321 | yes | yes |
| XGFR1-2421 | yes | yes |
| XGFR1-3321 | yes | yes |
| XGFR1-3421 | yes | yes |
| XGFR1-4321 | yes | yes |
| XGFR1-4421 | yes | yes |
| XGFR1-5321 | yes | yes |
| XGFR1-5421 | yes | yes |
| <EGFR> ICR62 | yes | n.d |
| <IGF-1R> | n.d. | n.d. |
| HUMAB-Clone 18 | | |

These analyses reveal that binding towards FcgRIIIa of non-antigen bound XGFR1 molecules is indistinguishable from the binding of wild-type IgG1 molecules. Thus, these biochemical assays indicate full competency of non-antigen bound XGFR1-2421, and XGFR1-4421 to bind the ADCC-mediating receptor FcgRIIIa.

The repetition of these experiments using XGFR1 molecules in the presence of antigen revealed no effect on the ability of the soluble FcgRIIIa to bind.

Another set of such Biacore experiments was performed with XGFR1-molecules that have been glycomodified (see Example 7) by a previously described technology (Umana, P., et al. Nature Biotechnol. 17 (1999) 176-180 and WO 99/54342). This glycomodification increases the affinity of Fc-regions to the FcgRIIIa and thereby increases ADCC on target cells. A comparison of the FcgRIIIa-binding capability of non-antigen bound glycomodified XGFR1-molecules with that of non-antigen bound glycomodified wildtype IgG showed that non-antigen bound glycomodified XGFR1 molecules had increased binding affinity compared to the wildtype antibody.

TABLE 7
Binding affinities of bispecific antibodies
(XGFR -nomenclature) to FcγRIIIa and FcRn

| Molecule | Affinity to FcγRIIIa | Affinity to FcRn |
|---|---|---|
| XGFR1-2421-GE | yes | yes |
| XGFR1-3421-GE | yes | yes |
| XGFR1-4421-GE | yes | yes |
| XGFR1-5421-GE | yes | yes |

To analyze to what degree the binding competency of XGFR1-molecules to FcgRIIIa translates also into in-vitro ADCC activity towards tumor cells, we determined ADCC competency in cellular assays. For these assays, glycomodified derivatives of XGFR1-2421, XGFR1-3421, XGFR1-4421 and XGFR1-5421 (XGFR1-2421-GE, XGFR1-3421-GE, XGFR1-4421-GE and XGFR1-5421-GE) were prepared (see Example 6) and tested in the BIAcore ADCC-competence assay format that has been previously described and also the in-vitro ADCC assay as described below.

Human peripheral blood mononuclear cells (PBMC) were used as effector cells and were prepared using Histopaque-1077 (Sigma Diagnostics Inc., St. Louis, M063178 USA) following essentially the manufacturer's instructions. In brief, venous blood was taken with heparinized syringes from healthy volunteers. The blood was diluted 1:0.75-1.3 with PBS (not containing Ca⁺⁺ or Mg⁺⁺) and layered on Histopaque-1077. The gradient was centrifuged at 400×g for 30 min at room temperature (RT) without breaks. The interphase containing the PBMC was collected and washed with PBS (50 ml per cells from two gradients) and harvested by centrifugation at 300×g for 10 minutes at RT. After resuspension of the pellet with PBS, the PBMC were counted and washed a second time by centrifugation at 200×g for 10 minutes at RT. The cells were then resuspended in the appropriate medium for the subsequent procedures.

The effector to target ratio used for the ADCC assays was 25:1 for PBMC. The effector cells were prepared in AIM-V medium at the appropriate concentration in order to add 50 μl per well of round bottom 96 well plates. Target cells were human EGFR/IGFR expressing cells (e.g., H322M, A549, or MCF-7) grown in DMEM containing 10% FCS. Target cells were washed in PBS, counted and resuspended in AIM-V at 0.3 million per ml in order to add 30,000 cells in 100 μl per microwell. Antibodies were diluted in AIM-V, added in 50 μl to the pre-plated target cells and allowed to bind to the targets for 10 minutes at RT. Then the effector cells were added and the plate was incubated for 4 hours at 37° C. in a humidified atmosphere containing 5% CO2. Killing of target cells was assessed by measurement of lactate dehydrogenase (LDH) release from damaged cells using the Cytotoxicity Detection kit (Roche Diagnostics, Rotkreuz, Switzerland). After the 4-hour incubation the plates were centrifuged at 800×g. 100 μl supernatant from each well was transferred to a new transparent flat bottom 96 well plate. 100 μl color substrate buffer from the kit were added per well. The Vmax values of the color reaction were determined in an ELISA reader at 490 nm for at least 10 min using SOFTmax PRO software (Molecular Devices, Sunnyvale, Calif. 94089, USA). Spontaneous LDH release was measured from wells containing only target and effector cells but no antibodies. Maximal release was determined from wells containing only target cells and 1% Triton X-100. Percentage of specific antibody-mediated killing was calculated as follows: ((x−SR)/(MR−SR)*100, where x is the mean of Vmax at a specific antibody concentration, SR is the mean of Vmax of the spontaneous release and MR is the mean of Vmax of the maximal release.

In these assays, the ADCC competency was also compared to that of the glycomodified wild-type antibodies. The results of these assays showed excellent ADCC competence for the glycomodified XGFR1-3421-GE/XGFR1-4421-GE/XGFR1-5421-GE (see FIG. 9).

### Example 9

### Expression & Purification of Bispecific <EGFR-IGF1R> Antibody scFab-XGFR1 Molecules

Light and heavy chains of the corresponding bispecific antibodies were constructed in expression vectors carrying pro- and eukaryotic selection markers. These plasmids were amplified in E. coli, purified, and subsequently transfected for transient expression of recombinant proteins in HEK293F cells (utilizing Invitrogen's freesyle system). After 7 days, HEK 293 cell supernatants were harvested and purified by protein A and size exclusion chromatography. Homogeneity of all bispecific antibody constructs was confirmed by SDS-PAGE under non reducing and reducing conditions. Under reducing conditions (FIG. 15), polypeptide chains carrying C- and N-terminal scFab fusions showed upon SDS-PAGE apparent molecular sizes analogous to the calculated molecular weights. Expression levels of all constructs were analysed by Protein A HPLC and were similar to expression yields of ‘standard’ IgGs, or in some cases somewhat lower. Average protein yields were between 1.5 and 10 mg of protein per liter of cell-culture supernatant in such non-optimized transient expression experiments (FIGS. 13 and 14).

HP-Size exclusion chromatography analysis of the purified proteins showed some tendency to aggregate for recombinant molecules. To address the problems with aggregation of such bispecific antibodies, disulfide-stabilization between VH and VL of the additional binding moieties was applied. For that we introduced single cysteine replacements within VH and VL of the scFab at defined positions (positions VH44/VL100 according to the Kabat numbering scheme). These mutations enable the formation of stable interchain disulfides between VH and VL, which in turn stabilize the resulting disulfide-stabilized scFab module. Introduction of the VH44/VL100 disulfides in scFabs did not significantly interfere with protein expression levels and in some instance even improved expression yields (see FIGS. 13 and 14).

The bispecific antibodies were expressed by transient transfection of human embryonic kidney 293-F cells using the FreeStyle™ 293 Expression System according to the manufacturer's instruction (Invitrogen, USA). Briefly, suspension FreeStyle™ 293-F cells were cultivated in FreeStyle™ 293 Expression medium at 37° C./8% CO₂ and the cells were seeded in fresh medium at a density of 1-2×10⁶ viable cells/ml on the day of transfection. The DNA-293fectin™ complexes were prepared in Opti-MEM® I medium (Invitrogen, USA) using 333 μl of 293fectin™ (Invitrogen, Germany) and 250 μg of heavy and light chain plasmid DNA in a 1:1 molar ratio for a 250 ml final transfection volume. Recombinant antibody derivative containing cell culture supernatants were clarified 7 days after transfection by centrifugation at 14000 g for 30 minutes and filtration through a sterile filter (0.22 μm). Supernatants were stored at −20° C. until purification.

The secreted antibody derivatives were purified from the supernatant in two steps by affinity chromatography using Protein A-Sepharose™ (GE Healthcare, Sweden) and Superdex200 size exclusion chromatography. Briefly, the bispecific and trispecific antibody containing clarified culture supernatants were applied on a HiTrap ProteinA HP (5 ml) column equilibrated with PBS buffer (10 mM Na₂HPO₄, 1 mM KH₂PO₄, 137 mM NaCl and 2.7 mM KCl, pH 7.4). Unbound proteins were washed out with equilibration buffer. The antibody derivatives were eluted with 0.1 M citrate buffer, pH 2.8, and the protein containing fractions were neutralized with 0.1 ml 1 M Tris, pH 8.5. Then, the eluted protein fractions were pooled, concentrated with an Amicon Ultra centrifugal filter device (MWCO: 30 K, Millipore) to a volume of 3 ml and loaded on a Superdex200 HiLoad 120 ml 16/60 gel filtration column (GE Healthcare, Sweden) equilibrated with 20 mM Histidin, 140 mM NaCl, pH 6.0. Monomeric antibody fractions were pooled, snap-frozen and stored at −80° C. Part of the samples were provided for subsequent protein analytics and characterization. Exemplary SDS-PAGE analyses of purified proteins and profiles of HP-Size Exclusion Chromatography (SEC) of bispecific antibody derivatives are shown in FIGS. 15 and 16.

FIGS. 13 and 14 lists the expression yields that were observed in transient expression systems: All designed antibody derivatives could be expressed and purified in sufficient amounts for further analyses. The expression yield per liter supernatant ranged from less than 1 mg to >30 mg. For example, scFab-XGFR1-2720 had a final yield after purification of less than 1 mg whereas scFab-XGFR1-2721 had a final yield of 13.8 mg. This difference shows also the positive influence of the VH44-VL100 disulfide stabilization on expression yields that we observed for some proteins.

### Example 10

### Stability of Bispecific <EGFR-IGF1R> Antibody scFab-XGFR1 Molecules In Vitro Stability and Aggregation Tendency of Bispecific <EGFR-IGF1R> Antibody scFab Molecules

HP-Size exclusion chromatography analysis was performed to determine the amounts of aggregates that are present in preparation of recombinant antibody derivatives. For that, bispecific antibody samples were analyzed by high-performance SEC on an UltiMate 3000 HPLC system (Dionex) using a Superdex 200 analytical size-exclusion column (GE Healthcare, Sweden). FIG. 16 shows an example of these analyses. Aggregates appear as a separate peak or shoulder before the fractions that contain the monomeric antibody derivative. For this work, we define desired ‘monomeric’ molecules to be composed of 2 heterodimers of heavy and light chains-with scFabs connected to either of both. The integrity of the amino acid backbone of reduced bispecific antibody light and heavy chains and -fusion proteins was verified by NanoElectrospray Q-TOF mass spectrometry after removal of N-glycans by enzymatic treatment with Peptide-N-Glycosidase F (Roche Molecular Biochemicals). HP-Size exclusion chromatography analysis of the purified proteins under different conditions (varying concentration and time) showed—compared to normal IgGs—a somewhat increased tendency to aggregate for molecules that contained scFabs. This aggregation tendency that we observed for some molecules could be ameliorated by introduction of the VH44/VL100 interchain disulfide bond in scFab modules.

### Example 11

### Binding of Bispecific <EGFR-IGF1R> Antibody scFab-Molecules to the RTKs EGFR and IGF1R

The binding of the scFab modules and of the antigen-binding sites of the retained in the full length IgG-module of the different bispecific antibody formats scFab-XGFR were compared to the binding of the ‘wildtype’ IgGs from which the binding modules and bispecific antibodies were derived. These analyses were carried out by applying Surface Plasmon Resonance (Biacore), as well as a cell-ELISA.

The binding properties bispecific <IGF-1R-EGFR> antibodies were analyzed by surface plasmon resonance (SPR) technology using a Biacore T100 instrument (GE Healthcare Bio-Sciences AB, Uppsala). This system is well established for the study of molecule interactions. It allows a continuous real-time monitoring of ligand/analyte bindings and thus the determination of association rate constants (ka), dissociation rate constants (kd), and equilibrium constants (KD) in various assay settings. SPR-technology is based on the measurement of the refractive index close to the surface of a gold coated biosensor chip. Changes in the refractive index indicate mass changes on the surface caused by the interaction of immobilized ligand with analyte injected in solution. If molecules bind to immobilized ligand on the surface the mass increases, in case of dissociation the mass decreases.

Capturing anti-human IgG antibody was immobilized on the surface of a C1 biosensorchip using amine-coupling chemistry. Flow cells were activated with a 1:1 mixture of 0.1 M N-hydroxysuccinimide and 0.1 M 3-(N,N-dimethylamino)propyl-N-ethylcarbodiimide at a flow rate of 5 μl/min Anti-human IgG antibody was injected in sodium acetate, pH 5.0 at 5 μg/ml, which resulted in a surface density of approximately 200 RU. A reference control flow cell was treated in the same way but with vehicle buffers only instead of the capturing antibody. Surfaces were blocked with an injection of 1 M ethanolamine/HCl pH 8.5. The bispecific antibodies were diluted in HBS-P and injected at a flow rate of 5 μl/min. The contact time (association phase) was 1 min for the antibodies at a concentration between 1 and 5 nM. EGFR-ECD was injected at increasing concentrations of 1.2, 3.7, 11.1, 33.3, 100 and 300 nM, IGF-1R at concentrations of 0.37, 1.11, 3.33, 10, 30 and 90 nM. The contact time (association phase) was 3 min, the dissociation time (washing with running buffer) 5 min for both molecules at a flowrate of 30 μl/min. All interactions were performed at 25° C. (standard temperature). The regeneration solutions of 0.85% phosphoric acid and 5 mM sodium hydroxide were injected each for 60 s at 5 μl/min flow to remove any non-covalently bound protein after each binding cycle. Signals were detected at a rate of one signal per second. Samples were injected at increasing concentrations.

Exemplary simultaneous binding of an bispecific antibody <IGF-1R-EGFR> antibodies to EGFR and IGF1R is shown in FIG. 17a-d.

TABLE 8
Affinities (KD) of bispecific antibodies (scFab-XGFR1_2720
and scFab-XGFR2_2720) to EGFR and IGF-1R

| | KD value | KD value |
|---|---|---|
| Molecule | (Affinity to EGFR) | (Affinity to IGF-1R) |
| scFab-XGFR1_2720 | 2 nM | 2 nM |
| scFab-XGFR2_2720 | 0.5 nM | 11 nM |
| <IGF-1R> Clone 18 | n.a. | 2 nM |
| <EGFR> ICR62 | 0.5 nM | n.a. |

FACS-based binding-and competition-analyses on cultured cells can also be applied to assess the binding capability of bispecific antibody derivatives to RTKs that are exposed on cell surfaces. FIG. 18 shows the experimental set-up that we used to test binding capabilities of scFab containing bispecific XGFR derivatives on A549 cancer cells. For these cellular competition assays, A549 cells which express the antigens EGFR as well IGF1R were detached and counted. 1.5×10e5 cells were seeded per well of a conical 96-well plate. Cells were spun down (1500 rpm, 4° C., 5 min) and incubated for 45 min on ice in 50 μL of a dilution series of the respective bispecific antibody in PBS with 2% FCS (fetal calf serum) containing 1 μg/mL of Alexa647-labeled IGFIR-specific antibody. Cells were again spun down and washed twice with 200 μL PBS containing 2% FCS. Finally, cells were resuspended in BD CellFix solution (BD Biosciences) and incubated for at least 10 min on ice. Mean fluorescence intensity (mfi) of the cells was determined by flow cytometry (FACS Canto). Mfi was determined at least in duplicates of two independent stainings. Flow cytometry spectra were further processed using the FlowJo software (TreeStar). Half-maximal binding was determined using XLFit 4.0 (IDBS) and the dose response one site model 205.

The results of these assays which are shown in FIG. 19a-c demonstrate binding functionality of the bispecific scFab containing antibody derivatives on surfaces of tumor cells. For example, the IC50 in competition experiments of the bispecific antibody derivative scFab-XGFR12721 was 0.11 μg/ml whereas the IC50 of the monospecific antibody was >50% higher (0.18 μg/ml). This increased activity in competition assays of the bispecific scFab-XGFR 2721 derivative compared to the parent antibody suggests that the bispecific molecule binds better to cell surfaces than the monospecific antibody.

### Example 12

### Downregulation of EGFR- as Well as IGF-1-by Bispecific <EGFR-IGF-1R> Antibody scFab-XGFR Molecules

The human anti-IGF-1R antibodies <IGF-1R> HUMAB Clone 18 (DSM ACC 2587) inhibits IGFR1-signaling and the humanized rat anti-EGFR antibody <EGFR>ICR62 inhibits the signaling by EGFR. To evaluate the potential inhibitory activity of the different scFab-XGFR1 variants, the degree of downregulation of the receptor from both was analyzed.

In order to detect effects of the antibody of the invention on the amount of IGF-I receptor (IGF-IR) in tumor cells, time-course experiments and subsequent ELISA analysis with IGF-IR and EGFR specific antibodies were performed.

A 6 well plate were inoculated with 1 ml per well human tumor cells (H322M, 5×10⁵ cells/ml) in RPMI 1640 supplemented with 10% FCS (PAA, Cat. No. E15-039) and 1% PenStrep. 3 ml medium were added to each well and the cells were cultivated for 24 hours at 37° C. and 5% CO₂.

The medium was carefully removed and replaced by 2 ml 100 nM XGFR antibodies diluted in RPMI-VM medium. In control wells, medium was replaced by either medium and buffer without antibody and medium with control antibodies (<IGF-1R> HUMAB Clone 18 and <EGFR>ICR62 final concentration 100 nM). Cells were incubated at 37° C. and 5% CO₂ and individual plates were taken out for further processing after 24 hours.

The medium was carefully removed by aspiration and the cell were washed with 1 ml PBS. 300 μl/well of cold MES-lysis buffer was added (MES, 10 mM Na₃VO₄, and Complete® protease inhibitor). After one hour the cells were detached on ice using a cell scraper (Corning, Cat. No. 3010) and the well contents transferred to Eppendorf reaction tubes. Cell fragments were removed by centrifugation for 10 minutes at 13000 rpm and 4° C.

### For EGFR Detection

The 96 well microtitreplates (MTP) were prepared according to the protocol (DuoSet ELISA for Human EGFR, RnD systems Cat. No. DY231). The Human EGFR goat antibody 144 μg/ml in PBS was diluted 1:180 in PBS and 100 μl/well was added to the MTP. The MTP was incubated overnight at room temperature with agitation. The plates were washed 3 times with PBS supplemented with 0.1% Tween®20 and blocked with 300 μl/well of PBS, 3% BSA and 0.1% Tween®20 solution for 1 hour (h) at room temperature (RT) with agitation. The plates were washed 3 times with PBS supplemented with 0.1% Tween® 20.

The amount of protein in the cell lysates was determined using the BCA Protein Assay kit (Pierce), the cell lysates were then adjusted to a protein concentration of 0.04 mg/ml with MES-lysis buffer supplemented with 100 mM Na₃VO₄ 1:100 and Complete® protease inhibitor 1:20 and 100 μl per well of the lysate was added to the pre-prepared MTP. For background measurement 100 μl lysis buffer was added to the well in the MTP.

A second cell lysate concentration was used at 0.025 mg/ml the lysate was dilute 1:2 and 100 μl was added per well to the pre-prepared MTP. The MTP were incubated for a further 2 hour at RT with agitation and then washed 3 times with PBS with 0.1% Tween®20 solution.

The detection antibody for EGFR was human EGFR goat biotinylated antibody at a concentration of 36 μg/ml diluted 1:180 in PBS, 3% BSA and 0.2% Tween®20, 100 μl per well was added and incubated at RT for 2 hours with agitation. The MTP was then washed three times with 200 μl per well of PBS with 0.1% Tween®20 solution. Then Streptavidin-HRP 1:200 in PBS, 3% BSA and 0.2% Tween®20 100 μl per well was added and incubated with agitation for 20 minutes at RT. The plate was then washed six times with PBS with 0.1% Tween®20 solution. 100 μl per well of 3,3′-5,5′-Tetramethylbenzidin (Roche, BM-Blue ID-No.: 11484581) was added and incubated for 20 minutes at RT with agitation. The color reaction was stopped by adding 25 μl per well of 1M H₂SO₄ and incubating for a further 5 minutes at RT. The absorbance was measured at 450 nm.

### For IGF-1R Detection

The streptavidin-MTP (Roche ID. No.: 11965891001) was prepared by adding 100 μl per well of the antibody AK1a-Biotinylated (Genmab, Denmark) which was diluted 1:200 in PBS, 3% BSA and 0.2% Tween®20. The streptavidin-MTP was incubated for 1 hour at RT with agitation and then washed three times with 200 μl per well of PBS with 0.1% Tween®20 solution.

The amount of protein in the cell lysates was determined using the BCA Protein Assay kit (Pierce), the cell lysates were then adjusted to a protein concentration of 0.3 mg/ml with 50 mM Tris pH 7.4, 100 mM Na₃VO₄ 1:100 and Complete® protease inhibitor 1:20 and 100 μl per well of the lysate was added to the pre-prepared streptavidin-MTP.

A second cell lysate concentration was used at 0.15 mg/ml the lysate was dilute and 100 μl was added per well to the pre-prepared streptavidin-MTP. For background measurement 100 μl lysis buffer was added to the well in the streptavidin-MTP.

The MTP were incubated for a further 1 hour at RT with agitation and then washed 3 times with PBS with 0.1% Tween®20 solution.

The detection antibody for IGF-1R was human IGF-1Rβ rabbit antibody (Santa Cruz Biotechnology, Cat. No. sc-713) diluted 1:750 in PBS, 3% BSA and 0.2% Tween®20, 100 μl per well was added and incubated at RT for 1 hour with agitation. The MTP was then washed three times with 200 μl per well of PBS with 0.1% Tween®20 solution. The secondary antibody was then added rabbit IgG-POD (Cell signaling Cat. No. 7074) 1:4000 in PBS, 3% BSA and 0.2% Tween®20, 100 μl was added per well and incubated with agitation for 1 hour at RT. The plate was then washed six times with PBS with 0.1% Tween®20 solution. 100 μl per well of 3,3′-5,5′-Tetramethylbenzidin (Roche, BM-Blue ID-No.: 11484581) was added and incubated for 20 minutes at RT with agitation. The colour reaction was stopped by adding 25 μl per well of 1M H₂SO₄ and incubating for a further 5 minutes at RT. The absorbance was measured at 450 nm.

The results of the receptor downregulation detection by the bispecific scFab containing XGFR molecules compared to the parent monospecific antibodies <EGFR>ICR62 and <IGF-1R> HUMAB-Clone 18 in H322M cells is shown in FIGS. 20 and 21. The bispecific antibodies scFab-XGFR downregulate both EGFR- as well as the IGF1R. This shows that full functionality (biological functionality) and phenotype mediation of the binding modules is retained. FIG. 21 also indicates that, surprisingly, the bispecific antibodies scFab-XGFR 2720 showed an improved downregulation of EGFR compared to the parent <EGFR>ICR62 antibody alone.

The fact that scFab containing XGFR1 variants when applied to the same assays at identical molarities, showed the same or better activities than the wildtype antibodies indicates that scFab-XGFR1 molecules are capable of interfering with both signaling pathways.

### Example 13

### scFab-XGFR1 and scFab-XGFR2-Mediated Growth Inhibition of Tumor Cell Lines In Vitro

The human anti-IGF-1R antibody <IGF-1R> HUMAB Clone 18 (DSM ACC 2587) inhibits the growth of tumor cell lines that express the IGF1R (WO 2005/005635). In a similar manner, the humanized rat anti-EGFR antibody <EGFR>ICR62 has been shown to inhibit the growth of tumor cell lines that express EGFR (WO 2006/082515). To evaluate the potential inhibitory activity of the different scFab-XGFR1 variants in growth assays of tumor cell lines, the degree of inhibition in H322M cells which express EGFR as well as IGF1R was analyzed.

H322M cells (5000 cells/well) were cultured in RPMI 1640 media supplemented with 10% FCS on poly-HEMA (poly(2-hydroxyethylmethacrylate)) coated dishes to prevent adherence to the plastic surface. Under these conditions H322M cells form dense spheroids that grow three dimensionally (a property that is called anchorage independence). These spheroids resemble closely the three dimensional histoarchitecture and organization of solid tumors in-situ. Spheroid cultures were incubated for 7 days in the presence of 100 nM antibodies. The Celltiter Glow luminescence assay was used to measure growth inhibition. When H322M spheroid cultures were treated with <IGF-1R> HUMAB-Clone18 an inhibition in growth could be observed.

FIG. 22 shows that the application of 100 nM <IGF-1R> HUMAB-Clone18 reduced the cell growth by 72%, and that the application of 100 nM <EGFR>ICR62 reduced the cell growth by 77% in the same assay. The simultaneous application of both antibodies (both at the same concentrations of 100 nM) resulted in a complete decrease of cell viability (100% inhibition). This indicates that simultaneous interference with both RTK pathways has a more profound effect on tumor cell lines than the interference with just one pathway alone. Application of various scFab-XGFR1-variants at molar concentration of 100 nM resulted in a higher growth inhibition that was more pronounced that that observed with single molecules alone. In fact, at an antibody concentration of 100 nM, various scFab-XGFR1-variants showed complete (100%) inhibition of cell growth, while application of single modules caused only partial inhibition.

We conclude that scFab-XGFR1 molecules have a profoundly increased growth inhibitory activity compared to IgGs that solely interfere with either EGFR signaling or IGF1R signaling.

### Example 14

### Expression & Purification of Bispecific, Bivalent Domain Exchanged <EGFR-IGF1R> Antibody Molecules Cross-Mab (VH/VL) (VH/VL Domain Exchange) or Cross-Mab (CH/CL) (CH/CL Domain Exchange)

Analogous to the procedures described in Example 1 and 9, bispecific, bivalent domain exchanged <EGFR-IGF1R> antibody molecules Cross-Mab (VH/VL) (VH/VL exchange as described in WO 2009/080252) and Cross-Mab (CH/CL) (CH/CL exchange as described in WO 2009/080253) were expressed and purified. Both bispecific <EGFR-IGF-1R> antibodies were based on the heavy chain variable domains of SEQ ID NO: 8, and the light chain variable domains of SEQ ID NO: 10 (derived from humanized <EGFR>ICR62) as first antigen-binding site binding to EGFR, and on the heavy chain variable domains of SEQ ID NO: 23, and the light chain variable domains of SEQ ID NO: 25 (derived from the human anti-IGF-1R antibodies <IGF-1R> HUMAB Clone 18 (DSM ACC 2587)) as second antigen-binding site binding to IGF-1R.

The expression yields after by affinity chromatography using Protein A-Sepharose™ (GE Healthcare, Sweden) and Superdex200 size exclusion chromatography were 29.6 mg/L for the Cross-Mab (VH/VL) and 28.2 mg/L for the Cross-Mab (CH/CL).

The relevant full (partially modified) light and heavy chains amino acid sequences of the corresponding bispecific antibodies are given in SEQ ID NO: 30-33 for the Cross-Mab (VH/VL) and in SEQ ID NO: 34-37 for the Cross-Mab (CH/CL).

### Example 15

### Downregulation of EGFR- as Well as IGF-1R-by Bispecific, Bivalent Domain Exchanged <EGFR-IGF1R> Antibody Molecules Cross-Mab (VH/VL) or Cross-Mab (CH/CL)

Analogously to Example 12 the downregulation of EGFR- as well as IGF-1R on H322M tumor cells by bispecific, bivalent domain exchanged <EGFR-IGF1R> antibody molecules Cross-Mab (VH/VL) (VH/VL exchange) and Cross-Mab (CH/CL) (CH/CL exchange) of Example 14 was determined.

Downregulation of EGFR of both bispecific, bivalent domain exchanged <EGFR-IGF1R> antibodies Cross-Mab (VH/VL) and Cross-Mab (CH/CL) was similar (Cross-Mab (VH/VL) ca. 41%) or slightly higher (Cross-Mab (VH/VL) ca 49%) when compared to the downregulation of monospecific <EGFR>ICR62 (ca 41%; at 9.38 μg Protein/ml).

Downregulation of IGF-1R of both bispecific, bivalent domain exchanged <EGFR-IGF1R> antibodies Cross-Mab (VH/VL) and Cross-Mab (CH/CL) was surprisingly significant lower (Cross-Mab (VH/VL) ca 17%) (Cross-Mab (VH/VL) ca 20%) when compared to the downregulation of monospecific <IGF-1R> HUMAB-Clone18 (ca 85%; at 75 μg Protein/ml).

### Example 16

### Tumor Growth Inhibition of H322M Tumor Cell Lines In Vitro by Bispecific, Bivalent Domain Exchanged <EGFR-IGF1R> Antibody Molecules Cross-Mab (VH/VL) or Cross-Mab (CH/CL)

Analogously to Example 13 the tumor growth inhibition of H322M tumor cells of bispecific, bivalent domain exchanged <EGFR-IGF1R> antibody molecules Cross-Mab (VH/VL) (VH/VL exchange) and Cross-Mab (CH/CL) (CH/CL exchange) of Example 14 was determined

At 100 nM the monospecific antibodies <IGF-1R> HUMAB-Clone18 reduced the cell growth by 75%, and that the application of 100 nM <EGFR>ICR62 reduced the cell growth by 89%.

The simultaneous application of both antibodies (both at the same concentrations of 100 nM which results in 200 nM antibody concentration in total resulted in a complete decrease of cell viability (≧100% inhibition).

The bispecific, bivalent domain exchanged <EGFR-IGF1R> antibody molecules Cross-Mab (VH/VL) and Cross-Mab (CH/CL) (at concentrations of only 100 nM) each showed also separately complete (≧100%) inhibition of cell growth.

This indicates that a bispecific antibody according to the invention can completely inhibit tumor cell growth at a lower antibody concentration than the combination of the corresponding monospecific parent antibodies, while the monospecific parent antibodies alone only caused partial inhibition.

### Example 17

### Expression & Purification of Bispecific, Bivalent ScFab-Fc Fusion <EGFR-IGF1R> Antibody Molecule scFab-Fc

Analogous to the procedures described in Example 1 and 9, bispecific, bivalent ScFab-Fc fusion <EGFR-IGF1R> antibody scFab-Fc was expressed and purified. This bispecific <EGFR-IGF-1R> antibody is also based on the heavy chain variable domains of SEQ ID NO: 8, and the light chain variable domains of SEQ ID NO: 10 (derived from humanized <EGFR>ICR62) as first antigen-binding site binding to EGFR, and on the heavy chain variable domains of SEQ ID NO: 23, and the light chain variable domains of SEQ ID NO: 25 (derived from the human anti-IGF-1R antibodies <IGF-1R> HUMAB Clone 18 (DSM ACC 2587)) as second antigen-binding site binding to IGF-1R.

The expression yields after by affinity chromatography using Protein A-Sepharose™ (GE Healthcare, Sweden) and Superdex200 size exclusion chromatography were 29.7 mg/L for the scFab-Fc.

TABLE 10
Yield of bispecific, bivalent ScFab-Fc fusion <EGFR-IGF1R>
antibody molecule scFab-Fc after expression and purification

| | |
|---|---|
| Protein A | SEC |

| Supernatant | Yield | Mono. | Yield | Mono. |
|---|---|---|---|---|
| 1.0 L | 32.5 mg | 88% | 29.7 mg | 100% |

The relevant full (modified) heavy chains amino acid sequences of the bispecific antibody scFab-Fc are given in SEQ ID NO: 38-39

### Example 18

### Expression & Purification of Bispecific, Bivalent ScFab-Fc Fusion <EGFR-IGF1R> Antibody Molecules

Analougously to Example 12 the downregulation of EGFR- as well as IGF-1R on H322M tumor cells caused by bispecific, bivalent ScFab-Fc fusion <EGFR-IGF1R> antibody of Example 17 is determined.

### Example 19

### Tumor Growth Inhibition of Tumor Cell Lines In Vitro Bispecific, Bivalent ScFab-Fc Fusion <EGFR-IGF1R> Antibody Molecules

Analogously to Example 13 the tumor growth inhibition of H322M tumor cells of bispecific, bivalent ScFab-Fc fusion <EGFR-IGF1R> antibody of Example 17 is determined.

### Example 20

### Survival Analysis in the Orthotopic A549 Xenograft Model

### Cell Culture

A549 adenocarcinoma cells (NSCLC) were originally obtained from ATCC and after expansion deposited in the internal cell bank. Tumor cell line was routinely cultured in DMEM medium (GIBCO, Switzerland) supplemented with 10% fetal bovine serum (Invitrogen, Switzerland) and 2 mM L-glutamine (GIBCO, Switzerland) at 37° C. in a water-saturated atmosphere at 5% CO2. Culture passage was performed with trypsin/EDTA 1× (GIBCO, Switzerland) splitting every third day. Passage 10 was used for injection.

### Animals

SCID beige female mice; age 8-9 weeks at start of experiment (purchased from Charles River, Sulzfeld, Germany) were maintained under specific-pathogen-free condition with daily cycles of 12 h light/12 h darkness according to committed guidelines (GV-Solas; Felasa; TierschG). Experimental study protocol was reviewed and approved by local government (P 2005086). After arrival animals were maintained for one week to get accustomed to new environment and for observation. Continuous health monitoring was carried out on regular basis.

### Tumor Cell Injection

At day of injection, A549 tumor cells were harvested using trypsin-EDTA (Gibco, Switzerland) from culture flasks (Greiner Bio-One) and transferred into 50 ml culture medium, washed once and resuspended in AIM V (Gibco, Switzerland). After an additional washing with AIM V, cell concentration was determined using a cell counter. For injection of A549 cells, the final titer was adjusted to 5.0×10⁶ cells/ml. Subsequently 200 μl of this mixture was injected into the lateral tail vein of the mice using a 1.0 ml tuberculin syringe (BD Biosciences, Germany)).

### Treatment

Animal treatment started two weeks after tumor cell inoculation with 10 animals per group. The bispecific anti-EGFR/anti-IGF1R antibodies XGFR1-4421 GE, XGFR1-2421 GE, XGFR1-3421 GE, <EGFR>ICR62GE, <IGF-1R> HUMAB-Clone18, and the corresponding vehicle were administered i.v. once weekly at the indicated dosage. A monthly dose was administered till the end of the experiment. The antibody dilutions were prepared freshly from stock before use.

TABLE 11
Study design of Survival analysis in
the orthotopic A549 xenograft model

| | No. of | | | Route/Mode | No. of |
|---|---|---|---|---|---|
| | ani- | | Dose | of adminis- | treat- |
| Group | mals | Compound | mg/kg | tration | ments |
| 1 | 10 | Vehicle (+2 | — | i.v. | 3 |
| | | scouts) | | | |
| 2 | 10 | <EGFR>ICR62 | 10 mg/kg + | i.v. | 3 |
| | | GE and | 10 mg/kg | | |
| | | <IGF-1R> | | | |
| | | HUMAB- | | | |
| | | Clone18 | | | |
| 3 | 10 | XGFR1-4421 | 13.6 mg/kg | i.v. | 3 |
| | | GE | | | |
| 4 | 10 | XGFR1-2421 | 13.6 mg/kg | i.v. | 3 |
| | | GE | | | |
| 5 | 10 | XGFR1-3421 | 13.6 mg/kg | i.v. | 3 |
| | | GE | | | |
| 6 | 6 | <EGFR>ICR62 | 25 mg/kg | i.v. | 3 |
| | | GE | | | |

GE = glycoengineered

### Monitoring

Animals were controlled daily for clinical symptoms and detection of adverse effects namely, respiratory distress, impaired motility and scruffy fur. Study exclusion criteria for animals were described and approved in the corresponding project license.

### Identification/Staging

Mice were randomly distributed at staging. Animals were housed in M3 size cages.

### Autopsy

Mice were sacrificed according to the termination criteria (scruffy fur, arched back, impaired locomotion). From all animal lung tumors were harvested for subsequent histopathological analysis (PFA, frozen).

### Survival Analysis

Survival data contain duration times until the occurrence of a specific event and are sometimes referred to as time-to-event data. The event can e.g. be the death of a patient. If the event does not occur before the end of a study for an observation or when a study object leaves the study before the event occurs, the observation is said to be censored. Then the exact survival time is unknown, but it is known that it is greater than the specified value.

Survival data need to be analyzed with specialized methods, because they have specialized non-normal distributions, like the exponential or Weibull. Furthermore, the censored observations cannot be ignored without biasing the analysis.

Kaplan-Meier curves give an estimation of the survival functions for one or more groups of right-censored data.

TABLE 12
Quantiles -Summary Median survival

| Group | Median survival Time |
|---|---|
| Vehicle | 68 |
| <EGFR>ICR62 GE | 136 |
| <EGFR>ICR62 GE and <IGF-1R> | 207 |
| HUMAB-Clone18 | |
| XGFR1-4421 GE | 212 |
| XGFR1-2421 GE | 207 |
| XGFR1-3421 GE | 212 |

GE = glycoengineered

The quantiles table shows the median survival time. From Table Y follows that the median survival time in days of the treatment with bispecific <EGFR-IGF1R> antibodies XGFR1-4421 GE, XGFR1-2421 GE, XGFR1-3421 GE is higher when compared to the treatment with monospecific <EGFR>ICR62GE, and is higher or at least the same when compared to the treatment with the combination of <EGFR>ICR62GE and <IGF-1R> HUMAB-Clone18.

### Example 21

### Expression & Purification, In Vitro and In Vivo Properties of Bispecific, Bivalent ScFab-Fc Fusion <EGFR-IGF1R> Antibody Molecules N-scFabSS-Salt-Bridge-s3 and N-scFabSS-Salt-Bridge-w3C

Analogous to the procedures described in Examples 17, 1 and 9, bispecific, bivalent ScFab-Fc fusion <EGFR-IGF1R> antibody molecules N-scFabSS-salt-bridge-s3 and N-scFabSS-salt-bridge-w3C are expressed and purified. These bispecific <EGFR-IGF-1R> antibodies are also based on the heavy chain variable domains of SEQ ID NO: 8, and the light chain variable domains of SEQ ID NO: 10 (derived from humanized <EGFR>ICR62) as first antigen-binding site binding to EGFR, and on the heavy chain variable domains of SEQ ID NO: 23, and the light chain variable domains of SEQ ID NO: 25 (derived from the human anti-IGF-1R antibodies <IGF-1R> HUMAB Clone 18 (DSM ACC 2587)) as second antigen-binding site binding to IGF-1R.

The relevant full (modified) heavy chains amino acid sequences of the bispecific antibody molecules of N-scFabSS-salt-bridge-s3 are SEQ ID NO: 40-41 and N-scFabSS-salt-bridge-w3C are SEQ ID NO: 42-43

The expression yields, purity, in vitro and in vivo properties of, bispecific, bivalent ScFab-Fc fusion <EGFR-IGF1R> antibody molecules N-scFabSS-salt-bridge-s3 and N-scFabSS-salt-bridge-w3C are determined according to the examples described above.

### Example 22

### Expression & Purification, In Vitro and In Vivo Properties of Bispecific, Trivalent ScFab-IgG Fusion <EGFR-IGF1R> Antibody Molecules KiH-C-scFab-1 and KiH-C-scFab-2

Analogous to the procedures described in Examples 1 and 9, 17, bispecific, trivalent ScFab-IgG fusion <EGFR-IGF1R> antibody molecules KiH-C-scFab-1 and KiH-C-scFab-2 (fusion of a scFab specific for IGF1R to the C-terminus of only one heavy chain of a full length EGFR specific antibody (or vice versa) under usage of the knobs-into-holes technology) are expressed and purified. These bispecific <EGFR-IGF-1R> antibodies are also based on the heavy chain variable domains of SEQ ID NO: 8, and the light chain variable domains of SEQ ID NO: 10 (derived from humanized <EGFR>ICR62) as first antigen-binding site binding to EGFR, and on the heavy chain variable domains of SEQ ID NO: 23, and the light chain variable domains of SEQ ID NO: 25 (derived from the human anti-IGF-1R antibodies <IGF-1R> HUMAB Clone 18 (DSM ACC 2587)) as second antigen-binding site binding to IGF-1R.

The relevant full (modified) heavy and light chains amino acid sequences of the bispecific antibody molecules N-scFabSS are SEQ ID NO: 44-46, and of N-scFabSS-salt-bridge-s3 are SEQ ID NO: 47-49.

The expression yields, purity, in vitro and in vivo properties of, bispecific, bivalent ScFab-Fc fusion <EGFR-IGF1R> antibody molecules N-scFabSS, N-scFabSS-salt-bridge-s3 and N-scFabSS-salt-bridge-w3C are determined according to the examples described above.

1. A bispecific antibody binding to EGFR and IGF-1R comprising a first antigen-binding site that binds to EGFR and a second antigen-binding site that binds to IGF-1R, characterized in that
i) said antigen-binding sites are each a pair of an antibody heavy chain variable domain and an antibody light chain variable domain;
ii) said first antigen-binding site comprises in the heavy chain variable domain a CDR3 region of SEQ ID NO: 1, a CDR2 region of SEQ ID NO: 2, and a CDR1 region of SEQ ID NO:3, and in the light chain variable domain a CDR3 region of SEQ ID NO: 4, a CDR2 region of SEQ ID NO:5, and a CDR1 region of SEQ ID NO:6; and
iii) said second antigen-binding site comprises in the heavy chain variable domain a CDR3 region of SEQ ID NO: 11, a CDR2 region of SEQ ID NO: 12, and a CDR1 region of SEQ ID NO:13, and in the light chain variable domain a CDR3 region of SEQ ID NO: 14, a CDR2 region of SEQ ID NO:15, and a CDR1 region of SEQ ID NO:16;
or said second antigen-binding site comprises in the heavy chain variable domain a CDR3 region of SEQ ID NO: 17, a CDR2 region of SEQ ID NO: 18, and a CDR1 region of SEQ ID NO:19, and in the light chain variable domain a CDR3 region of SEQ ID NO: 20, a CDR2 region of SEQ ID NO:21, and a CDR1 region of SEQ ID NO:22.

2. The bispecific antibody according to claim 1, characterized in that
said second antigen-binding site comprises in the heavy chain variable domain a CDR3 region of SEQ ID NO: 11, a CDR2 region of SEQ ID NO: 12, and a CDR1 region of SEQ ID NO:13, and in the light chain variable domain a CDR3 region of SEQ ID NO: 14, a CDR2 region of SEQ ID NO:15, and a CDR1 region of SEQ ID NO:16.

3. The bispecific antibody according to claim 1, characterized in that
said second antigen-binding site comprises in the heavy chain variable domain a CDR3 region of SEQ ID NO: 17, a CDR2 region of SEQ ID NO: 18, and a CDR1 region of SEQ ID NO:19, and in the light chain variable domain a CDR3 region of SEQ ID NO: 20, a CDR2 region of SEQ ID NO:21, and a CDR1 region of SEQ ID NO:22.

4. The bispecific antibody according to claim 1, characterized in that
i) said first antigen-binding site comprises as heavy chain variable domain SEQ ID NO: 7 or SEQ ID NO: 8, and as light chain variable domain SEQ ID NO: 9 or SEQ ID NO: 10
ii) said second antigen-binding site comprises as heavy chain variable domain SEQ ID NO: 23 or SEQ ID NO: 24, and as light chain variable domain a SEQ ID NO: 25 or SEQ ID NO: 26.

5. The bispecific antibody according to claim 1, characterized in that
i) said first antigen-binding site comprises as heavy chain variable domain SEQ ID NO: 8, and as light chain variable domain SEQ ID NO: 10,
ii) said second antigen-binding site comprises as heavy chain variable domain SEQ ID NO: 23, and as light chain variable domain a SEQ ID NO: 25.

6. The bispecific antibody according to claim 1, characterized in that said antibody is bivalent, trivalent or tetravalent.

7. The bispecific antibody according to claim 1, characterized in that said antibody is glycosylated with a sugar chain at Asn297 whereby the amount of fucose within said sugar chain is 65% or lower.

8. A pharmaceutical composition comprising a bispecific antibody according to claim 1.
